SEQUENCE LISTING (59 sequences)

SEQ ID NO: 1 - protein, 214 aa, synthetic construct
  REGION 1..214: note = Synthetic peptide
AQVLTQTPSPVSAAVGGTVTISCQSSQSVYNNNNLVWYQQKPGQPPKQVIYKASKVASGVPSRFKGSGSGTQFTLTISDLECDDAATYYCLGGYSGDIYTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 2 - protein, 8 aa, synthetic construct
  REGION 1..8: note = Synthetic peptide
QSVYNNNN

SEQ ID NO: 3 - protein, 7 aa, synthetic construct
  REGION 1..7: note = Synthetic peptide
KASKVAS

SEQ ID NO: 4 - protein, 10 aa, synthetic construct
  REGION 1..10: note = Synthetic peptide
LGGYSGDIYT

SEQ ID NO: 5 - protein, 439 aa, synthetic construct
  REGION 1..439: note = Synthetic peptide
CQSVEESGGRLVTPGTPLTLTCTASGFTISSYHMSWVRQAPGKGLEWIGYISTSGNIYYASWAKGRFTISKTSSTTVDLRMTSLTTEDTATYFCARLGIATGYSFWGHGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 6 - protein, 10 aa, synthetic construct
  REGION 1..10: note = Synthetic peptide
GFTISSYHMS

SEQ ID NO: 7 - protein, 7 aa, synthetic construct
  REGION 1..7: note = Synthetic peptide
ISTSGNI

SEQ ID NO: 8 - protein, 11 aa, synthetic construct
  REGION 1..11: note = Synthetic peptide
ARLGIATGYSF

SEQ ID NO: 9 - protein, 214 aa, synthetic construct
  REGION 1..214: note = Synthetic peptide
AQVLTQTPSPVSAAVGGTVTISCQSSQSVYNNNNLVWFQKKPGQPPKQLIYKASKVASGVPSRFKGSGSGTQFTLTISDLECDDAATYYCLGGYSGDIYTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 10 - protein, 8 aa, synthetic construct
  REGION 1..8: note = Synthetic peptide
QSVYNNNN

SEQ ID NO: 11 - protein, 7 aa, synthetic construct
  REGION 1..7: note = Synthetic peptide
KASKVAS

SEQ ID NO: 12 - protein, 10 aa, synthetic construct
  REGION 1..10: note = Synthetic peptide
LGGYSGDIYT

SEQ ID NO: 13 - protein, 439 aa, synthetic construct
  REGION 1..439: note = Synthetic peptide
QEQLKESGGGLVTPGGTLTLTCTASGFTISSYHMSWVRQAPGKGLEWIGYISTSGNIYYATWAKGRFTISKTSSTTVDLRMTSLTTEDTATYFCARLGIATGYSFWGHGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 14 - protein, 10 aa, synthetic construct
  REGION 1..10: note = Synthetic peptide
GFTISSYHMS

SEQ ID NO: 15 - protein, 7 aa, synthetic construct
  REGION 1..7: note = Synthetic peptide
ISTSGNI

SEQ ID NO: 16 - protein, 11 aa, synthetic construct
  REGION 1..11: note = Synthetic peptide
ARLGIATGYSF

SEQ ID NO: 17 - protein, 220 aa, synthetic construct
  REGION 1..220: note = Synthetic peptide
DIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 17 aa, synthetic construct
  REGION 1..17: note = Synthetic peptide
KSIQTLLYSSNQKNYLA

SEQ ID NO: 19 - protein, 7 aa, synthetic construct
  REGION 1..7: note = Synthetic peptide
WASIRKS

SEQ ID NO: 20 - protein, 9 aa, synthetic construct
  REGION 1..9: note = Synthetic peptide
QQYYSYPLT

SEQ ID NO: 21 - protein, 446 aa, synthetic construct
  REGION 1..446: note = Synthetic peptide
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 5 aa, synthetic construct
  REGION 1..5: note = Synthetic peptide
NYGMS

SEQ ID NO: 23 - protein, 17 aa, synthetic construct
  REGION 1..17: note = Synthetic peptide
SISSGGGSTYYPDNVKG

SEQ ID NO: 24 - protein, 7 aa, synthetic construct
  REGION 1..7: note = Synthetic peptide
GGAGIDY

SEQ ID NO: 25 - protein, 113 aa, synthetic construct
  REGION 1..113: note = Synthetic peptide
DIQMTQSPSSLSASVGDRVTITCKSIQTLLYSSNQKNYLAWYQQKPGKAPKLLIYWASIRKSGVPSRFSGSGSGTDFTLTISSLQPEDFATYYCQQYYSYPLTFGGGTKLEIK

SEQ ID NO: 26 - protein, 17 aa, synthetic construct
  REGION 1..17: note = Synthetic peptide
KSIQTLLYSSNQKNYLA

SEQ ID NO: 27 - protein, 7 aa, synthetic construct
  REGION 1..7: note = Synthetic peptide
WASIRKS

SEQ ID NO: 28 - protein, 9 aa, synthetic construct
  REGION 1..9: note = Synthetic peptide
QQYYSYPLT

SEQ ID NO: 29 - protein, 116 aa, synthetic construct
  REGION 1..116: note = Synthetic peptide
EVQLVESGGGLVQPGGSLRLSCAASGFTFSNYGMSWVRQAPGKGLEWVASISSGGGSTYYPDNVKGRFTISRDNAKNSLYLQMNSLRAEDTAVYYCARGGAGIDYWGQGTLVTVSS

SEQ ID NO: 30 - protein, 5 aa, synthetic construct
  REGION 1..5: note = Synthetic peptide
NYGMS

SEQ ID NO: 31 - protein, 17 aa, synthetic construct
  REGION 1..17: note = Synthetic peptide
SISSGGGSTYYPDNVKG

SEQ ID NO: 32 - protein, 7 aa, synthetic construct
  REGION 1..7: note = Synthetic peptide
GGAGIDY

SEQ ID NO: 33 - protein, 113 aa, synthetic construct
  REGION 1..113: note = Synthetic peptide
DVVMTQSPDSLAVSLGERATINCKSSQNVLYSSNNKNYLAWYQQKPGHPPKLLIYWASTRESGVPDRFSGSGSGTDFTLTITSLQTEDVAVYYCQQYYSSPLTFGGGTKVEIK

SEQ ID NO: 34 - protein, 17 aa, synthetic construct
  REGION 1..17: note = Synthetic peptide
KSSQNVLYSSNNKNYLA

SEQ ID NO: 35 - protein, 7 aa, synthetic construct
  REGION 1..7: note = Synthetic peptide
WASTRES

SEQ ID NO: 36 - protein, 9 aa, synthetic construct
  REGION 1..9: note = Synthetic peptide
QQYYSSPLT

SEQ ID NO: 37 - protein, 124 aa, synthetic construct
  REGION 1..124: note = Synthetic peptide
EVQLVESGAEVKKPGASVKVSCKASGYTFTNYAMHWVRQAPGQRLEWMGWINAGNGKRKYSQKFQDRVTINRDTSASTIYMELSSLGSEDTAVYYCAREEDHAGSGSYLSMDVWGQGTLVTVSS

SEQ ID NO: 38 - protein, 5 aa, synthetic construct
  REGION 1..5: note = Synthetic peptide
NYAMH

SEQ ID NO: 39 - protein, 17 aa, synthetic construct
  REGION 1..17: note = Synthetic peptide
WINAGNGKRKYSQKFQD

SEQ ID NO: 40 - protein, 15 aa, synthetic construct
  REGION 1..15: note = Synthetic peptide
EEDHAGSGSYLSMDV

SEQ ID NO: 41 - protein, 108 aa, synthetic construct
  REGION 1..108: note = Synthetic peptide
QSVLTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVL

SEQ ID NO: 42 - protein, 11 aa, synthetic construct
  REGION 1..11: note = Synthetic peptide
SGEALPMQFAH

SEQ ID NO: 43 - protein, 7 aa, synthetic construct
  REGION 1..7: note = Synthetic peptide
KDSERPS

SEQ ID NO: 44 - protein, 11 aa, synthetic construct
  REGION 1..11: note = Synthetic peptide
QSPDSTNTYEV

SEQ ID NO: 45 - protein, 113 aa, synthetic construct
  REGION 1..113: note = Synthetic peptide
EVQLVQSGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSS

SEQ ID NO: 46 - protein, 5 aa, synthetic construct
  REGION 1..5: note = Synthetic peptide
KAWMS

SEQ ID NO: 47 - protein, 19 aa, synthetic construct
  REGION 1..19: note = Synthetic peptide
RIKSTADGGTTSYAAPVEG

SEQ ID NO: 48 - protein, 4 aa, synthetic construct
  REGION 1..4: note = Synthetic peptide
TSAH

SEQ ID NO: 49 - protein, 113 aa, synthetic construct; identical to SEQ ID NO: 45
  REGION 1..113: note = Synthetic peptide

SEQ ID NO: 50 - protein, 108 aa, synthetic construct; identical to SEQ ID NO: 41
  REGION 1..108: note = Synthetic peptide

SEQ ID NO: 51 - protein, 5 aa, synthetic construct
  REGION 1..5: note = Synthetic peptide
NYAMH

SEQ ID NO: 52 - protein, 17 aa, synthetic construct
  REGION 1..17: note = Synthetic peptide
WINAGNGKRKYSQKFQD

SEQ ID NO: 53 - protein, 15 aa, synthetic construct
  REGION 1..15: note = Synthetic peptide
EEDHAGSGSYLSMDV

SEQ ID NO: 54 - protein, 17 aa, synthetic construct
  REGION 1..17: note = Synthetic peptide
KSSQNVLYSSNNKNYLA

SEQ ID NO: 55 - protein, 7 aa, synthetic construct
  REGION 1..7: note = Synthetic peptide
WASTRES

SEQ ID NO: 56 - protein, 9 aa, synthetic construct
  REGION 1..9: note = Synthetic peptide
QQYYSSPLT

SEQ ID NO: 57 - protein, 116 aa, synthetic construct
  REGION 1..116: note = Synthetic peptide
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSS

SEQ ID NO: 58 - protein, 113 aa, synthetic construct
  REGION 1..113: note = Synthetic peptide
DIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEIK

SEQ ID NO: 59 - protein, 14 aa, synthetic construct
  REGION 1..14: note = corresponds to residues 122-135 of human alpha-synuclein
  SITE 8: note = MISC_FEATURE - serine residue is phosphorylated
NEAYEMPSEEGYQD

(19) United States
(12) Patent Application Publication
(10) Pub. No.: US 2026/0029411 A1
(43) Pub. Date: Jan. 29, 2026

(54) DETERMINATION OF PARKINSON'S DISEASE

(71) Applicants: Hoffmann-La Roche Inc., Little Falls, NJ (US); Roche Diagnostic Operations, Inc., Indianapolis, IN (US); Prothena Biosciences Limited, South San Francisco, CA (US); Ventana Medical Systems, Inc., Tucson, AZ (US)

(72) Inventors: Christian Czech, Basel (CH); Sebastian Dziadek, Basel (CH); Lidija Pestic-Dragovich, Tucson, AZ (US); Lei Tang, Tucson, AZ (US); Thomas Kremer, Basel (CH); Wagner Marcelo Zago, Dublin (IE); Tsu-Shuen Tsao, Tucson, AZ (US); Adriana Racolta, Tucson, AZ (US); Marta Canamero, Penzberg (DE); Ronald Torres, Dublin (IE); Mirko Ritter, Penzberg (DE)

(21) Appl. No.: 19/257,106

(22) Filed: Jul. 1, 2025

Related U.S. Application Data
(62) Division of application No. 17/265,154, now Pat. No. 12,372,537.
(60) Provisional application No. 62/716,504, filed on Aug. 9, 2018.

(51) Int. Cl. G01N 33/68

(52) U.S. Cl. CPC G01N 33/6893; G01N 2800/2835

(57) ABSTRACT

The invention provides methods and compositions for accurate identification and determination of Parkinson's disease ante-mortem tissue samples. The determination of Parkinson's disease is based on the binding of localized phosphorylated alpha-synuclein with the nerve feature. The methods disclosed in the invention may be used on myriad tissue types and could be manual or automated.

### CROSS REFERENCE TO RELATED APPLICATIONS

The present application is a divisional of U.S. patent application Ser. No. 17/265,154, filed Feb. 1, 2021, which is a U.S. National Phase Application pursuant to 35 U.S.C. § 371 of International Application No. PCT/US2019/045793 filed Aug. 8, 2019, which claims priority to U.S. Provisional Patent Application No. 62/716,504 filed Aug. 9, 2018. The entire disclosure contents of these applications are herewith incorporated by reference into the present application.

### INCORPORATION BY REFERENCE OF SEQUENCE LISTING PROVIDED ELECTRONICALLY

This application contains a Sequence Listing submitted as an electronic text file named “17-1472-WO-US-CON Sequence Listing_ST26.xml” having a size in bytes of 76 kb, and created on Jun. 27, 2025. The information contained in this electronic file is hereby incorporated by reference in its entirety.

### BACKGROUND

### Field

The disclosure is directed to methods and compositions for accurate identification of Parkinson's disease. More particularly, the disclosure is directed to the determination of Parkinson's disease in ante-mortem tissue samples.

### Description of Related Art

Parkinson's disease (PD) is currently evaluated by clinical examination of a subject's symptoms and imaging of dopamine transporter function in the brain (DaTscan). Development of therapeutic agents for Parkinson's disease is hampered by a lack of diagnostic tests that accurately identify subjects with PD. The definitive diagnosis of PD is the presence of aggregated alpha-synuclein (aSyn) in neurons combined with loss of dopaminergic neurons in substantia nigra region of the brain, and can only take place post-mortem.

### SUMMARY

The inventors have identified a need in the art to definitively and accurately identify Parkinson's Disease in a living subject.

Provided herein are methods for determining whether a subject has Parkinson's Disease (PD), the method comprising: (a) contacting a biological sample comprising at least one nerve feature from the subject with a primary antibody capable of binding phosphorylated alpha-synuclein; (b) detecting whether the primary antibody capable of binding phosphorylated alpha-synuclein localizes within the nerve feature of the biological sample; and (c) determining the subject has PD when the primary antibody capable of binding phosphorylated alpha-synuclein localizes within the nerve feature.

In some of the methods, the sample comprises a tissue sample. In some of the methods, the nerve feature comprises a nerve cell. In some of the methods, the nerve feature comprises a former nerve cell. In some of the methods, the nerve feature is adjacent to a nerve cell.

In some of the methods, the sample is contacted with at least one protease before being contacted with the primary antibody capable of binding phosphorylated alpha-synuclein. In some embodiments, the method further comprises contacting the sample with at least one phosphatase.

In some of the methods, the method further comprises contacting the biological sample from the subject with a primary antibody capable of binding the nerve feature. In some embodiments, the primary antibody capable of binding to the nerve feature is selected from an antibody capable of binding a protein selected from the group consisting of: ubiquitin C-terminal hydrolase L1 (PGP9.5, UCHL1, NDGOA; PARK5; PGP95; SPG79; Uch-L1; HEL-117; PGP 9.5; HEL-S-53), RNA binding fox-1 homolog 3 (RBFOX3, FOX3, NEUN, FOX-3, HRNBP3), microtubule associated protein 2 (MAP2, MAP2A, MAP2B, MAP2C), 160 kDa neurofilament medium (NEFM, NFM, NEF3, NF-M), 200 kDa neurofilament heavy (NEFH, NFH, CMT2CC), synaptophysin (SYP, MRX96, MRXSYP), and discs large MAGUK scaffold protein 4 (DLG4, DLGH4, PSD-95, PSD95, SAP-90, SAP90, SAP90A).

In some of the methods, the detecting comprising histochemical analysis. In some of the methods, the primary antibody capable of binding phosphorylated alpha-synuclein detects alpha-synuclein phosphorylated at residue S129.

In some of the methods, the sample is fixed. In some embodiments, the sample is a formalin fixed, paraffin embedded (FFPE) sample. In some of the methods, the sample is a frozen sample. In some of the methods, the sample comprises a section of the nerve feature. In some of the methods, the sample is selected from the group consisting of skin tissue, colon tissue, and submandibular gland.

In some of the methods, the primary antibody capable of binding phosphorylated alpha-synuclein and the primary antibody capable of binding to the nerve feature are from the same host species, wherein the host species is a mouse or a rabbit.

In some of the methods, step (a) further comprises contacting the sample with a first secondary antibody having a first label conjugated thereto, wherein the first secondary antibody is immunoreactive with the primary antibody capable of binding phosphorylated alpha-synuclein. In some embodiments, the method comprises contacting the sample with a set of reagents reactive with the first label of the first secondary antibody to generate a first detectable signal in proximity to phosphorylated alpha-synuclein in the sample.

In some of the methods, before contacting the sample with the primary antibody capable of binding to the nerve feature, the method comprises denaturing the immunocomplexes in the sample by incubating the sample at 100° C. for at least 15 minutes. In some embodiments, the method further comprises contacting the sample with a second secondary antibody having a second label conjugated thereto, wherein the second secondary antibody is immunoreactive with the primary antibody capable of binding the nerve feature. In some embodiments, the method comprises contacting the sample with a set of reagents reactive with the second label of the second secondary antibody to generate a second detectable signal in proximity to the nerve feature in the sample. In some embodiments, the first detectable signal and the second detectable signal are different. In some embodiments, the first detectable signal is silver stain and the second detectable signal is QM-Dabsyl or Fast-Red.

In some of the methods, the subject is suspected of having PD.

Also provided herein are kits comprising: (a) a primary antibody capable of binding phosphorylated alpha-synuclein; and (b) a primary antibody capable of binding to a nerve feature. In some embodiments, the kit further comprises: (c) a first secondary antibody having a first label conjugated thereto, wherein the first secondary antibody is immunoreactive with the primary antibody capable of binding phosphorylated alpha-synuclein; (d) a set of reagents that generates a first detectable single when reacted with the first label of the first secondary antibody; (e) a second secondary antibody having a second label conjugated thereto, wherein the second secondary antibody is immunoreactive with the primary antibody capable of binding the nerve feature; and (f) a set of reagents that generates a second detectable single when reacted with the second label of the second secondary antibody; wherein the first detectable signal and the second detectable signal are different. In some embodiments, the first detectable signal is silver stain and the second detectable signal is QM-Dabsyl or Fast-Red.

In some of the kits, the primary antibody capable of binding to the nerve feature is selected from an antibody capable of binding a protein selected from the group consisting of: ubiquitin C-terminal hydrolase L1 (PGP9.5, UCHL1, NDGOA; PARK5; PGP95; SPG79; Uch-L1; HEL-117; PGP 9.5; HEL-S-53), RNA binding fox-1 homolog 3 (RBFOX3, FOX3, NEUN, FOX-3, HRNBP3), microtubule associated protein 2 (MAP2, MAP2A, MAP2B, MAP2C), 160 kDa neurofilament medium (NEFM, NFM, NEF3, NF-M), 200 kDa neurofilament heavy (NEFH, NFH, CMT2CC), synaptophysin (SYP, MRX96, MRXSYP), and discs large MAGUK scaffold protein 4 (DLG4, DLGH4, PSD-95, PSD95, SAP-90, SAP90, SAP90A).

Also provided herein are methods for determining whether a subject has Parkinson's Disease (PD), the method comprising: (a) preparing a fixed or frozen section of a biological sample from a subject suspected of having PD; (b) detecting whether phosphorylated alpha-synuclein localizes within a nerve feature in the section; and (c) diagnosing the subject with PD when phosphorylated alpha-synuclein localizes within a nerve feature. In some embodiments, step (c) further comprises: (d) contacting the section with a primary antibody capable of binding phosphorylated alpha-synuclein and a primary antibody capable of binding the nerve feature.

In some of the methods, the primary antibody capable of binding to the nerve feature is selected from an antibody capable of binding a protein selected from the group consisting of: ubiquitin C-terminal hydrolase L1 (PGP9.5, UCHL1, NDGOA; PARK5; PGP95; SPG79; Uch-L1; HEL-117; PGP 9.5; HEL-S-53), RNA binding fox-1 homolog 3 (RBFOX3, FOX3, NEUN, FOX-3, HRNBP3), microtubule associated protein 2 (MAP2, MAP2A, MAP2B, MAP2C), 160 kDa neurofilament medium (NEFM, NFM, NEF3, NF-M), 200 kDa neurofilament heavy (NEFH, NFH, CMT2CC), synaptophysin (SYP, MRX96, MRXSYP), and discs large MAGUK scaffold protein 4 (DLG4, DLGH4, PSD-95, PSD95, SAP-90, SAP90, SAP90A).

In some of the methods, the method further comprises: contacting the section with a first secondary antibody having a first label conjugated thereto, wherein the first secondary antibody is immunoreactive with the primary antibody capable of binding phosphorylated alpha-synuclein, and contacting the section with a second secondary antibody having a second label conjugated thereto, wherein the second secondary antibody is immunoreactive with the primary antibody capable of binding the nerve feature. In some embodiments, the method further comprises: contacting the section with a set of reagents reactive with the first label of the first secondary antibody to generate a first detectable signal in proximity to phosphorylated alpha-synuclein in the sample; and contacting the section with a set of reagents reactive with the second label of the second secondary antibody to generate a second detectable signal in proximity to the nerve feature in the sample. In some embodiments, the method further comprises denaturing immunocomplexes in the sample after contacting the section with the primary antibody capable of binding phosphorylated alpha-synuclein and before contacting the section with the primary antibody capable of binding the nerve feature.

In some of the methods, the section is contacted with at least one protease before being contacted with a primary antibody. In some embodiments, the method further comprises contacting the section with at least one phosphatase.

In some of the methods, the primary antibody capable of binding phosphorylated alpha-synuclein and the primary antibody capable of binding the nerve feature are from the same host species.

In some of the methods, the first detectable signal and the second detectable signal are different. In some embodiments, the first detectable signal is silver stain and the second detectable signal is QM-Dabsyl or Fast-Red.

Also provided herein are methods of diagnosing PD in a subject, said method comprising: (a) obtaining a biological sample comprising at least one nerve feature from a subject suspected of having PD; (b) detecting whether phosphorylated alpha-synuclein localizes with the nerve feature in the sample by contacting the sample with an anti-PGP.5 antibody and determining co-localization between phosphorylated alpha-synuclein and PGP9.5; and (c) diagnosing the subject with PD when it is determined in the affirmative that there is co-localization between phosphorylated alpha-synuclein and PGP9.5 in the nerve feature.

Also provided herein are methods of diagnosing and treating PD in a subject, said method comprising: (a) obtaining a biological sample comprising at least one nerve feature from a subject suspected of having PD; (b) detecting whether phosphorylated alpha-synuclein localizes with the nerve feature in the sample by contacting the sample with an anti-PGP.5 antibody and determining co-localization between phosphorylated alpha-synuclein and PGP9.5; (c) diagnosing the subject with PD when it is determined in the affirmative that there is co-localization between phosphorylated alpha-synuclein and PGP9.5 in the nerve feature; and (d) administering a therapy to treat PD in the subject diagnosed as having PD.

Also provided herein are methods of treating a subject diagnosed with PD, comprising administering to the subject an effective regime of an alpha-synuclein antibody, wherein an antibody capable of binding phosphorylated alpha-synuclein and an antibody capable of binding a nerve feature have been shown to co-localize in a nerve feature in a skin sample from the subject.

Also provided herein are methods of treating a subject determined to have PD, comprising administering to the subject an effective regime of an alpha-synuclein antibody, wherein the subject was determined to have PD by any one of the methods disclosed herein.

In some such methods, the effective regime of an alpha-synuclein antibody comprises an alpha-synuclein antibody selected from the group consisting of: a monoclonal antibody binding within residues 1-20 of alpha-synuclein, 1-10 of alpha synuclein, 4-15 of alpha-synuclein, 91-99 of alpha-synuclein, 117-123 of alpha-synuclein, 118-126 of alpha-synuclein, prasinezumab (PRX002), a humanized antibody having the CDR's of antibody clone 1H7 (ATCC Accession No. PTA-8220), a humanized antibody having the CDR's of antibody clone 9E4 (ATCC Accession No. PTA-8221), the CDR's of antibody clone NI-202.21D11, and the CDR's of antibody clone NI-202.12F4, such as, for example, alpha-synuclein antibodies disclosed in U.S. Pat. Nos. 8,092,801, 8,609,820, 8,790,644, 8,940,276, 9,580,493, which are incorporated by reference herein in their entirety. Some such antibodies comprise a VH CDR1 comprising residues 31-35 of SEQ ID NO:49, a VH CDR2 comprising residues 50-68 of SEQ ID NO:49, a VH CDR3 comprising residues 101-102 of SEQ ID NO:49, a VL CDR1 comprising residues 23-33 of SEQ ID NO:50, a VL CDR2 comprising residues 49-55 of SEQ ID NO:50, and a VL CDR3 comprising residues 88-98 of SEQ ID NO:50. Some such antibodies comprise a VH CDR1 comprising SEQ ID NO:51, a VH CDR2 comprising SEQ ID NO:52, a VH CDR3 comprising SEQ ID NO:53, a VL CDR1 comprising SEQ ID NO:54, a VL CDR2 comprising SEQ ID NO:55 and a VL CDR3 comprising SEQ ID NO:56. Some such antibodies comprise a heavy chain comprising SEQ ID NO:57 and a light chain comprising SEQ ID NO:58.

In some such methods, the antibody is prasinezumab (PRX002). In some such methods, the antibody comprises three light CDRs designated SEQ ID NOs: 18-20 respectively and three heavy chain CDRs designated SEQ ID NOs: 22-24 respectively. In some such methods, the antibody comprises a light chain designated SEQ ID NO:17 and a heavy chain designated SEQ ID NO:21

In some such methods, the antibody is 9E4, as disclosed in U.S. Pat. No. 8,609,820, which is incorporated by reference herein in its entirety. In some such methods, the antibody comprises three light CDRs designated SEQ ID NOs:26-28 respectively and three heavy chain CDRs designated SEQ ID NOs:30-32 respectively. In some such methods, the antibody comprises a light chain designated SEQ ID NO:25 and a heavy chain designated SEQ ID NO:29.

In some such methods, the antibody is NI-202.21D11. In some such methods, the antibody comprises three light CDRs designated SEQ ID NOs:34-36 respectively and three heavy chain CDRs designated SEQ ID NOs: 38-40 respectively. In some such methods, the antibody comprises a light chain variable region designated SEQ ID NO:33 and a heavy chain variable region designated SEQ ID NO:37.

In some such methods, the antibody is NI-202.12F4. In some such methods, the antibody comprises light heavy CDRs designated SEQ ID NOs:42-44 respectively and three heavy chain CDRs designated SEQ ID NOs: 46-48 respectively. In some such methods, the antibody comprises a light chain variable region designated SEQ ID NO:41 and a heavy chain variable region designated SEQ ID NO:45.

Also provided herein are methods for detecting phosphorylated alpha-synuclein, the method comprising: contacting a biological sample with a primary antibody capable of binding phosphorylated alpha-synuclein, and detecting the primary antibody capable of binding phosphorylated alpha-synuclein. In some of the methods, the sample is contacted with at least one protease before being contacted with the primary antibody capable of binding phosphorylated alpha-synuclein. In some methods, the method further comprises contacting the sample with at least one phosphatase. In some methods, the detecting comprising histochemical analysis. In some methods, the primary antibody capable of binding phosphorylated alpha-synuclein detects alpha-synuclein phosphorylated at residue S129. In certain methods, the primary antibody capable of binding phosphorylated alpha-synuclein is the 7E2 antibody clone or the 3G2 antibody clone. In some methods, the sample is fixed. In certain methods, the sample is a formalin fixed, paraffin embedded (FFPE) sample. In some methods, the sample is a frozen sample

In some methods, the method further comprises contacting the sample with a first secondary antibody having a first label conjugated thereto, wherein the first secondary antibody is immunoreactive with the primary antibody capable of binding phosphorylated alpha-synuclein. In some methods, the method comprises contacting the sample with a set of reagents reactive with the first label of the first secondary antibody to generate a first detectable signal in proximity to phosphorylated alpha-synuclein in the sample.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A shows exemplary staining of alpha-synuclein phosphorylated at S129 in brain tissue from a subject with PD using the anti-alpha-synuclein antibody and concentration as indicated (MJF-R13 (8-8) top panel; P-syn/81A middle panel; 5H5 bottom panel).

FIG. 1B shows exemplary staining in brain tissue from a subject with PD using the anti-phosphorylated S129 alpha-synuclein antibody and concentration as indicated (2G11 top panel; 7E2 middle panel; 3G2 bottom panel).

FIG. 1C shows exemplary staining in brain tissue from a subject with PD using the anti-phosphorylated S129 alpha-synuclein antibody and concentration as indicated (11A5 top panel; pSyn #64 (WAKO) bottom panel).

FIG. 2A shows no staining was observed with 3G2 and 7E2 phosphorylated S129 alpha-synuclein antibodies without antigen retrieval in brain tissue from non-PD subjects (7E2 middle panel (0.08 g/mL); 3G2 bottom panel (0.08 g/mL)). Staining total alpha-synuclein by antibody 5C12 (0.5 g/ml) is shown in the top panel. No protease or phosphatase treatment. Images were taken at 10× magnification. Antibodies were diluted in DISCOVERY Goat Ig Block.

FIG. 2B shows no staining was observed with 3G2 and 7E2 phosphorylated S129 alpha-synuclein antibodies without antigen retrieval in brain tissue from non-PD subjects (7E2 middle panel (0.08 g/mL); 3G2 bottom panel (0.08 g/mL)). Staining total alpha-synuclein by antibody 5C12 (0.5 g/ml) is shown in the top panel. No protease or phosphatase treatment. Images were taken at 10× magnification. Antibodies were diluted in DISCOVERY Goat Ig Block.

FIG. 3A shows very faint cytoplasmic staining (top panel) in non-PD brain tissue with the anti-phosphorylated S129 alpha-synuclein 7E2 antibody after antigen retrieval (cell conditioning) that is absent after phosphatase treatment (bottom panel). Alkaline phosphatase treatment was 50 g/mL for 2 hours.

FIG. 3B shows very faint cytoplasmic staining (top panel) in non-PD brain tissue with the anti-phosphorylated S129 alpha-synuclein 3G2 antibody after antigen retrieval (cell conditioning) that is absent after phosphatase treatment (bottom panel). Alkaline phosphatase treatment was 50 g/mL for 2 hours.

FIG. 4A shows that Lewy body staining by the MJF-R13(8-8) antibody in cortical brain tissue from a subject with PD was severely decreased by alkaline phosphatase treatment (lower panel; 50 g/mL for 2 hours). Images were taken at 10× magnification. MJF-R13(8-8) antibody (0.4 g/mL) was diluted in DISCOVERY Goat Ig Block.

FIG. 4B shows that Lewy body staining by the 81A antibody in cortical brain tissue from a subject with PD was severely decreased by alkaline phosphatase treatment (lower panel; 50 g/mL for 2 hours). Images were taken at 10× magnification. 81A antibody (2.0 g/mL) was diluted in DISCOVERY Goat Ig Block.

FIG. 4C shows that Lewy body staining by the 11A5 antibody in cortical brain tissue from a subject with PD was moderately decreased by alkaline phosphatase treatment (lower panel; 50 g/mL for 2 hours). Images were taken at 10× magnification. 11A5 antibody (0.4 g/mL) was diluted in DISCOVERY Goat Ig Block.

FIG. 4D shows that Lewy body staining by the 7E2 antibody in cortical brain tissue from a subject with PD was moderately decreased by alkaline phosphatase treatment (lower panel; 50 g/mL for 2 hours). Images were taken at 10× magnification. 7E2 antibody (0.08 g/mL) was diluted in DISCOVERY Goat Ig Block.

FIG. 4E shows that Lewy body staining by the 3G2 antibody in cortical brain tissue from a subject with PD was moderately decreased by alkaline phosphatase treatment (lower panel; 50 g/mL for 2 hours). Images were taken at 10× magnification. 3G2 antibody (0.08 g/mL) was diluted in DISCOVERY Goat Ig Block.

FIG. 5A shows that protease treatment (VENTANA® Protease 1 at 36° C. for 4 minutes) reduced non-Lewy body staining by the 11A5 antibody in cortical brain tissue from a subject with PD without decreasing Lewy body staining. Images were taken at 10× magnification. 11A5 antibody (0.4 g/mL) was diluted in DISCOVERY Goat Ig Block.

FIG. 5B shows that protease treatment (VENTANA® Protease 1 at 36° C. for 4 minutes) reduced non-Lewy body staining by the 7E2 antibody in cortical brain tissue from a subject with PD without decreasing Lewy body staining. Images were taken at 10× magnification. 7E2 antibody (0.08 g/mL) was diluted in DISCOVERY Goat Ig Block.

FIG. 5C shows that protease treatment (VENTANA® Protease 1 at 36° C. for 4 minutes) reduced non-Lewy body staining by the 3G2 antibody in cortical brain tissue from a subject with PD without decreasing Lewy body staining. Images were taken at 10× magnification. 3G2 antibody (0.08 g/mL) was diluted in DISCOVERY Goat Ig Block.

FIG. 6 shows that phosphorylation specific alpha-synuclein antibodies 7E2 (middle panel) and 3G2 (bottom panel), in absence of protease treatment, produced staining patterns in brain tissue from a subject with PD that were identical to total alpha-synuclein antibody 5C12 (top panel) after protease treatment. Images were taken at 10× magnification. All antibodies were diluted in DISCOVERY Goat Ig Block (5C12 antibody at 0.5 g/mL; 7E2 antibody at 0.08 g/mL; and 3G2 antibody at 0.08 g/mL).

FIG. 7A shows that in absence of protease-based antigen retrieval, the anti-phosphorylated S129 alpha-synuclein 7E2 antibody (bottom panel) detected aggregated alpha-synuclein in skin sections from a subject with PD similar to that detected using the anti-total alpha-synuclein 5C12 antibody (top panel) coupled to VENTANA® Protease 1 treatment (36° C. for 4 minutes).

FIG. 7B shows that in absence of protease-based antigen retrieval, the anti-phosphorylated S129 alpha-synuclein 11A5 antibody (top panel) and 3G2 antibody detected aggregated alpha-synuclein in skin sections from a subject with PD similar to that detected using the anti-total alpha-synuclein 5C12 antibody (FIG. 7A top panel) coupled to VENTANA® Protease 1 treatment (36° C. for 4 minutes).

FIG. 7C shows that in absence of protease-based antigen retrieval, the anti-phosphorylatedS129 alpha-synuclein MJF-R13 antibody (top panel) and P-Syn/81A antibody detected aggregated alpha-synuclein in skin sections from a subject with PD similar to that detected using the anti-total alpha-synuclein 5C12 antibody (FIG. 7A top panel) coupled to VENTANA® Protease 1 treatment (36° C. for 4 minutes).

FIG. 8A shows that DISCOVERY Goat Ig Block results in minimal background while preserving staining intensity of the anti-phosphorylated S129 alpha-synuclein 7E2 antibody (1.0 g/mL) in a PD sample. Images were taken at 10× magnification in top panel and 40× magnification in bottom panel. The images in the top and the bottom panels came from different fields of views of the same tissue section.

FIG. 8B shows that the 90103 diluent results in minimal background while preserving staining intensity of the anti-phosphorylated S129 alpha-synuclein 7E2 antibody (1.0 g/mL) in a PD sample. Images were taken at 10× magnification in top panel and 40× magnification in bottom panel. The images in the top and the bottom panels came from different fields of views of the same tissue section.

FIG. 9 shows the results of testing the anti-phosphorylated S129 alpha-synuclein 7E2 antibody above and below the nominal concentration of 1.0 g/mL (guardbanding experiments) in which quantitative measurements in the form of percent neuronal features stained per slide were performed. For this experiment the tissue sections were not treated with protease nor phosphatase prior to antibody application and detection with chromogen. Staining of skin tissue from a subject with PD with the anti-phosphorylated S129 alpha-synuclein 7E2 antibody at 0.5 g/mL, 0.75 g/mL, 1.0 g/mL, 1.25 g/mL, and 1.5 g/mL diluted in DISCOVERY Goat Ig Block indicates robust staining with antibody concentrations varying +/−50% around the nominal value of 1.0 g/mL.

FIG. 10A shows that the anti-PGP9.5 antibody EPR4118 (0.2 g/mL) stains neuronal features in skin samples from a normal subject (non-PD subject).

FIG. 10B shows that the anti-PGP9.5 antibody 13C4/I3C4 (0.2 g/mL) stains neuronal features in skin samples from a normal subject (non-PD subject).

FIG. 10C shows that the polyclonal anti-PGP9.5 antibody by Cell Marque (1.0 g/mL; VENTANA® catalog number of 760-4434) stains neuronal features in skin samples from a normal subject (non-PD subject).

FIG. 11A shows strong background staining of collagen fibers in freshly cut tissue slides (˜24-48 hours) from a non-PD subject. Staining was performed using the anti-phosphorylated S129 alpha-synuclein 7E2 antibody at 0.1 g/mL and VENTANA OptiView DAB IHC Detection Kit. Tissue section did not undergo CC1 antigen retrieval treatment. Similar results were obtained when the anti-phosphorylated S129 alpha-synuclein 7E2 antibody and OptiView HQ Linker were not applied (data not shown), demonstrating the collagen background staining was produced by OptiView Multimer.

FIG. 11B shows minimal background staining of collagen fibers in tissue slides from a non-PD subject stained approximately 5 months after the slide was cut.

FIG. 12A shows that background staining of collagen fibers in tissue slides can be exacerbated by antigen retrieval with a protease in tissue slides from a non-PD subject. Tissue was tested with the anti-phosphorylated S129 alpha-synuclein 7E2 antibody (0.1 g/mL) diluted in DISCOVERY Goat Ig Block.

FIG. 12B shows that background staining of collagen fibers in tissue slides can be lessened by CC1 antigen retrieval in tissue slides from a non-PD subject. Tissue was incubated with the anti-phosphorylated S129 alpha-synuclein 7E2 antibody (1.0 g/mL) diluted in DISCOVERY Goat Ig Block without prior antigen retrieval with CC1 (top panel) or after antigen retrieval using CC1 for 48 minutes at 100° C.; bottom panel) on VENTANA BenchMark ULTRA automated staining instrument.

FIG. 13 shows that the ultraView Universal DAB Detection Kit (right panels) results in lower staining intensity for PGP9.5 compared with OptiView DAB IHC Detection Kit (left panels) in both PD skin (top panels) and control skin (bottom panels) samples. OptiView detection: HQ Linker+HRP Multimer. ultraView detection: anti-Rb/Ms HRP Multimer. Tissue samples were pretreated with CC1 at 100° C. for 32 minutes, and the PGP9.5 EPR4118 antibody (0.5 g/mL) was diluted in DISCOVERY Goat Ig Block. Images were taken at 40× magnification.

FIG. 14 shows that the amplification process with the ultraView Universal DAB Detection Kit results in a rise in non-specific background staining in both PD skin (top panel) and control skin (bottom panel) samples. Amplification: Ms anti-Rb followed by Rb anti-Ms IgG. ultraView detection: anti-Rb/Ms HRP Multimer. Anti-phosphorylated S129 alpha-synuclein 7E2 antibody (1.0 g/mL) was diluted in DISCOVERY Goat Ig Block. No pre-treatment with CC1 nor protease. Images were taken at 40× magnification.

FIG. 15 shows that the ultraView SISH DNP Detection Kit (top panel) and DISCOVERY Purple kit (middle and bottom panels) both detect aggregated anti-phosphorylated S129 alpha-synuclein in skin samples from a subject with PD with little to no background when used with goat anti-rabbit-RP conjugate stain deposition. Phosphorylated S129 alpha-synuclein stained black (silver stain) or purple (TAMRA). Anti-phosphorylated S129 alpha-synuclein 7E2 antibody at 1.0 g/mL in DISCOVERY Goat Ig Block. Detection: goat anti-rabbit-RP with Silver (top panel) or TAMRA (middle panel), or OptiView with TAMRA (HQ-conjugated goat anti-rabbit antibody/anti-HQ-HRP, bottom panel). PGP9.5 antibody EPR4118 at 0.5 g/mL in DISCOVERY Goat Ig Block is stained yellow. Detection: goat anti-rabbit-AP with Dabsyl (yellow). No pre-treatment with CC1 nor protease.

FIG. 16 shows that the black silver stain deposited in skin nerve features by the anti-phosphorylated alpha-synuclein 7E2 antibody and ultraView SISH DNP Detection Kit provided high-contrast visualization of aggregated phosphorylated S129 alpha-synuclein in the background of PGP9.5 stained yellow using EPR4118 antibody and VENTANA® DISCOVERY UltraMap anti-Rb Alkaline Phosphatase and QM-DABSYL. Tissue sample is skin sample from a subject with PD (top panel), and skin sample form a normal subject (non-PD) (bottom panel).

FIG. 17A shows the immunohistochemical detection of phosphorylated S129 alpha-synuclein (black/silver stain) and PGP9.5 (yellow) in nerve features of a large blood vessel in a skin sample from a subject with PD.

FIG. 17B shows the immunohistochemical detection of phosphorylated S129 alpha-synuclein (black/silver stain) and PGP9.5 (yellow) in nerve features of a small nerve bundle in a skin sample from a subject with PD.

FIG. 17C shows the immunohistochemical detection of phosphorylated S129 alpha-synuclein (black/silver stain) and PGP9.5 (yellow) in nerve features of eccrine sweat glands in a skin sample from a subject with PD.

FIG. 17D shows the immunohistochemical detection of phosphorylated S129 alpha-synuclein (black/silver stain) and PGP9.5 (yellow) in nerve features of a large nerve bundle in a skin sample from a subject with PD.

FIG. 17E shows the immunohistochemical detection of phosphorylated S129 alpha-synuclein (black/silver stain) and PGP9.5 (yellow) in nerve features of arrector pili muscles in a skin sample from a subject with PD.

FIG. 18 shows examples of diffuse granular and discrete types of silver phosphorylated S129 alpha-synuclein stain that could be observed in skin nerve bundles from a non-PD control subject (top panels) and PD subject (bottom panels). Sections of scalp biopsies were stained using phosphorylated S129 alpha-synuclein and PGP9.5 silver/yellow dual IHC assay without protease or phosphatase treatments (Protocol 1). Structures stained yellow are nerves expressing PGP9.5.

FIG. 19 shows similar circumferential neuronal staining patterns between granular type phosphorylated S129 alpha-synuclein (top panel) and myelin basic protein (bottom panel), which suggests potential localization of diffuse pS129 alpha-synuclein in Schwann cells.

FIG. 20A shows that staining of phosphorylated S129 alpha-synuclein by the 11A5 antibody (0.4 g/mL) without CC1 treatment (top panel) was decreased after treatment with ULTRA CC1 for 32 minutes at 100° C. (bottom panel).

FIG. 20B shows that staining of phosphorylated S129 alpha-synuclein by the 7E2 antibody (0.08 g/mL) without CC1 treatment (top panel) was decreased after treatment with ULTRA CC1 for 32 minutes at 100° C. (bottom panel).

FIG. 20C shows that staining of phosphorylated S129 alpha-synuclein by the 3G2 antibody (0.08 g/mL) without CC1 treatment (top panel) was decreased after treatment with ULTRA CC1 for 32 minutes at 100° C. (bottom panel).

FIG. 20D shows that staining of phosphorylated S129 alpha-synuclein by the MJF-R13 antibody (0.4 g/mL) without CC1 treatment (top panel) was decreased after treatment with ULTRA CC1 for 32 minutes at 100° C. (bottom panel).

FIG. 20E shows that staining of phosphorylated S129 alpha-synuclein by the P-Syn/81A antibody (2.0 g/mL) without CC1 treatment (top panel) was decreased after treatment with ULTRA CC1 for 32 minutes at 100° C. (bottom panel).

FIG. 21A shows treatment with VENTANA® Protease 2 for 4 minutes (right panel) led to a moderate increase (compared to no treatment; left panel) in the discrete type of phosphorylated S129 alpha-synuclein (detected with 7E2 antibody clone) stain in skin sections from subjects with PD. Treatment with VENTANA® Protease 2 for 4 minutes also dramatically decreased the granular type of phosphorylated S129 alpha-synuclein stain and PGP9.5 stain in skin sections from subjects with PD.

FIG. 21B shows treatment with VENTANA® Protease 2 for 4 minutes (right panel) significantly decreased (compared to no treatment; left panel) the phosphorylated S129 alpha-synuclein (detected with 7E2 antibody clone) stain in skin sections from non-PD subjects that could be mistaken as the discrete type.

FIG. 22 shows the effect of Protease 3 treatment for 4 minutes (middle panel) or 12 minutes (bottom panel) on staining using phosphorylated S129 alpha-synuclein antibody in skin from a subject with PD compared to no protease treatment (top panel). Phosphorylated S129 alpha-synuclein stained black. 7E2 antibody clone at 1.0 g/mL in DISCOVERY Goat Ig Block (detection: goat anti-rabbit-HRP with Silver). PGP9.5 antibody EPR4118 at 0.5 g/mL in DISCOVERY Goat Ig Block stained yellow (detection: goat anti-rabbit-AP with Dabsyl). No pre-treatment with CC1 nor protease.

FIG. 23A shows that alkaline phosphatase (purified calf intestinal alkaline phosphatase from NEB) pre-treatment at pH9 for 8 minutes (bottom panel) removes diffuse granular phosphorylated alpha-synuclein staining without affecting staining of the dense discrete type normally observed in tissue that was not treated with alkaline phosphatase (top panel).

FIG. 23B shows that alkaline phosphatase (purified calf intestinal alkaline phosphatase from NEB) pre-treatment at pH9 for 24 minutes (top panel) or 40 minutes (bottom panel) removes diffuse granular phosphorylated alpha-synuclein staining without affecting staining of the dense discrete type normally observed in tissue that was not treated with alkaline phosphatase.

FIG. 24 shows that alkaline phosphatase (recombinant alkaline phosphatase from Roche; 30 g/mL) treatment at pH8 for 8 minutes resulted in decreased staining of discrete phosphorylated S129 alpha-synuclein (bottom panel) compared to no AP treatment (top panel) or AP treatment at pH10 (middle panel).

FIG. 25 shows that pretreatment with alkaline phosphatase followed by Protease 3 eliminates diffuse granular phosphorylated alpha-synuclein staining without significant loss of dense discrete staining.

FIG. 26 shows that PGP9.5 detection using EPR4118 antibody (1.0 g/mL) was enhanced with increasing durations (0 minutes—top panel; 16 minutes—middle panel; or 64 minutes—bottom panel) of antigen retrieval using ULTRA CC1 bulk solution.

FIG. 27 shows that antigen retrieval with ULTRA CC2 for 16 minutes at 100° C. yielded enhancement in PGP9.5 staining using EPR4118 antibody.

FIG. 28 shows an overview of two protocols for simultaneous detection of alpha-synuclein (aSyn) and PGP9.5 for Parkinson's disease characterization in skin samples.

FIG. 29 shows individual value plot showing percentages of nerve features containing phosphorylated S129 alpha-synuclein staining in 4 μm sections of scalp biopsy from subjects with PD and normal, non-PD subjects. A cohort of 15 PD and 9 non-PD control scalp skin samples were stained with two different phosphorylated S129 alpha-synuclein and PGP9.5 silver/yellow dual IHC assay protocols: without Ree (Protocol 1) and with (Protocol 2) protease/phosphatase.

FIG. 30A shows individual value plots showing percentages of nerve features with phosphorylated S129 alpha-synuclein stain of discrete types in 4 μm sections of scalp biopsy from subjects with PD and normal, non-PD subjects. A cohort of 15 PD and 7 non-PD control scalp skin samples were stained with phosphorylated S129 alpha-synuclein and PGP9.5 silver/yellow dual IHC assay protocols without (Protocol 1) and with (Protocol 2) protease/phosphatase and also with protease-resistant alpha-synuclein DAB IHC assay developed by pRED/Prothena.

FIG. 30B shows individual value plots showing percentages of nerve features with phosphorylated S129 alpha-synuclein stain of diffuse granular types in 4 μm sections of scalp biopsy from subjects with PD and normal, non-PD subjects. A cohort of 15 PD and 7 non-PD control scalp skin samples were stained with phosphorylated S129 alpha-synuclein and PGP9.5 silver/yellow dual IHC assay protocols without (Protocol 1) and with (Protocol 2) protease/phosphatase and also with protease-resistant alpha-synuclein DAB IHC assay developed by pRED/Prothena.

FIG. 31 shows individual value plot showing percentages of nerve features containing phosphorylated S129 alpha-synuclein staining in 4 μm sections of abdomen skin biopsy from subjects with PD and normal, non-PD subjects. A cohort of 20 PD and 20 non-PD control abdomen skin biopsy samples were stained with two different phosphorylated S129 alpha-synuclein and PGP9.5 silver/yellow dual IHC assay protocols: without (Protocol 1) and with (Protocol 2) protease/phosphatase.

FIG. 32A shows the difference in staining of normal colon tissue by Protocol 1 (top panel) compared to Protocol 2 (bottom panel). Scale bar is 50 m.

FIG. 32B shows the difference in staining of normal colon tissue by Protocol 1 (top panel) compared to Protocol 2 (bottom panel). Scale bar is 50 m.

FIG. 33A shows exemplary staining of submandibular gland tissue from a subject with PD using Protocol 2. Images taken at 10× magnification.

FIG. 33B shows exemplary staining of sigmoid colon tissue from a subject with PD using Protocol 2. Images taken at 20× magnification.

FIG. 33C shows exemplary staining of scalp tissue from a subject with PD using Protocol 2. Images taken at 20× magnification.

FIG. 34 shows that in pediatric skin tissue Protocol 1 (P1; top panel) generated heavy phosphorylated alpha-synuclein silver stain with discrete morphology that was absent with Protocol 2 (P2; bottom panel).

### DETAILED DESCRIPTION

The disclosure involves methods and compositions for accurate and definitive determination of Parkinson's disease (PD) in otherwise undiagnosed subjects with PD, and subjects at risk of or suspected of having Parkinson's Disease. The methods and compositions might also be used to exclude Parkinson's Disease in patients with movement disorders that are sometimes associated with Parkinson's disease-like symptoms, including, but not restricted to, progressive supranuclear palsy, multiple system atrophy, viral parkinsonism, essential tremor, drug- or toxin-induced parkinsonism, post-traumatic parkinsonism, arteriosclerotic parkinsonism, parkinsonism-dementia complex of Guam, cortical basal ganglionic degeneration, or normal pressure hydrocephalus. The disclosed methods include contacting a biological sample comprising at least one nerve feature from the subject with a primary antibody capable of binding phosphorylated alpha-synuclein. By detecting whether the primary antibody capable of binding phosphorylated alpha-synuclein localizes within the nerve feature of the sample, a determination can be made regarding whether a living subject has PD. Subjects may include patients who are pre-symptomatic for PD and those that are in the early stages of disease when accurate diagnosis of PD is currently not routinely possible. In addition, post-mortem analysis can be conducted with biological samples collected after death.

As used herein, the term “biological sample,” includes samples which can be tested by the methods and kits of the present disclosure and includes human and animal body, fixed or frozen, tissue specimens, and fixed cell specimens. The term “tissue” refers to a collection of interconnected cells that perform a similar function within an organism. Biological samples can include samples from a healthy or an apparently healthy human subject or from a human subject affected by or suspected of being affected by a condition or disease to be diagnosed or treated, such as Parkinson's Disease. A biological sample can be a sample obtained from any organ or tissue (including a biopsy such as a tumor biopsy). Also, the samples may be from deceased patients, such as a sample taken during an autopsy. Biological samples can also include cytology samples (in some examples, cytology samples can be sourced from tissues, such as tissue sections from skin tissue, colon tissue, submandibular gland tissue, or brain). In other examples, a sample may be a cell, or cell pellet prepared from a biological sample obtained from a subject.

In some examples, biological samples can include normal or cancerous tissue, for example, skin tissue (from for example, the scalp, abdomen or trunk of a subject), colon tissue, submandibular gland tissue, brain tissue, olfactory bulb, lung tissue, ovarian tissue, pancreatic tissue, mesothelial tissue, gastrointestinal tissue, head and neck tissue, breast tissue, liver tissue, kidney tissue, prostate tissue, uterine tissue, cerebrospinal fluid (or cells and/or nerve features isolated from cerebrospinal fluid), bone, lung cells, ovarian cells, pancreatic cells or mesothelial cells, colon cells, head and neck cells, breast cells, liver cells, kidney cells, skin cells, prostate cells, uterine cells, bone cells, brain cells, and lung cells. For example, a sample from skin, the colon, submandibular gland, olfactory bulb, a lung, ovary, liver, or pancreas, or other tumor that contains cellular material, can be obtained by surgical excision of all or part of a tumor, by collecting a fine, needle brain biopsy, a fine needle aspirate from a tumor or a punch biopsy, as well as other methods known in the art.

As used herein, the term “nerve feature” refers to nerve cells and tissue structures known to be innervated. For example, a nerve feature can be part of a nerve cell, a nerve cell, a part of a former nerve cell, a former nerve cell, or both a nerve cell and a former nerve cell, and/or parts thereof. In some of the methods, the nerve feature is adjacent to a nerve cell, for example a former nerve cell adjacent a healthy nerve cell. Other types of nerve features include tissue structures known to be innervated including, but are not limited to: unmyelinated or thinly myelinated intraepidermal nerve fibers that are widely distributed in dermis; adrenergic, noradrenergic, cholinergic sympathetic fibers or vasodilatory peptidergic fibers that innervate autonomic structures including, but not limited to, sweat glands, hair follicles, arrectores pilorum muscle, and blood vessels. In some methods, a nerve feature can comprise components of former nerve cells or nerve cells that are necrotic or apoptotic.

The term “primary antibody”, in context of the present disclosure, refers to an antibody binding agent, e.g. a whole antibody molecule, a fragment or a derivative of said molecule (e.g. a conjugate comprising an antibody or a polymerized antibody), that specifically binds to a “target” (for example, phosphorylated alpha-synuclein or a component of a nerve feature), more specifically to a single unit of the target of a sample (e.g. an epitope of the target molecule). In some of the methods disclosed herein, the primary antibody can be an antibody capable of binding phosphorylated alpha-synuclein at residue S129 (pS129 aSyn). In some of the methods disclosed herein, the primary can be an antibody capable of binding a component of a nerve feature. Primary antibodies are more fully described under the heading “Antibodies” in the following disclosure.

In order to detect whether the primary antibody localizes within a nerve feature, the sample is contacted with the antibody and the sample is examined for the presence of the antibody. Determination of the localization of the primary antibody can be accomplished, for example, when the primary antibody is labeled with a detectable label. A detection of the label in the sample corresponds to the localization of the antibody within the nerve feature.

Some of the methods disclosed herein further include contacting the biological sample from the subject with a second primary antibody capable of binding a protein other than phosphorylated alpha-synuclein within the nerve feature. The use of the second primary antibody allows for the determination of the localization of both the first primary antibody and the second primary antibody within the nerve feature. The determination of PD can be accurately and definitively determined upon the detection of the co-localization of both antibodies within the nerve feature.

The second primary antibody may be, for example, selected from an antibody capable of binding a protein that is a component of the nerve feature, and can for example be selected from one of the following: ubiquitin C-terminal hydrolase L1 (PGP9.5, UCHL1, NDGOA; PARK5; PGP95; SPG79; Uch-L1; HEL-117; PGP 9.5; HEL-S-53), RNA binding fox-1 homolog 3 (RBFOX3, FOX3, NEUN, FOX-3, HRNBP3), microtubule associated protein 2 (MAP2, MAP2A, MAP2B, MAP2C), 160 kDa neurofilament medium (NEFM, NFM, NEF3, NF-M), 200 kDa neurofilament heavy (NEFH, NFH, CMT2CC), synaptophysin (SYP, MRX96, MRXSYP), and discs large MAGUK scaffold protein 4 (DLG4, DLGH4, PSD-95, PSD95, SAP-90, SAP90, SAP90A).

In some of the methods disclosed herein, the primary antibody capable of binding phosphorylated alpha-synuclein and the primary antibody capable of binding to the nerve feature are from the same host species, and the host species can be a mouse or a rabbit. In some methods, the primary antibody capable of binding phosphorylated alpha-synuclein and the primary antibody capable of binding to the nerve feature are from different host species, such as mouse or rabbit.

The terms “localization” and “co-localization” refer to the location of a target protein or target proteins within the same nerve feature. In some methods, co-localization of proteins within in a nerve feature is determined when signals independently indicative the proteins are identified in the nerve feature when compared to background for each signal. A signal indicative of a peptide that is outside a nerve feature is not indicative of a protein localized within a nerve feature. As one example, a signal indicative of phosphorylated alpha-synuclein localized in a nerve feature is a signal at one, two, three or four standard deviations higher than a background signal, or at least 1-fold, at least 2 fold, at least 3 fold, at least 4 fold, at least 5 fold, at least 6 fold, at least 7 fold, at least 8 fold, at least 9 fold, or at least 10 fold higher that background signal.

Methods for determining the localization and co-localization of antibodies include, for example, histochemical analysis. As used herein, the terms “immunohistochemistry” or “IHC” refer to method of determining the presence or distribution of an antigen in a fixed or frozen sample by detecting interaction of the antigen with a specific binding agent, such as an antibody, within the sample. Accordingly, the disclosure provides methods for determining whether a subject has Parkinson's Disease (PD) by using a fixed or frozen section of a biological sample from a subject suspected of having PD. Phosphorylated alpha-synuclein localized within a nerve feature in the section may be detected and the subject may be diagnosed with PD when phosphorylated alpha-synuclein localized within a nerve feature. Methods of the disclosure include contacting the section with a first primary antibody capable of binding phosphorylated alpha-synuclein. The method may also include contacting the section with a second primary antibody capable of binding the nerve feature.

Some of the methods disclosed herein may also include an automated histochemical staining platform, such as an automated IHC/ISH slide stainer. Automated IHC/ISH slide stainers typically include at least: reservoirs of the various reagents used in the staining protocols, a reagent dispense unit in fluid communication with the reservoirs for dispensing reagent to onto a slide, a waste removal system for removing used reagents and other waste from the slide, and a control system that coordinates the actions of the reagent dispense unit and waste removal system. In addition to performing staining steps, many automated slide stainers can also perform steps ancillary to staining (or are compatible with separate systems that perform such ancillary steps), including: slide baking (for adhering the sample to the slide), dewaxing (also referred to as deparaffinization), antigen retrieval, counterstaining, dehydration and clearing, and coverslipping. Prichard, Overview of Automated Immunohistochemistry, Arch Pathol Lab Med., Vol. 138, pp. 1578-1582 (2014), incorporated herein by reference in its entirety, describes several specific examples of automated IHC/ISH slide stainers and their various features, including the intelliPATH FLX® (Biocare Medical), WAVE® (Celerus Diagnostics), DAKO OMNIS® and DAKO AUTOSTAINER LINK® 48 (Agilent Technologies), BENCHMARK® (Ventana Medical Systems, Inc.), Leica BOND®, and Lab Vision™ Autostainer (Thermo Scientific) automated slide stainers. Additionally, Ventana Medical Systems, Inc. is the assignee of a number of United States patents disclosing systems and methods for performing automated analyses, including U.S. Pat. Nos. 5,650,327, 5,654,200, 6,296,809, 6,352,861, 6,827,901, and 6,943,029, and U.S. Published Patent Application Nos.: 20030211630 and 20040052685, each of which is incorporated herein by reference in its entirety. Commercially-available staining units typically operate on one of the following principles: (1) open individual slide staining, in which slides are positioned horizontally and reagents are dispensed as a puddle on the surface of the slide containing a tissue sample (such as implemented on the DAKO AUTOSTAINER LINK® 48 (Agilent Technologies) and intelliPATH® (Biocare Medical) stainers); (2) liquid overlay technology, in which reagents are either covered with or dispensed through an inert fluid layer deposited over the sample (such as implemented on VENTANA® BenchMark and DISCOVERY® stainers); (3) capillary gap staining, in which the slide surface is placed in proximity to another surface (which may be another slide or a coverplate) to create a narrow gap, through which capillary forces draw up and keep liquid reagents in contact with the samples (such as the staining principles used by DAKO TECHMATE®, Leica BOND®, and DAKO OMNIS® stainers). Some iterations of capillary gap staining do not mix the fluids in the gap (such as on the DAKO TECHMATE® and the Leica BOND®). In variations of capillary gap staining termed dynamic gap staining, capillary forces are used to apply sample to the slide, and then the parallel surfaces are translated relative to one another to agitate the reagents during incubation to effect reagent mixing (such as the staining principles implemented on DAKO OMNIS® slide stainers (Agilent)). In translating gap staining, a translatable head is positioned over the slide. A lower surface of the head is spaced apart from the slide by a first gap sufficiently small to allow a meniscus of liquid to form from liquid on the slide during translation of the slide. A mixing extension having a lateral dimension less than the width of a slide extends from the lower surface of the translatable head to define a second gap smaller than the first gap between the mixing extension and the slide. During translation of the head, the lateral dimension of the mixing extension is sufficient to generate lateral movement in the liquid on the slide in a direction generally extending from the second gap to the first gap. See WO 2011-139978 A1. It has recently been proposed to use inkjet technology to deposit reagents on slides. See WO 2016-170008 A1. This list of staining technologies is not intended to be comprehensive, and any fully or semi-automated system for performing biomarker staining may be incorporated into the histochemical staining platform.

### Detection of Antibody Binding

In some of the methods disclosed herein, step (a) further comprises contacting the sample with a first secondary antibody having a first label conjugated thereto, wherein the first secondary antibody is immunoreactive with the primary antibody capable of binding phosphorylated alpha-synuclein. In some of the methods disclosed herein, the methods comprise contacting the sample with a set of reagents reactive with the first label of the first secondary antibody to generate a first detectable signal in proximity to phosphorylated alpha-synuclein in the sample.

In some of the methods disclosed herein, the method further comprises contacting the sample with a second secondary antibody having a second label detectable conjugated thereto, wherein the second secondary antibody is immunoreactive with the primary antibody capable of binding the nerve feature. In some of the methods disclosed herein, the methods comprise contacting the sample with a set of reagents reactive with the second label of the second secondary antibody to generate a second detectable signal in proximity to the nerve feature in the sample. In some of the methods disclosed herein, the first detectable signal and the second detectable signal are different. In certain methods as disclosed herein, the first detectable signal is silver stain and the second detectable signal is QM-Dabsyl or Fast-Red.

The term “secondary antibody,” in context of the present disclosure, refers to an antibody binding agent, e.g. a whole antibody molecule, a fragment or a derivative of said molecule (e.g. a conjugate comprising an antibody or a polymerized antibody), that has an antigen binding domain that specifically binds to the primary antibody that is bound to a target antigen. Secondary antibodies can help increase sensitivity and signal amplification due to multiple secondary antibodies binding to a primary antibody. In some embodiments, secondary antibodies can be conjugated to enzymes such as horseradish peroxidase (HRP) or alkaline phosphatase (AP); or fluorescent dyes such as fluorescein isothiocyanate (FITC), rhodamine derivatives, Alexa Fluor dyes; or other molecules to be used in various applications.

In some of the methods disclosed herein, the sample is contacted with at least one protease before being contacted with the primary antibody capable of binding phosphorylated alpha-synuclein. In certain methods as disclosed herein, the methods further comprise contacting the sample with at least one phosphatase. In some of the methods, the sample is contacted with both a protease and a phosphatase. The use of the protease or the protease and the phosphatase may be used according to a method wherein the determination of PD is accomplished with the first primary antibody alone or wherein the determination is accomplished with the use of both the first primary antibody and the second primary antibody. In some of the methods as disclosed herein, the use of the phosphatase enhances detection of phosphorylated alpha-synuclein unique to subjects with Parkinson's disease by removing phosphorylated alpha-synuclein normally seen in subjects without Parkinson's disease. The use of the protease enhances detection of phosphorylated alpha-synuclein by improving accessibility of phosphorylated alpha-synuclein to primary antibody.

In some of the methods disclosed herein, before contacting the sample with the primary antibody capable of binding to the nerve feature, the method comprises denaturing the immunocomplexes in the sample by, for example incubating the sample at 100° C. for at least 15 minutes. In certain methods, denaturing can occur at about 80° C. to at least about 110° C. for about 5 minutes to at least about 60 minutes. For example, denaturing can occur at about 80° C., about 85° C., about 90° C., about 95° C., about 100° C., about 105° C., or about 110° C., for at least about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 25 minutes, about 30 minutes, about 35 minutes, about 40 minutes, about 45 minutes, or about 48 minutes, or about 50 minutes, or about 55 minutes or at least about 60 minutes.

As used herein, the terms “protease” or “peptidase” or “proteinase” refer to an enzyme that performs proteolysis or protein catabolism by hydrolysis of peptide bonds. Proteases can be classified into seven broad groups depending on the catalytic residue. For example, serine proteases (using serine alcohol), cysteine proteases (using a cysteine thiol), threonine proteases (using a threonine secondary alcohol), aspartic proteases (using an aspartate carboxylic acid), glutamic proteases (using a glutamate carboxylic acid), metalloproteases (using a metal, usually zinc), and asparagine peptide lyases (using an asparagine to perform an elimination reaction). Examples of proteases can include, but are not limited to: trypsin, chymotrypsin, enterokinase, endoproteinase GluC, proteinase K, thrombin, Factor Xa, bromelain, alkaline proteases (for example, alkaline VIII protease), papain, collagenase, dispase, pepsin, cathepsin D, and carboxypeptidase A. Examples of commercially available proteases can include, but are not limited to: VENTANA® Protease 1 (P/N 760-2018), VENTANA® Protease 2 (P/N 760-2019), or VENTANA® Protease 3 (P/N 760-2020). Protease incubation times can be determined by a person skilled in the art, but incubation times can range from about 2 minutes or less to at least about 20 minutes, and temperatures can range from about 15° C. or less to at least about 45° C. For example, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or at least about 20 minutes, at about 15° C., at about 20° C., at about 25° C., at about 30° C., at about 35° C., at about 36° C., at about 37° C., at about 38° C., at about 39° C., or at least about 40° C. In certain examples, the incubation can be for about 4 minutes at about 36° C., or about 4 minutes at 40° C., or about 4 minutes and 30 seconds at 40° C., or about 5 minutes at 40° C., or about 12 minutes at about 36° C.

As used herein, the term “phosphatase” refers to an enzyme that uses water to cleave a phosphoric acid monoester into a phosphate ion and an alcohol (phosphatases remove phosphate groups from molecules). Examples of phosphatases can include, but are not limited to, alkaline phosphatases, acid phosphatases, phosphoprotein phosphatase 1 (PP1), phosphoprotein phosphatase 2A (PP2A), phosphoprotein phosphatase 2B (PP2B), phosphoprotein phosphatase 2C (PP2C), and lambda protein phosphatase. Examples of commercially available phosphatases can include, but are not limited to: bovine alkaline phosphatase from Pichia pastoris (Roche P/N 3359123001). Phosphatase incubation times can be determined by a person skilled in the art, but incubation times can range from about 5 minutes to at least about 12 hours, and temperatures can range from about 20° C. to at least about 50° C. For example, about 8 minutes, about 9 minutes, about 10 minutes, about 15 minutes, about 20 minutes, about 25 minutes, about 30 minutes, about 45 minutes, about 60 minutes, about 90 minutes, about 2 hours, about 3 hours, about 4 hours, about 5 hours, about 6 hours, about 7 hours, about 8 hours, about 9 hours, about 10 hours, about 11 hours, or at least about 12 hours, at about 20° C., about 21° C., about 22° C., about 23° C., about 24° C., about 25° C., about 30° C., at about 36° C., at about 37° C., at about 38° C., at about 39° C., or at least about 40° C. In certain examples, the incubation can be for about 2 hours at about 36° C., or about 10 minutes at 37° C., or about 60 minutes at 37° C., or about 45 minutes at 37° C., or about 60 minutes at about 37° C.

In some of the methods disclosed herein, incubation times vary and depend on the temperature of the incubation. Incubation periods for substantially complete antibody binding, color development, and other steps of the method are well known. Many of the steps, e.g. antigen unmasking, antibody binding, and color development, are performed at elevated temperatures of at least about 35° C., preferably from about 40° to about 45° C., or up to 100° C. for a time of about 4 minutes to about 90 minutes. For example, in certain methods, the primary antibodies can be incubated with the samples at 36° C. for 32 minutes or they can be incubated at about 22° C. for 20 minutes. In another example, the protease can be incubated with the samples at 36° C. for 12 minutes or the protease can be incubated with the samples at 36° C. for 4 minutes. In yet another example, antigen unmasking can be performed at least about 100° C. for about 30-90 minutes. However, with the exception of steps dependent on enzyme activity (antigen unmasking and color development), most steps can also be performed at temperatures as low as 4° C., if the incubation period is appropriately increased.

When the antibody staining reagent is specific for an antigen that requires unmasking, the tissue section can be treated with a proteolytic enzyme or protease prior to addition of the primary antibody. The proteolytic enzyme or protease can be added to the evaporation inhibitor liquid covering the tissue section and sinks through the evaporation inhibitor liquid to the tissue section below. Following a sufficient period of incubation for antigen unmasking, for example about 4 to about 30 minutes, the slide can be washed and the evaporation inhibitor liquid can be reapplied.

In some of the methods, prior to antibody application, a sufficient amount of a protease solution to cover the tissue section, conveniently about 100-200 μL, can be applied to paraffin-embedded, formalin-fixed tissue. Treatment with a protease can help expose the antigens to the labeling reagents, allowing for more accurate results. In some of the methods, the incubation period is sufficient to disrupt crosslinking, but not so long that the antigens are destroyed. The period depends on the temperature, enzyme concentration, tissue thickness, tissue type and amount of time in formalin. An appropriate time can be readily determined by one of skill in the art. For example, about 4 minutes at about 36° C., or about 4 minutes at 40° C., or about 4 minutes and 30 seconds at 40° C., or about 5 minutes at 40° C., or about 12 minutes at about 36° C. can be effective. Following incubation, the protease solution can be washed off, and the next reagent in the staining/labeling process is applied. When these ranges are not observed, over and/or under digestion of the tissue by the protease is likely to occur, which can result in destroyed antigens/masked antigens, respectively. Following the incubation, the slide can be rinsed.

### Antibodies

As used herein, the term “antibody” refers to a polypeptide ligand comprising at least a light chain or heavy chain immunoglobulin variable region which specifically recognizes and binds an epitope of an antigen. Antibodies include at least a light chain or heavy chain immunoglobulin variable region or immunoglobulin-like molecules (including by way of example and without limitation, IgA, IgD, IgE, IgG and IgM, combinations thereof, and similar molecules produced during an immune response in any vertebrate, for example, in mammals such as humans, goats, rabbits and mice), as well as non-mammalian species, such as shark immunoglobulins. Antibodies include monoclonal antibodies, polyclonal antibodies, or fragments of antibodies as well as others known in the art. In some examples, an antibody is labeled with a detectable label, such as an enzyme or fluorophore. Antibody also includes antibody fragments that specifically bind to a molecule of interest (or a group of highly similar molecules of interest) to the substantial exclusion of binding to other molecules.

The term “antibody” also includes an antigen binding fragment of a naturally occurring or recombinant antibody. Non-limiting examples of binding fragments encompassed within the term antibody include Fab, (Fab′)₂, Fv, and single-chain Fv (scFv). Fab is the fragment that contains a monovalent antigen-binding fragment of an antibody molecule produced by digestion of whole antibody with the enzyme papain to yield an intact light chain and a portion of one heavy chain or equivalently by genetic engineering. Fab′ is the fragment of an antibody molecule obtained by treating whole antibody with pepsin, followed by reduction, to yield an intact light chain and a portion of the heavy chain; two Fab′ fragments are obtained per antibody molecule. (Fab′)₂ is the fragment of the antibody obtained by treating whole antibody with the enzyme pepsin without subsequent reduction or equivalently by genetic engineering. F(Ab′)₂ is a dimer of two FAb′ fragments held together by disulfide bonds. Fv is a genetically engineered fragment containing the variable region of the light chain and the variable region of the heavy chain expressed as two chains. Single chain antibody (“SCA”) is a genetically engineered molecule containing the variable region of the light chain, the variable region of the heavy chain, linked by a suitable polypeptide linker as a genetically fused single chain molecule. Methods of making these fragments are routine in the art.

Antibodies and antibody binding fragments that have a binding constant for the molecule of interest (i.e., phosphorylated alpha-synuclein and proteins endogenous in a nerve feature) that is at least 10³ M⁻¹ greater, at least 10⁴ M⁻¹ greater or at least 10⁵ M⁻¹ greater than a binding constant for other molecules in a biological sample. In some examples, an antibody has a high binding affinity for phosphorylated alpha-synuclein or PGP9.5 such as a binding affinity of at least about 1×10⁻⁸ M, at least about 1.5×10⁻⁸, at least about 2.0×10⁻⁸, at least about 2.5×10⁻⁸, at least about 3.0×10⁻⁸, at least about 3.5×10⁻⁸, at least about 4.0×10⁻⁸, at least about 4.5×10⁻⁸, or at least about 5.0×10⁻⁸ M. In certain embodiments, an antibody that binds to phosphorylated alpha-synuclein or PGP9.5 has a dissociation constant (Kd) of ≤10⁴ nM, ≤10³ nM, ≤100 nM, ≤10 nM, ≤1 nM, ≤0.1 nM, ≤0.01 nM, or ≤0.001 nM, or ≤0.0001 nM, or ≤0.00001 nM (e.g., 10⁻⁴ M or to 10⁻⁶ M, e.g., from 10⁻⁷ M to 10⁻⁹ M, e.g., from 10⁻¹⁰ M to 10⁻¹² M, or e.g., from 10⁻¹³ M to 10⁻¹⁵ M). In one embodiment, Kd can be measured by a radiolabeled antigen binding assay (RIA) performed with the Fab version of an antibody of interest and its antigen. In another example, Kd can be measured using a label-free optical scanner for microarray detection based on polarization-modulated oblique-incidence reflectivity difference (OI-RD). In yet another example, Kd can be measured using surface plasmon resonance assays using a BIACORE®-2000 or a BIACORE®-3000 (BIAcore, Inc., Piscataway, N.J.) at 25° C. with immobilized antigen CM5 chips at about 10 response units (RU).

Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds phosphorylated alpha-synuclein will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

Several antibodies to alpha-synuclein (also referred to as, SNCA, PD1; NACP; PARK1; PARK4; aSyn) and phosphorylated alpha-synuclein are known. Alpha-synuclein is a member of the synuclein family, which also includes beta- and gamma-synuclein. Synucleins are expressed in the brain and alpha- and beta-synuclein inhibit phospholipase D2 selectively. Alpha-synuclein may serve to integrate presynaptic signaling and membrane trafficking. Mutations in alpha-synuclein have been implicated in the pathogenesis of Parkinson disease. Alpha-synuclein peptides are a major component of amyloid plaques in the brains of patients with Alzheimer's disease. In certain methods, phosphorylated alpha-synuclein can be detected. Alpha-synuclein sequences are publically available, for example from GenBank™ sequence database (e.g., amino acids Accession No. NP_000336.1 encoded by Accession No. NM_000345.3). One of ordinary skill in the art can identify additional alpha-synuclein nucleic acid and protein sequences, including alpha-synuclein variants and isoforms. Antibodies for detecting alpha-synuclein are known in the art, and can include, but are not limited to: rabbit monoclonal antibody clones 7E2, and 3G2 against phospho-S129 alpha-synuclein generated by Roche Diagnostics GmbH CPS R&D Early Development & Reagent Design (DXREAA) with a phospho-peptide corresponding to human alpha-synuclein residues 122-135 as immunogen. Additional monoclonal antibodies against phosphorylated S129 alpha-synuclein (aSyn) can be purchased, for example, from Abcam (clones MJF-R13 (8-8), P/N ab168381 and P-syn/81A, P/N ab184674) or WAKO (clone pSyn #64, P/N 015-25191). Mouse monoclonal antibody clone LB509 against alpha-synuclein regardless of S129 phosphorylation status can be purchased from Abcam (P/N ab27766). Anti-alpha-synuclein mouse monoclonal antibody clone 5C12 (S129 phosphorylation independent; ATCC® No. PTA-9197) and anti-phosphorylated S129-alpha-synuclein mouse monoclonal antibody clone 11A5 (ATCC® No. PTA-8222) from Prothena Corporation may also be used. Also see Table 10 below.

An example of an anti-phosphorylated S129 alpha-synuclein antibody is the rabbit monoclonal antibody clone 7E2, which was generated by Roche Diagnostics GmbH CPS R&D Early Development & Reagent Design (DXREAA) with a phospho-peptide corresponding to human alpha-synuclein residues 122-135 as immunogen. The 7E2 antibody or antigen binding fragment thereof comprises a light chain (LC) variable region comprising the amino acid sequence of SEQ ID NO:01. The 7E2 antibody or antigen binding fragment thereof comprises a heavy chain (HC) variable region comprising the amino acid sequence of SEQ ID NO:05

SEQ ID NO: 01 (7E2 light chain):
AQVLTQTPSPVSAAVGGTVTISCQSSQSVYNNNNLVWYQQKPGQPPKQVIYKASKVASGVPSRFKGSGS
GTQFTLTISDLECDDAATYYCLGGYSGDIYTFGGGTEVVVKGDPVAPTVLIFPPAADQVATGTVTIVCV
ANKYFPDVTVTWEVDGTTQTTGIENSKTPQNSADCTYNLSSTLTLTSTQYNSHKEYTCKVTQGTTSVVQS
FNRGDC
SEQ ID NO: 02 7E2 LC CDR1:
QSVYNNNN
SEQ ID NO: 03 7E2 LC CDR2:
KASKVAS
SEQ ID NO: 04 7E2 LC CDR3:
LGGYSGDIYT
SEQ ID NO: 05 (7E2 heavy chain):
CQSVEESGGRLVTPGTPLTLTCTASGFTISSYHMSWVRQAPGKGLEWIGYISTSGNIYYASWAKGRFTI
SKTSSTTVDLRMTSLTTEDTATYFCARLGIATGYSFWGHGTLVTVSSGQPKAPSVFPLAPCCGDTPSST
VTLGCLVKGYLPEPVTVTWNSGTLTNGVRTFPSVRQSSGLYSLSSVVSVTSSSQPVTCNVAHPATNTKVD
KTVAPSTCSKPTCPPPELLGGPSVFIFPPKPKDTLMISRTPEVTCVVVDVSQDDPEVQFTWYINNEQVRT
ARPPLREQQFNSTIRVVSTLPIAHQDWLRGKEFKCKVHNKALPAPIEKTISKARGQPLEPKVYTMGPPRE
ELSSRSVSLTCMINGFYPSDISVEWEKNGKAEDNYKTTPAVLDSDGSYFLYNKLSVPTSEWQRGDVFTCS
VMHEALHNHYTQKSISRSPGK
SEQ ID NO: 06
7E2 HC CDR1:
GFTISSYHMS
SEQ ID NO: 07
7E2 HC CDR2:
ISTSGNI
SEQ ID NO: 08
7E2 HC CDR3:
ARLGIATGYSF

In some embodiments, there are provided antibodies having a light chain variable region that has an amino acid sequence that shares at least 90% sequence identity with an amino acid sequence represented by SEQ ID NO:01. In a further embodiment, there are provided antibodies having a heavy chain variable region that has an amino acid sequence that shares at least 90% sequence identity with an amino acid sequence represented by SEQ ID NO:05.

Another example of an anti-phosphorylated S129 alpha-synuclein antibody is the rabbit monoclonal antibody clone 3G2, which was generated by Roche Diagnostics GmbH CPS R&D Early Development & Reagent Design (DXREAA) with a phospho-peptide corresponding to human alpha-synuclein residues 122-135 as immunogen. The 3G2 antibody or antigen binding fragment thereof comprises a light chain (LC) variable region comprising the amino acid sequence of SEQ ID NO:09. The 3G2 antibody or antigen binding fragment thereof comprises a heavy chain (HC) variable region comprising the amino acid sequence of SEQ ID NO:13.

SEQ ID NO: 09 (3G2 light chain):
AQVLTQTPSPVSAAVGGTVTISCQSSQSVYNNNNLVWFQKKPGQPPKQLIYKASKVASGVPSRFKGSGS
GTQFTLTISDLECDDAATYYCLGGYSGDIYTFGGGTEVVVKGDPVAPTVLIFPPAADQVATGTVTIVCV
ANKYFPDVTVTWEVDGTTQTTGIENSKTPQNSADCTYNLSSTLTLTSTQYNSHKEYTCKVTQGTTSVVQS
FNRGDC
SEQ ID NO: 10 3G2 LC CDR1:
QSVYNNNN
SEQ ID NO: 11 3G2 LC CDR2:
KASKVAS
SEQ ID NO: 12 3G2 LC CDR3:
LGGYSGDIYT
SEQ ID NO: 13 (3G2 heavy chain):
QEQLKESGGGLVTPGGTLTLTCTASGFTISSYHMSWVRQAPGKGLEWIGYISTSGNIYYATWAKGRFTI
SKTSSTTVDLRMTSLTTEDTATYFCARLGIATGYSFWGHGTLVTVSSGQPKAPSVFPLAPCCGDTPSST
VTLGCLVKGYLPEPVTVTWNSGTLTNGVRTFPSVRQSSGLYSLSSVVSVTSSSQPVTCNVAHPATNTKVD
KTVAPSTCSKPTCPPPELLGGPSVFIFPPKPKDTLMISRTPEVTCVVVDVSQDDPEVQFTWYINNEQVRT
ARPPLREQQFNSTIRVVSTLPIAHQDWLRGKEFKCKVHNKALPAPIEKTISKARGQPLEPKVYTMGPPRE
ELSSRSVSLTCMINGFYPSDISVEWEKNGKAEDNYKTTPAVLDSDGSYFLYNKLSVPTSEWQRGDVFTCS
VMHEALHNHYTQKSISRSPGK
SEQ ID NO: 14
3G2 HC CDR1:
GFTISSYHMS
SEQ ID NO: 15 3G2 HC CDR2:
ISTSGNI
SEQ ID NO: 16 3G2 HC CDR3:
ARLGIATGYSF

In some embodiments, there are provided antibodies having a light chain variable region that has an amino acid sequence that shares at least 90% sequence identity with an amino acid sequence represented by SEQ ID NO:09. In a further embodiment, there are provided antibodies having a heavy chain variable region that has an amino acid sequence that shares at least 90% sequence identity with an amino acid sequence represented by SEQ ID NO:13.

The disclosure also provides methods of detecting one or more proteins that are part of nerve features, as defined herein. Such proteins can be detected by an antibody capable of binding a protein selected from: ubiquitin C-terminal hydrolase L1 (PGP9.5, UCHL1, NDGOA; PARK5; PGP95; SPG79; Uch-L1; HEL-117; PGP 9.5; HEL-S-53; and Accession No's. NM_004181.4→NP_004172.2), RNA binding fox-1 homolog 3 (RBFOX3, FOX3, NEUN, FOX-3, HRNBP3; and Accession No's. NM_001082575.2→NP_001076044.1), microtubule associated protein 2 (MAP2, MAP2A, MAP2B, MAP2C; and Accession No's. NM_001039538.1→NP_001034627.1), 160 kDa neurofilament medium (NEFM, NFM, NEF3, NF-M; and Accession No's. NM_005382.2→NP_005373.2), 200 kDa neurofilament heavy (NEFH, NFH, CMT2CC; and Accession No's. NM_021076.3→NP_066554.2), synaptophysin (SYP, MRX96, MRXSYP; and Accession No's. NM_003179.2→NP_003170.1), or discs large MAGUK scaffold protein 4 (DLG4, DLGH4, PSD-95, PSD95, SAP-90, SAP90, SAP90A; and Accession No's. NM_001365.4→NP_001356.1). One of ordinary skill in the art can identify additional PGP9.5, RBFOX3, MAP2, NEFM, NEFH, SYP, and DLG4 nucleic acid and protein sequences, including variants and isoforms. Antibodies for detecting PGP9.5, RBFOX3, MAP2, NEFM, NEFH, SYP, and DLG4 are known in the art. For example antibodies capable of binding PGP9.5, can include, but are not limited to: anti-human PGP9.5 monoclonal antibodies from rabbit (clone EPR4118, P/N ab108986), or mouse (clone 13C/13C4, P/N ab8189), can be purchased from Abcam. A rabbit polyclonal antibody with RTD P/N 760-4434 can be obtained from Cell Marque™.

As used herein, the term “contacting” refers to placement that allows association between two or more moieties, particularly direct physical association, for example both in solid form and/or in liquid form (for example, the placement of a biological sample, such as a biological sample affixed to a slide, in contact with an antigen releasing solution).

As used herein, the term “specifically binds” refers to the binding of agent that preferentially binds to a defined target (such as an antibody to a specific antigen or a nucleic acid probe to a specific nucleic acid sequence). A target can be any molecule for which the presence, location and/or concentration is or can be determined. Examples of target molecules include proteins and nucleic acids. Target molecules are typically detected using one or more conjugates of a specific binding molecule and a detectable label. With respect to an antigen, “specifically binds” refers to the preferential association of an antibody or other ligand, in whole or part, with a specific polypeptide. With respect to a nucleic acid sequence, “specifically binds” refers to the preferential association of a nucleic acid probe, in whole or part, with a specific nucleic acid sequence. A specific binding agent binds substantially only to a defined target. It is recognized that a minor degree of non-specific interaction may occur between a molecule, such as a specific binding agent, and a non-target polypeptide or non-target nucleic acid sequence. Although a selectively reactive antibody binds an antigen, it can do so with low affinity. Antibody to antigen specific binding typically results in greater than 2-fold, such as greater than 5-fold, greater than 10-fold, or greater than 100-fold increase in amount of bound antibody or other ligand (per unit time) to a target polypeptide, as compared to a non-target polypeptide. A variety of immunoassay formats are appropriate for selecting antibodies specifically immunoreactive with a particular protein. For example, solid-phase ELISA immunoassays are routinely used to select monoclonal antibodies specifically immunoreactive with a protein.

A sample including an antigen (such as a target antigen) is incubated with an antibody under conditions permitting antibody-antigen binding. Antibody-antigen binding can be detected by means of a detectable label conjugated to the antibody (direct detection) or by means of a detectable label conjugated to a secondary antibody, which is raised against the primary antibody (e.g., indirect detection). Detectable labels can include, but are not limited to, radioactive isotopes, fluorochromes (such as fluorescein, fluorescein isothiocyanate, and rhodamine), and chromogenic molecules.

As used herein, the term “detect” refers to determining if an agent (such as a signal or particular antigen or protein) is present or absent in a sample. In some examples, this can further include quantification. As used herein, the term “detecting” refers to any method of determining if something exists, or does not exist, such as determining if a target molecule is present in a biological sample. For example, detecting can include using a visual or a mechanical device to determine if a sample displays a specific characteristic. In certain examples, detection refers to visually observing a probe bound to a target, or observing that a probe does not bind to a target. For example, light microscopy and other microscopic means are commonly used to detect chromogenic precipitates for methods described here.

### Labels

As used herein, “detectable label” refers to a molecule or material that can produce a detectable signal (such as visually, electronically or otherwise) that indicates the presence and/or concentration of a target in a sample. When conjugated to a specific binding molecule, the detectable label can be used to locate and/or quantify the target to which the specific binding molecule is directed. Thereby, the presence and/or concentration of the target in a sample can be detected by detecting the signal produced by the detectable label. A detectable label can be detected directly or indirectly, and several different detectable labels conjugated to different specific-binding molecules can be used in combination to detect one or more targets. Multiple detectable labels that can be separately detected can be conjugated to different specific binding molecules that specifically bind different targets to provide a multiplexed assay that can provide detection of the multiple targets in a sample. A detectable signal can be generated by any known or yet to be discovered mechanism including absorption, emission and/or scattering of a photon (including radio frequency, microwave frequency, infrared frequency, visible frequency and ultra-violet frequency photons).

Detectable labels can include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity). Particular examples of detectable labels include: enzymes, such as horseradish peroxidase, alkaline phosphatase, acid phosphatase, glucose oxidase, β-galactosidase or β-glucuronidase; fluorophores, such as fluoresceins, luminophores, coumarins, BODIPY dyes, resorufins, and rhodamines (many additional examples of fluorescent molecules can be found in The Handbook—A Guide to Fluorescent Probes and Labeling Technologies, Molecular Probes, Eugene, OR); nanoparticles, such as quantum dots; metal chelates, such as DOTA and DPTA chelates of radioactive or paramagnetic metal ions like Gd³⁺; and liposomes, for example, liposomes containing trapped fluorescent molecules. Where the detectable label includes an enzyme, a detectable substrate such as a chromogen, a fluorogenic compound, or a luminogenic compound is used in combination with the enzyme to generate a detectable signal (a wide variety of such compounds are commercially available, for example, from Life Technologies, Carlsbad, Calif.).

As used herein, the term “chromogen” refers to a substance capable of conversion to a colored product, such as a pigment or dye. Certain chromogens are electron donors that, when oxidized, become a colored product. Production of a colored product, or the property of becoming insoluble upon chemical conversion, such as by oxidation, make chromogens useful for IHC. Non-limiting examples of chromogenic compounds, include diaminobenzidine (DAB), 4-Chloro-2-methyl-benzenediazonium (Fast Red), nitro blue tetrazolium (NBT), AP Orange, tetramethylbenzidine (TMB), 2,2′-azino-di-[3-ethylbenzothiazoline sulphonate](ABTS), New Fuchsin, iodonitrotetrazolium (INT), tetrazolium blue and tetrazolium violet. More chromogens are known to those skilled in the art, for example, 4-nitrophenylphospate (pNPP), bromochloroindolyl phosphate (BCIP), nitro blue tetrazolium (NBT), AP blue, o-dianisidine, 4-chloronaphthol (4-CN), nitrophenyl-.beta.-D-galactopyranoside (ONPG), o-phenylenediamine (OPD), 5-bromo-4-chloro-3-indolyl-.beta.-galactopyranoside (X-Gal), methylumbelliferyl-.beta.-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue and tetrazolium violet. DAB is a chromogen that produces a brown end product that is highly insoluble in alcohol and other organic solvents. Oxidation of DAB causes polymerization, resulting in the ability to react with osmium tetroxide, and thus increasing its staining intensity and electron density.

Alternatively, an enzyme can be used in a metallographic detection scheme. Metallographic detection methods include using an oxido-reductase enzyme (such as horseradish peroxidase) along with a water soluble metal ion (for example, silver or gold), an oxidizing agent and a reducing agent, again to form a detectable precipitate. Under the proper conditions (i.e., the addition of the label enzyme) will reduce soluble metal ions such as silver (+1) or gold (+3) to a silver or gold atom such that it becomes visible to the eye as a specific dot under a brightfield light microscope.

### Kits

The disclosure also provides a kit comprising: a primary antibody capable of binding phosphorylated alpha-synuclein; and a primary antibody capable of binding to a nerve feature. In some of the kits disclosed herein, the kits further comprise: (a) a first secondary antibody having a first label conjugated thereto, wherein the first secondary antibody is immunoreactive with the primary antibody capable of binding phosphorylated alpha-synuclein; (b) a set of reagents that generates a first detectable single when reacted with the first label of the first secondary antibody; (c) a second secondary antibody having a second label conjugated thereto, wherein the second secondary antibody is immunoreactive with the primary antibody capable of binding the nerve feature; and (d) a set of reagents that generates a second detectable single when reacted with the second label of the second secondary antibody; wherein the first detectable signal and the second detectable signal are different. In some of the kits, the first detectable signal is silver stain and the second detectable signal is QM-Dabsyl or Fast-Red.

As used herein, the term “kit” refers to a packaged combination of one or more vessels, containers, devices or the like holding the necessary reagents for detecting an analyte of interest. The kit is appended with written or computerized instructions for performing the method. The kit may contain a labeled antibody, nucleic acid, ligand, or the like. The kit for detecting an analyte of interest in a biological sample can comprise one or more containers, each container adapted to hold a specific binding member for the analyte of interest, a redox-inactive reductive species, an enzyme label for rendering said reductive species active, a metal ion, and reagents for metal enhancement. In some embodiments, said specific binding member is immobilized on a solid support.

In some of the methods disclosed herein, the primary antibody capable of binding to alpha-synuclein can be one of the antibodies disclosed in Table 10. For example, antibodies for detecting alpha-synuclein are can include, but are not limited to: rabbit monoclonal antibody clones 7E2, and 3G2 against phosphorylated S129 alpha-synuclein. Additional monoclonal antibodies against phosphorylated S129 alpha-synuclein can include, for example, Abcam clones MJF-R13 (8-8), P/N ab168381 and P-syn/81A, P/N ab184674 or WAKO clone pSyn #64, P/N 015-25191. Mouse monoclonal antibody clone LB509 against alpha-synuclein regardless of S129 phosphorylation status can be used (for example, Abcam (P/N ab27766)). Anti-alpha-synuclein mouse monoclonal antibody clone 5C12 (S129 phosphorylation independent; ATCC® No. PTA-9197) and anti-phosphorylated S129-alpha-synuclein mouse monoclonal antibody clone 11A5 (ATCC® No. PTA-8222) from Prothena Corporation may also be used.

In some of the methods disclosed herein, the primary antibody capable of binding to the nerve feature is selected from an antibody capable of binding a protein selected from the group consisting of: ubiquitin C-terminal hydrolase L1 (PGP9.5, UCHL1, NDGOA; PARK5; PGP95; SPG79; Uch-L1; HEL-117; PGP 9.5; HEL-S-53), RNA binding fox-1 homolog 3 (RBFOX3, FOX3, NEUN, FOX-3, HRNBP3), microtubule associated protein 2 (MAP2, MAP2A, MAP2B, MAP2C), 160 kDa neurofilament medium (NEFM, NFM, NEF3, NF-M), 200 kDa neurofilament heavy (NEFH, NFH, CMT2CC), synaptophysin (SYP, MRX96, MRXSYP), and discs large MAGUK scaffold protein 4 (DLG4, DLGH4, PSD-95, PSD95, SAP-90, SAP90, SAP90A).

In some of the methods disclosed herein, the methods further comprise: contacting the section with a first secondary antibody having a first label conjugated thereto, wherein the first secondary antibody is immunoreactive with the primary antibody capable of binding phosphorylated alpha-synuclein, and contacting the section with a second secondary antibody having a second label conjugated thereto, wherein the second secondary antibody is immunoreactive with the primary antibody capable of binding the nerve feature.

In some of the methods disclosed herein, the methods further comprise: contacting the section with a set of reagents reactive with the first label of the first secondary antibody to generate a first detectable signal in proximity to phosphorylated alpha-synuclein in the sample; and contacting the section with a set of reagents reactive with the second label of the second secondary antibody to generate a second detectable signal in proximity to the nerve feature in the sample.

In some of the methods disclosed herein, the methods further comprise denaturing immunocomplexes in the sample after contacting the section with the primary antibody capable of binding phosphorylated alpha-synuclein and before contacting the section with the primary antibody capable of binding the nerve feature.

In some of the methods disclosed herein, the section is contacted with at least one protease before being contacted with a primary antibody. In certain methods, the method further comprises contacting the section with at least one phosphatase.

In some of the methods disclosed herein, the primary antibody capable of binding phosphorylated alpha-synuclein and the primary antibody capable of binding the nerve feature are from the same host species.

In some of the methods disclosed herein, the first detectable signal and the second detectable signal are different. In some of the methods disclosed herein, the first detectable signal is silver stain and the second detectable signal is QM-Dabsyl or Fast-Red.

Also provided herein are methods for diagnosing PD in a subject, said method comprising: (a) obtaining a biological sample comprising at least one nerve feature from a subject suspected of having PD; (b) detecting whether phosphorylated alpha-synuclein localizes within the nerve feature in the sample by contacting the sample with an anti-PGP.5 antibody and determining co-localization between phosphorylated alpha-synuclein and PGP9.5; and (c) diagnosing the subject with PD when it is determined in the affirmative that there is co-localization between phosphorylated alpha-synuclein and PGP9.5 in the nerve feature. In some instances, the method may be accomplished without the use of the anti-PGP9.5 antibody when nerve features can be identified without the signal associated with the anti-PGP9.5 antibody. For instance, an experienced pathologist may be able to recognize a nerve feature in a tissue sample without such signal. The pathologist can diagnose the subject with PD by presence of the signal associated with the phosphorylated alpha-synuclein localized with the nerve feature.

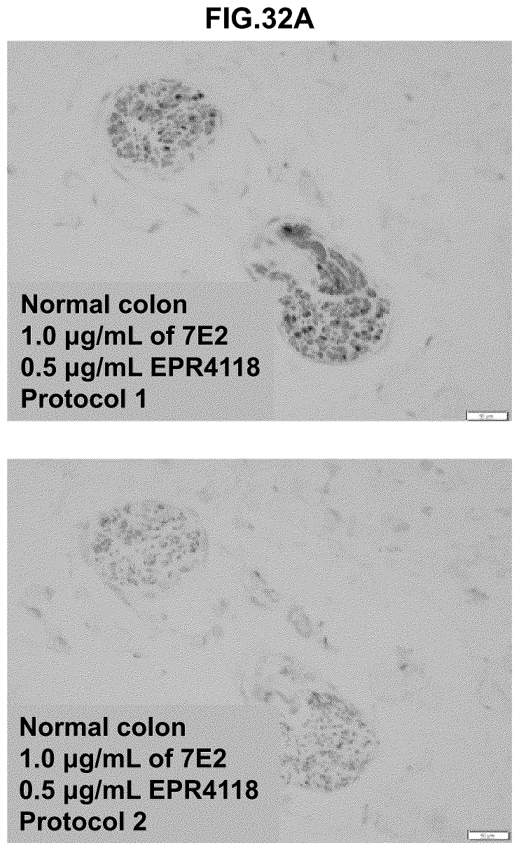

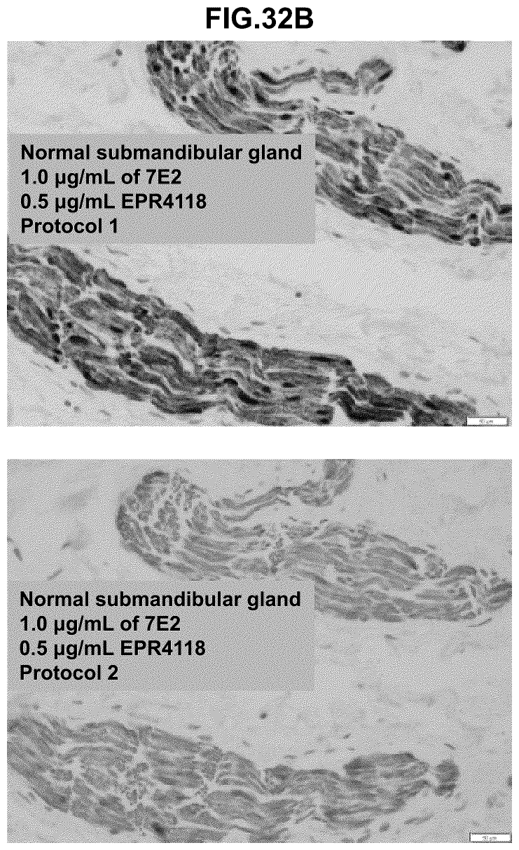

Some methods can include, for example, scoring phosphorylated alpha-synuclein and nerve features detected in the sample. In some methods, the number of nerve features in an entire slide are counted and expressed as percentage of nerve features containing phosphorylated alpha-synuclein. In certain methods where a nerve feature is identified by the presence of a target nerve feature protein, the presence of phosphorylated alpha-synuclein protein and the target nerve feature protein can include determining an absolute number of nerve features staining with the phosphorylated alpha-synuclein antibody in the sample using a 5×5 ocular grid with an area of 0.25 square millimeter, determining an absolute number of nerve features staining with the target nerve feature antibody in the sample using a 5×5 ocular grid with an area of 0.25 square millimeter, extrapolating the absolute number of cells staining with the phosphorylated alpha-synuclein antibody to a number of nerve features in a 1 square millimeter region, and extrapolating the absolute number of nerve features staining with the target nerve feature antibody to a number of nerve features in a 1 square millimeter region, thereby generating a score of phosphorylated alpha-synuclein protein and the target nerve feature protein. Thus, the method can include counting the number of nerve features staining positive for phosphorylated alpha-synuclein and the number of nerve features staining positive for the target nerve feature protein in the grid (such as phosphorylated alpha-synuclein and/or the target nerve feature positive nerve features). In one specific example, those values are inserted into the following formula: cell count/(0.0156×# of grids). This formula is dependent on the diameter of the objective which is variable between models even with equal magnification. Other formulas can be used if a different magnification or a different objective is used. In some examples, randomly selected regions of the sample are selected for scoring. Thus, as shown in FIG. 32, each co-localized black and yellow dot in the image represents nerve features staining with phosphorylated alpha-synuclein and the target nerve feature protein.

Provided herein are methods for determining whether a subject has Parkinson's Disease (PD), methods for diagnosing PD in a subject, and methods for diagnosing and treating PD in a subject by, in part, diagnosing a subject with PD when it is determined in the affirmative that there is co-localization between phosphorylated alpha-synuclein and a nerve feature in a biological sample from the subject. For example, if the sample obtained from the subject is analyzed or scored using the methods provided herein as having an increased co-localization of phosphorylated alpha-synuclein with nerve features relative to a normal sample (e.g., non-PD sample of the same tissue type), the subject can be determined to be, or diagnosed, as one having Parkinson's disease. For example, an increase of at least 20%, at least 40%, at least 50%, at least 75%, at least 80%, at least 90%, at least 100%, at least 200% or at least 500% (such as an increase of at least 2-fold, at least 5-fold, or at least 10-fold) in phosphorylated alpha-synuclein within nerve features in the tissue sample as compared to the phosphorylated alpha-synuclein within nerve features in a normal sample (such as a reference value or range of values for such a sample). In contrast, if the biological sample obtained from the subject is analyzed or scored using the methods provided herein as having a similar or decreased phosphorylated alpha-synuclein within nerve features relative to a normal sample (e.g., non-PD sample), the subject can be determined to be, or diagnosed as, one not-likely to have Parkinson's disease. In some examples, the disclosed methods can further include administering one or more therapies or treatments to the subject if the subject is determined to be, or diagnosed, as one having Parkinson's disease.

Also provided herein are methods of treating a subject diagnosed with PD, comprising administering to the subject an effective regime of an alpha-synuclein antibody, wherein an antibody capable of binding phosphorylated alpha-synuclein and an antibody capable of binding a nerve feature have been shown to co-localize in a nerve feature in a skin sample from the subject. In certain methods, a subject determined to have PD, can be treated by administering to the subject an effective regime of an alpha-synuclein antibody, wherein the subject was determined to have PD by any of the methods described herein.

As used herein, the terms “treat”, “treatment” and “treating” refer to the amelioration of one or more of the primary or secondary symptoms of Parkinson's disease and related movement disorders. Primary symptoms of Parkinson's disease include, but are not limited to tremor in an extremity while the extremity is at rest, generalized slowness of movement (bradykinesia), increased muscle rigidity or stiffness, gait or balance problems (postural dysfunction). Secondary symptoms of Parkinson's include, but are not limited to, difficulty initiating or resuming movements, loss of fine motor skills, lack of arm swing on the affected side of the body while walking, foot drag on the affected side of the body, decreased facial expression, voice and/or speech changes, cognitive disorders, sleep disorders, gastrointestinal dysfunction, and feelings of depression or anxiety.

Related movement disorder that may also be treated by the methods of the present disclosure include, but are not limited to, akathisia, akinesia (lack of movement), athetosis (contorted torsion or twisting), ataxia, ballismus (violent involuntary rapid and irregular movements), Cerebral palsy, choreas (e.g. Syndenham's chorea, rheumatic chorea, Huntington's disease), dystonias (e.g. dystonia musularum, belpharospasm, Writer's cramp, spasmodic torticollis), geniospasm (episodic involuntary up and down movements of the chin and lower lip) myoclonus, Restless Legs Syndrome (RLS), spasms, stereotypic movement disorder, stereotypy, Tardive dyskinesia, tic disorders (Tourette's syndrome, postural tremor, kinetic tremor, essential tremor, cerebellar tremor, physiological tremor), and Wilson's disease.

Prior to the initiation of treatment and/or therapy, all subjects should be evaluated and managed by a multidisciplinary team with expertise and experience in neurodegenerative disorders and Parkinson's disease. Subjects with PD typically have a medical professional and multidisciplinary health care team made up of doctors from different specialties including, but not limited to, neurologists, occupational therapists, physical therapists, counselors, social workers, registered dietitians, and speech therapists. After PD is diagnosed in a subject, a medical professional or team of medical professionals will typically recommend one or several treatment options including one or more of prescribed medications (for example, Benztropine mesylate (Cogentin), Entacapone (Comtan), Dopar, Larodopa, Levodopa and carbidopa (Sinemet), Pramipexole (Mirapex), Rasagiline (Azilect), Ropinirole Hcl (Requip), Rotigotine (Neupro), Safinamide (Xadago), Tasmar, or Trihexphenidyl (Artane)), surgery (for example, deep brain stimulation, pallidotomy, thalamotoy, or gamma knife), alternative therapies (Coenzyme Q10, massage, acupuncture, Tai Chi, yoga, Alexander technique, or meditation), and/or healthy eating and exercise. A treatment regimen is determined by a medical professional or team of medical professionals and can be specific to each subject. Those skilled in the art are familiar with various other treatments for PD.

In some such methods, the effective regime of an alpha-synuclein antibody comprises an alpha-synuclein antibody selected from the group consisting of: a monoclonal antibody binding within residues 1-20 of alpha-synuclein, 1-10 of alpha synuclein, 4-15 of alpha-synuclein, 91-99 of alpha-synuclein, 117-123 of alpha-synuclein, 118-126 of alpha-synuclein, prasinezumab (PRX002), a humanized antibody having the CDR's of antibody clone 1H7 (ATCC Accession No. PTA-8220), a humanized antibody having the CDR's of antibody clone 9E4 (ATCC Accession No. PTA-8221), the CDR's of antibody clone NI-202.21D11, and the CDR's of antibody clone NI-202.12F4, such as, for example, alpha-synuclein antibodies disclosed in U.S. Pat. Nos. 8,092,801, 8,609,820, 8,790,644, 8,940,276, 9,580,493, which are incorporated by reference herein in their entirety.

Some such antibodies comprise a VH CDR1 comprising residues 31-35 of SEQ ID NO:49, a VH CDR2 comprising residues 50-68 of SEQ ID NO:49, a VH CDR3 comprising residues 101-102 of SEQ ID NO:49, a VL CDR1 comprising residues 23-33 of SEQ ID NO:50, a VL CDR2 comprising residues 49-55 of SEQ ID NO:50, and a VL CDR3 comprising residues 88-98 of SEQ ID NO:50. Some such antibodies comprise a VH CDR1 comprising SEQ ID NO:51, a VH CDR2 comprising SEQ ID NO:52, a VH CDR3 comprising SEQ ID NO:53, a VL CDR1 comprising SEQ ID NO:54, a VL CDR2 comprising SEQ ID NO:55 and a VL CDR3 comprising SEQ ID NO:56. Some such antibodies comprise a heavy chain comprising SEQ ID NO:57 and a light chain comprising SEQ ID NO:58.

(SEQ ID NO: 9 of U.S. Pat No. 8,940,276)
SEQ ID NO: 49
EVQLVQSGGGLVEPGGSLRLSCAVSGFDFEKAWMSWVRQAPGQGLQWVARIKSTADGGTTSYAAP
VEGRFIISRDDSRNMLYLQMNSLKTEDTAVYYCTSAHWGQGTLVTVSS
(SEQ ID NO: 12 of U.S. Pat No. 8,940,276)
SEQ ID NO: 50
QSVLTQPPSVSVSPGQTARITCSGEALPMQFAHWYQQRPGKAPVIVVYKDSERPSGVPERFSGSS
SGTTATLTITGVQAEDEADYYCQSPDSTNTYEVFGGGTKLTVL
(SEQ ID NO: 16 of U.S. Pat No. 9,580,493; NI-202.21D11-VH CDR1)
SEQ ID NO: 51
NYAMH
(SEQ ID NO: 17 of U.S. Pat No. 9,580,493; NI-202.21D11-VH CDR2)
SEQ ID NO: 52
WINAGNGKRKYSQKFQD
(SEQ ID NO: 18 of U.S. Pat No. 9,580,493; NI-202.21D11-VH CDR3)
SEQ ID NO: 53
EEDHAGSGSYLSMDV
(SEQ ID NO: 23 of U.S. Pat No. 9,580,493; NI-202.21D11-VK CDR1)
SEQ ID NO: 54
KSSQNVLYSSNNKNYLA
(SEQ ID NO: 24 of U.S. Pat No. 9,580,493; NI-202.21D11-VK CDR2)
SEQ ID NO: 55
WASTRES
(SEQ ID NO: 25 of U.S. Pat No. 9,580,493; NI-202.21D11-VK CDR3)
SEQ ID NO: 56
QQYYSSPLT
(SEQ ID NO: 10 of U.S. Pat No. 8,609,820)
SEQ ID NO: 57
EVQLVESGGGLVQPGGSLRLSCAASGFTFSNYGMSWVRQAPGKGLEWVASISSGGGSTYYPDNVK
GRFTISRDDAKNSLYLQMNSLRAEDTAVYYCARGGAGIDYWGQGTLVTVSS
(SEQ ID NO: 5 of U.S. Pat No. 8,609,820)
SEQ ID NO: 58
DIQMTQSPSSLSASVGDRVTITCKSIQTLLYSSNQKNYLAWFQQKPGKAPKLLIYWASIRKSGVP
SRFSGSGSGTDFTLTISSLQPEDLATYYCQQYYSYPLTFGGGTKLEIK

In some such methods, the antibody is prasinezumab (PRX002). In some such methods, the antibody comprises three light CDRs designated SEQ ID NOs: 18-20 respectively and three heavy chain CDRs designated SEQ ID NOs: 22-24 respectively. In some such methods, the antibody comprises a light chain designated SEQ ID NO:17 and a heavy chain designated SEQ ID NO:21.

>10680L|prasinezumab|Humanized|LC|
SEQ ID NO: 17
DIQMTQSPSSLSASVGDRVTITCKSIQTLLYSSNQKNYLAWFQQKPGKAPKLLIYWASIRKSGVPSRF
SGSGSGTDFTLTISSLQPEDLATYYCQQYYSYPLTFGGGTKLEIKRTVAAPSVFIFPPSDEQLKSGTASV
VCLLNNFYPREAKVQWKVDNALQSGNSQESVTEQDSKDSTYSLSSTLTLSKADYEKHKVYACEVTHQGLS
SPVTKSFNRGEC
prasinezumab LC CDR1
SEQ ID NO: 18
KSIQTLLYSSNQKNYLA
prasinezumab LC CDR2
SEQ ID NO: 19
WASIRKS
prasinezumab LC CDR3
SEQ ID NO: 20
QQYYSYPLT
>10680H|prasinezumab|Humanized|HC|
SEQ ID NO: 21
EVQLVESGGGLVQPGGSLRLSCAASGFTFSNYGMSWVRQAPGKGLEWVASISSGGGSTYYPDNVKGRF
TISRDDAKNSLYLQMNSLRAEDTAVYYCARGGAGIDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPELLGGPSVFLFPPKPKDTLMISRTPEVTCVVVDVSHEDPEVKENWYV
DGVEVHNAKTKPREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSKLTVDKSRWQQ
GNVFSCSVMHEALHNHYTQKSLSLSPGK
prasinezumab HC CDR1
SEQ ID NO: 22
NYGMS
prasinezumab HC CDR1
SEQ ID NO: 23
SISSGGGSTYYPDNVKG
prasinezumab HC CDR1
SEQ ID NO: 24
GGAGIDY

In some such methods, the antibody is 9E4, as disclosed in U.S. Pat. No. 8,609,820, which is incorporated by reference herein in its entirety. The method provides an antibody comprising a humanized heavy chain comprising the three Kabat CDRs of SEQ ID NO:29 and a humanized light chain comprising the three CDRs of SEQ ID NO:25 provided that position L36 (Kabat numbering) is occupied by F or Y and/or position L83 (Kabat numbering) is occupied by L or F and/or position H73 (Kabat numbering) is occupied by D or N, and/or position H93 (Kabat numbering) is occupied by S or A. In some such antibodies, position L36 (Kabat numbering) is occupied by F and position H73 (Kabat numbering) is occupied by D, and position H93 (Kabat numbering) is occupied by S. In some such antibodies, position L36 is occupied by F. In some such antibodies, position L83 is occupied by L. In some such antibodies position H73 is occupied by D. In some such antibodies, position H93 is occupied by A. In some such antibodies, position L36 is occupied by F and position L83 is occupied by L. In some such antibodies, position L36 is occupied by F and position H73 is occupied by D. In some such antibodies, position L36 is occupied by F and position H93 is occupied by A. In some such antibodies, position L36 is occupied by F, position L83 is occupied by L and position H73 is occupied by D. In some such antibodies, position L36 is occupied by F, position L83 is occupied by L and position H93 is occupied by A. In some such antibodies, position L36 is occupied by F, position L83 is occupied by L, position 1173 is occupied by D and position H93 is occupied by A. In some such antibodies, residues at positions L36, L83, H73 and H93 (Kabat numbering) are occupied by F, and position H73 (Kabat numbering) is occupied by D, and position H93 (Kabat numbering) is occupied by A. In some such antibodies, position L36 (Kabat numbering) is occupied by F and position H93 (Kabat numbering) is occupied by S. In some such antibodies, position H73 (Kabat numbering) is occupied by D and position H93 (Kabat numbering) is occupied by S. In some such antibodies, position 1173 (Kabat numbering) is occupied by D and position 1193 (Kabat numbering) is occupied by A. In some such antibodies, position H93 (Kabat numbering) is occupied by S. In some such antibodies, position 1173 (Kabat numbering) is occupied by N. In some such antibodies, position L36 (Kabat numbering) is occupied by F, position L83 (Kabat numbering) is occupied by L, position H73 (Kabat numbering) is occupied by D, and position H93 (Kabat numbering) is occupied by S. In some such antibodies, position L36 (Kabat numbering) is occupied by F, position L83 (Kabat numbering) is occupied by L and position H93 (Kabat numbering) is occupied by S. In some such methods, the antibody comprises three light CDRs designated SEQ ID NOs:26-28 respectively and three heavy chain CDRs designated SEQ ID NOs:30-32 respectively. In some such methods, the antibody comprises a light chain variable region designated SEQ ID NO:25 and a heavy chain variable region designated SEQ ID NO:29.

9E4 LC Variable Region
SEQ ID NO: 25
DIQMTQSPSSLSASVGDRVTITCKSIQTLLYSSNQKNYLAWYQQKPGKAPKLLIYWASIRKSG
VPSRFSGSGSGTDFTLTISSLQPEDFATYYCQQYYSYPLTFGGGTKLEIK
9E4 LC CDR1
SEQ ID NO: 26
KSIQTLLYSSNQKNYLA
9E4 LC CDR2
SEQ ID NO: 27
WASIRKS
9E4 LC CDR3
SEQ ID NO: 28
QQYYSYPLT
9E4 HC Variable Region
SEQ ID NO: 29
EVQLVESGGGLVQPGGSLRLSCAASGFTFSNYGMSWVRQAPGKGLEWVASISSGGGSTYYPDN
VKGRFTISRDNAKNSLYLQMNSLRAEDTAVYYCARGGAGIDYWGQGTLVTVSS
9E4 HC CDR1
SEQ ID NO: 30
NYGMS
9E4 HC CDR2
SEQ ID NO: 31
SISSGGGSTYYPDNVKG
9E4 HC CDR3
SEQ ID NO: 32
GGAGIDY

In some such methods, the antibody is NI-202.21D11. In some such methods, the antibody comprises three light CDRs designated SEQ ID NOs:34-36 respectively and three heavy chain CDRs designated SEQ ID NOs: 38-40 respectively. In some such methods, the antibody comprises a light chain variable region designated SEQ ID NO:33 and a heavy chain variable region designated SEQ ID NO:37.

NI-202.21D11 LC variable region
SEQ ID NO: 33
DVVMTQSPDSLAVSLGERATINCKSSQNVLYSSNNKNYLAWYQQKPGHPPKLLIYWASTRESG
VPDRFSGSGSGTDFTLTITSLQTEDVAVYYCQQYYSSPLTFGGGTKVEIK
NI-202.21D11 LC CDR1
SEQ ID NO: 34
KSSQNVLYSSNNKNYLA
NI-202.21D11 LC CDR2
SEQ ID NO: 35
WASTRES
NI-202.21D11 LC CDR3
SEQ ID NO: 36
QQYYSSPLT
NI-202.21D11 HC variable region
SEQ ID NO: 37
EVQLVESGAEVKKPGASVKVSCKASGYTFTNYAMHWVRQAPGQRLEWMGWINAGNGKRKYSQK
FQDRVTINRDTSASTIYMELSSLGSEDTAVYYCAREEDHAGSGSYLSMDVWGQGTLVTVSS
NI-202.21D11 HC CDR1
SEQ ID NO: 38
NYAMH
NI-202.21D11 HC CDR2
SEQ ID NO: 39
WINAGNGKRKYSQKFQD
NI-202.21D11 HC CDR3
SEQ ID NO: 40
EEDHAGSGSYLSMDV

In some such methods, the antibody is NI-202.12F4. In some such methods, the antibody comprises light heavy CDRs designated SEQ ID NOs:42-44 respectively and three heavy chain CDRs designated SEQ ID NOs: 46-48 respectively. In some such methods, the antibody comprises a light chain variable region designated SEQ ID NO:41 and a heavy chain variable region designated SEQ ID NO:45.

NI-202.12F4 LC variable region
SEQ ID NO: 41
QSVLTQPPSVSVSPGQTARITCSGEALPMQFAHWYQQRPGKAPVIVVYKDSERPSGVPERFSG
SSSGTTATLTITGVQAEDEADYYCQSPDSTNTYEVFGGGTKLTVL
NI-202.12F4LC
CDR1
SEQ ID NO: 42
SGEALPMQFAH
NI-202.12F4LC
CDR2
SEQ ID NO: 43
KDSERPS
NI-202.12F4LC CDR3
SEQ ID NO: 44
QSPDSTNTYEV
NI-202.12F4
SEQ ID NO: 45
HC variable region
EVQLVQSGGGLVEPGGSLRLSCAVSGFDFEKAWMSWVRQAPGQGLQWVARIKSTADGGTTSYA
APVEGRFIISRDDSRNMLYLQMNSLKTEDTAVYYCTSAHWGQGTLVTVSS
NI-202.12F4HC
CDR1
SEQ ID NO: 46
KAWMS
NI-202.12F4HC
CDR2
SEQ ID NO: 47
RIKSTADGGTTSYAAPVEG
NI-202.12F4HC
CDR3
SEQ ID NO: 48
TSAH

Also provided herein are methods for detecting phosphorylated alpha-synuclein, the method comprising: contacting a biological sample with a primary antibody capable of binding phosphorylated alpha-synuclein, and detecting the primary antibody capable of binding phosphorylated alpha-synuclein. In some of the methods, the sample is contacted with at least one protease before being contacted with the primary antibody capable of binding phosphorylated alpha-synuclein. In some methods, the method further comprises contacting the sample with at least one phosphatase. In some methods, the detecting comprising histochemical analysis. In some methods, the primary antibody capable of binding phosphorylated alpha-synuclein detects alpha-synuclein phosphorylated at residue S129. In certain methods, the primary antibody capable of binding phosphorylated alpha-synuclein is the 7E2 antibody clone or the 3G2 antibody clone. In some methods, the sample is fixed. In certain methods, the sample is a formalin fixed, paraffin embedded (FFPE) sample. In some methods, the sample is a frozen sample

In some methods, the method further comprises contacting the sample with a first secondary antibody having a first label conjugated thereto, wherein the first secondary antibody is immunoreactive with the primary antibody capable of binding phosphorylated alpha-synuclein. In some methods, the method comprises contacting the sample with a set of reagents reactive with the first label of the first secondary antibody to generate a first detectable signal in proximity to phosphorylated alpha-synuclein in the sample.

### Examples

Parkinson's disease is a progressive neurodegenerative disease characterized by the presence of Lewy bodies and Lewy neurites in the brain of subjects suffering from the disease. Lewy bodies are highly enriched with the aggregated form of alpha-synuclein (aSyn), a protein with poorly characterized functions. Aggregation of aSyn was also found in peripheral nerves from subject with Parkinson's disease. Aggregated aSyn can be detected using immunohistochemistry (IHC) and can be distinguished from non-aggregated aSyn by virtue of its resistance to protease treatment. However, prolonged protease treatment could degrade tissue morphology in IHC assays, leading to the need for an alternative method to differentiate aggregated aSyn from non-aggregated aSyn. An unusually high proportion of aggregated aSyn is found to be phosphorylated at Ser129 residue (pS129) compared with non-aggregated aSyn. As shown herein, a highly sensitive and specific assay for aggregated aSyn using pS129-aSyn as a surrogate marker for aSyn aggregation was developed.

### Materials

Tissue source. Scalp, abdomen area skin, colon, and submandibular gland samples were obtained from Banner Sun Health Research Institute (BSHRI) courtesy of Dr. Thomas G. Beach, director of Brain and Body Donation Program. In addition to standardized clinical assessments diagnosis of Parkinson's disease was confirmed in the tissue donors by the presence of Lewy bodies. Similarly the normal, non-PD control subjects' status was determined by lack of Lewy bodies. The samples (16 males, 8 females; ages 65-95; 15 PD, 9 non-PD) were obtained post-mortem and fixed in formalin. The formalin-fixed paraffin-embedded (FFPE) cortical brain blocks from normal individuals and subjects with PD were obtained from Roche Tissue Diagnostics (RTD) internal tissue bank and Folio Biosciences, respectively. A small cohort of FFPE skin biopsy blocks was obtained from 8 individuals (3 males, 5 females) 1 to 16 years of age through Folio Biosciences to assess the degree of pS129-aSyn staining that does not resemble aggregated aSyn. A much larger cohort of 72 FFPE skin biopsy blocks from 36 subjects were obtained from Dr. Ron Postuma at Montreal General Hospital Department of Neurology. Fifteen of the 36 subjects were diagnosed with PD and five with atypical Parkinsonism based on clinical examination of subjects' presented symptoms. The rest (16 subjects) were control subjects without PD. Two skin punch biopsies were performed on the subjects during clinical visits and FFPE blocks were transferred to Ventana Medical Systems through Prothena. All other tissue specimens were obtained from RTD internal tissue bank. Unless otherwise noted, thickness of all FFPE tissue sections was 4 μm.

Antibodies against alpha-synuclein (aSyn) and PGP9.5. Rabbit monoclonal antibody clones 7E2, and 3G2 against phospho-S129 aSyn were generated by Roche Diagnostics GmbH CPS R&D Early Development & Reagent Design (DXREAA) with a phospho-peptide corresponding to human aSyn residues 122-135 as immunogen. Additional monoclonal antibodies against phospho-S129 aSyn were purchased from Abcam (clones MJF-R13 (8-8), P/N ab168381 and P-syn/81A, P/N ab184674) or WAKO (clone pSyn #64, P/N 015-25191). Mouse monoclonal antibody clone LB509 against aSyn regardless of S129 phosphorylation status was purchased from Abcam (P/N ab27766). Anti-aSyn mouse monoclonal antibody clone 5C12 (S129 phosphorylation independent) and anti-phosphorylated S129 alpha-synuclein mouse monoclonal antibody clone 11A5 were gifts from Prothena Corp. Also see Table 10.

Three anti-human PGP9.5 antibodies were used in the present study. Two monoclonal antibodies, one rabbit (clone EPR4118, P/N ab108986) and the other mouse (clone 13C/I3C4, P/N ab8189), were purchased from Abcam. A rabbit polyclonal antibody with RTD P/N 760-4434 was obtained from Cell Marque™.

Enzymes. VENTANA® Protease 1 (P/N 760-2018) was used to remove non-aggregated aSyn in automated protease-resistant alpha-synuclein DAB IHC assay developed by pRED/Prothena. In one specific implementation of automated pS129-aSyn and PGP9.5 silver/yellow dual IHC assay (Bumblebee Assay Protocol 2 described below), recombinant bovine alkaline phosphatase (Roche, at 30 μg/mL in a stabilizing diluent (Roche P/N 06002919001)) and VENTANA® Protease 3 (P/N 760-2020) were used, respectively, to remove pS129-aSyn staining that did not resemble aggregated aSyn and enhance detection of pS129-aSyn.

Commercially available and user-provided reagents for automated phospho-S129-aSyn and PGP9.5 silver/yellow dual IHC assay and protease-resistant alpha-synuclein DAB IHC assay. Table 1 below contains the list of reagents including bulk solutions, enzymes, antibodies, detection kits, and ancillaries used in Bumblebee Assay Protocols 1 and 2 and DAB-based pRED/Prothena Assay Protocol for detection of protease-resistant aSyn (see Methods below).

TABLE 1
List of potential reagents used

| | | Retire number |
|---|---|---|
| | RTD/Ventana ™ | or overlabel |
| Name | Part # | used in protocols |
| EZ Prep (10x) | 950-102 | 05279771001 |
| ULTRA LCS | 650-210 | 05424534001 |
| SSC (10X) | 950-110 | 05353947001 |
| Reaction Buffer | 950-300 | 05353955001 |
| ULTRA CC1 | 950-224 | 05424569001 |
| ULTRA CC2 | 950-223 | 05424542001 |
| SilverWash II | 780-003 | 05446724001 |
| Hematoxylin II | 790-2208 | 05277965001 |
| Bluing Reagent | 760-2037 | 05266769001 |
| Protease I | 760-2018 | 05266688001 |
| ISH Protease 3 or Protease 3 | 780-4149/760-2020 | 5266718001/5266718001 |
| 7E2 Ab 1 μg/mL in DISCOVERY Goat Ig Block | N/A | ANTIBODY 2 |
| 5C12 Ab 1 μg/mL in 95028 | N/A | PREP KIT 9 |
| DISCOVERY Goat Ig Block | 760-6008 | Option 1 |
| pH adjust (500 mM Tris pH 10) | N/A | PRETREATMENT 4 |
| 30 μg/mL recombinant bovine AP | N/A | PRETREATMENT 5 |
| (Roche)diluted in Roche diluent with P/N 06002919001. | | |
| ultraView SISH DNP Detection Kit | 760-098 | 5572037001 |
| OptiView DAB Detection Kit | 760-700 | 6396500001 |
| pH adjust (500 mM Tris pH 10) | N/A | ANTIBODY 11 |
| QM-Peg8-Dabsyl 480 μM in 25% DMSO/75% QM Buffer | N/A | ANTIBODY 12 |
| (10 mM glycine pH 2/1.05M MgCl₂/0.05% Brij-35) | | |
| DISCOVERY UltraMap GaR-AP | 760-4314 | ANTIBODY 10 |
| EPR4118 PGP9.5 Ab 0.5 μg/mL in DISCOVERY Goat Ig Block | N/A | ANTIBODY 5 |
| ISH Peroxidase Inhibitor | 780-5061 | 07729014001 |

Equipment. BenchMark ULTRA instruments with the following serial numbers were used: 310520, 310841, 310934, 310940, 311000, 311112, 311276, 311279, and 311311.

### Methods

DAB staining of FFPE brain and skin sections from donors with and without Parkinson's disease using antibodies against aSyn or PGP9.5

FFPE brain sections were stained using VENTANA® ultraView Universal DAB Detection Kit or OptiView DAB IHC Detection Kit in an automated VENTANA® BenchMark ULTRA instrument with standard complement of bulk solutions. Following deparaffinization, slides were subjected to one of three antigen retrieval procedures singly or in combination or none at all as indicated. The three antigen retrieval procedures were: 1) VENTANA® Protease 1 (P/N 760-2018) diluted approximately 3.7-fold on slide with Reaction Buffer and incubated for 4 minutes at 36° C., 2) VENTANA® ULTRA CC1 solution (P/N 950-224) for either 32 minutes (for antibodies against alpha-synuclein) or 64 minutes (for antibodies against PGP9.5) at 100° C. unless otherwise noted, and 3) alkaline phosphatase (AP, recombinant from Pichia pastoris, highly active EIA grade, Roche) for 2 hours at 36° C. Alkaline phosphatase was cocktailed in a dispenser at 50 μg/mL in a stabilizing solution (Roche P/N 06002919001) and 1 drop was applied to tissue slide immersed in VENTANA® Reaction Buffer following sequential applications of 1 drop of a 1:1 mixture of 0.5M Tris pH 10 and Reaction Buffer and 1 drop of 20 mM MgCl2. Primary antibodies were either hand applied or automatically applied from dispensers at indicated concentrations. Incubations for all primary antibodies were performed at 36° C. for 32 minutes unless otherwise noted. When detection was performed using VENTANA® ultraView Universal DAB Detection Kit (P/N 760-500) or OptiView DAB IHC Detection Kit (P/N 760-700), the ultraView Universal HRP Multimer, the OptiView HQ Universal Linker, and the OptiView HRP Multimer were incubated at 36° C. for 16 minutes unless otherwise noted and DAB stain was deposited using standard conditions in U ultraView DAB or U OptiView DAB IHC procedures. Counterstaining was performed on instrument using VENTANA® Hematoxylin II (P/N 790-2208, 4 minutes at 36° C.) and Bluing Reagent (P/N 760-2037, 4 minutes at 36° C.). Slides were coverslipped in xylene following alcohol dehydration in a Tissue-Tek automated slide stainer & coverslipper (Sakura).

Automated phospho-S129-alpha-synuclein and PGP9.5 silver/yellow dual IHC assay (Bumblebee Assay Protocols). FFPE skin sections were placed in a VENTANA® BenchMark ULTRA instrument with standard complement of bulk solutions including ultraView Silver Wash II (Ventana P/N 780-003). Two protocols (Protocol 1 and Protocol 2) based on the validated U Triplex IHC Silver_QM_DAB procedure were used to stain consecutive skin sections. Following deparaffinization, Bumblebee Assay Protocol 1 (see Table 2 below) had no antigen retrieval steps prior to incubation with 7E2 anti-phosphorylated S129 alpha-synuclein antibody. In contrast, alkaline phosphatase (Roche, diluted to 30 μg/mL in a stabilizing solution (Roche P/N 06002919001)) and VENTANA® Protease 3 (P/N 760-2020) were applied sequentially (with washing in between the treatments) to the slides prior to 7E2 antibody incubation in Bumblebee Assay Protocol 2 (see Table 3 below). Conditions for alkaline phosphatase and protease treatments are 8 minutes at 37° C. and 4 minutes at 36° C., respectively. Both Bumblebee Assay Protocols 1 and 2 had the same antibody incubation and detection steps: 1) incubation with 7E2 antibody for 32 minutes at 36° C., 2) detection of phospho-aSyn with VENTANA® ultraView SISH DNP kit using HRP-conjugated goat anti-rabbit antibody (incubation at 36° C. for 48 minutes) and precipitation of metallic silver, 3) deactivation of primary and secondary antibody complexes with VENTANA® ULTRA CC2 bulk solution (P/N 950-223) at 100° C. for 16 minutes which also serves as antigen retrieval for PGP9.5, 4) incubation with EPR4118 anti-PGP9.5 antibody at 36° C. for 32 minutes, 5) incubation with AP-conjugated goat anti-rabbit antibody (VENTANA® DISCOVERY UltraMap anti-Rb Alk Phos, P/N 760-4314) for 32 minutes at 36° C., and 6) detection of PGP9.5 with AP-induced activation of phosphate-protected quinone methide conjugated to 4-(diethylamino)azobenene-4-sulfonamide (DABSYL). Approximate on-slide concentrations of 7E2 and ERP4118 antibodies were, respectively, 0.27 and 0.14 μg/mL after dilution with previously applied 1× Reaction Buffer (Ventana P/N 950-300). Counterstaining was performed on instrument using VENTANA® Hematoxylin II (P/N 790-2208, 8 minutes at 36° C.) and Bluing Reagent (P/N 760-2037, 8 minutes at 36° C.). Slides were coverslipped in xylene following alcohol dehydration in a Tissue-Tek automated slide stainer & coverslipper (Sakura). See Table 2 for a summary of Protocol 1 and Table 3 for a summary of Protocol 2.

TABLE 2
Summary of Protocol 1

| Step # | Description |
|---|---|
| 1 | Deparaffinization [selected] |
| 2 | Warmup slide to [72° C.] from medium temperatures |
| | (deparaffinization) |
| 3 | Pre-primary peroxidase inhibit. [selected] |
| 4 | Inhibitor [selected] |
| 5 | Primary Antibody [selected] |
| 6 | Single reagent application [selected] |
| 7 | Apply one drop of [ANTIBODY 2] (antibody), apply |
| | coverslip, and incubate for [32 minutes] |
| 8 | Silver detection [selected] |
| 9 | Detection ultraVIEW SISH [selected] |
| 10 | Apply one drop of SIL, ISH, DNP, HRP, apply coverslip, |
| | and incubate for [48 minutes] |
| 11 | Apply one drop of SIL, ISH, DNP, CHRC, and incubate |
| | for [12 minutes] |
| 12 | 2^{nd} detection [selected] |
| 13 | Antibody denaturation [selected] |
| 14 | Warmup slide to [100° C.] from all temperatures (antibody) |
| 15 | Incubate for [16 minutes] (antibody) |
| 16 | Primary antibody (2) [selected] |
| 17 | Single reagent application (2) [selected] |
| 18 | Apply one drop [ANTIBODY 5] (Antibody 11), apply coverslip, |
| | and incubate for [32 minutes] |
| 19 | Secondary Antibody (2) [selected] |
| 20 | Single application (2) [selected] |
| 21 | Blocker (2) [selected] |
| 22 | Apply one drop of [OPTION 1] (Option 2), apply coverslip, |
| | and incubate for 4 minutes |
| 23 | Apply one drop of [ANTIBODY 10], (Antibody 12), and |
| | incubate for [32 minutes] |
| 24 | Chromogen block (2) [selected] |
| 25 | Apply two drops [ANTIBODY 11] (Antibody 15), apply |
| | coverslip, and incubate for 4 minutes |
| 26 | Apply one drop of [ANTIBODY 12] (Antibody 16), and |
| | incubate for [32 minutes] |
| 27 | Counterstain [selected] |
| 28 | Apply one drop of [HEMATOXYLIN II] (Counterstain), |
| | apply coverslip, and incubate for [8 minutes] |
| 29 | Post counterstain [selected] |
| 30 | Apply one drop of [BLUING REAGENT] (Post-counterstain), |
| | apply counterstain, and incubate for [8 minutes] |

TABLE 3
Summary of Protocol 2

| Step # | Description |
|---|---|
| 1 | Deparaffinization [selected] |
| 2 | Warmup slide to [72° C.] from medium temperatures |
| | (deparaffinization) |
| 3 | Pretreatment [selected] |
| 4 | Warmup slide to [37° C.] from 37-60° C. temperatures |
| | (Pretreatment #1 Temp RB) |
| 5 | Research Fork #36 [selected] |
| 6 | Apply one drop of [PRETREATMENT 4] (Pretreatment #2), |
| | apply coverslip, and incubate for 4 minutes |
| 7 | Apply one drop of [PRETREATMENT 5] (Pretreatment #3), |
| | and incubate for [8 minutes] |
| 8 | Post cell conditioning enzyme [selected] |
| 9 | Apply one drop of [PROTEASE 3] (Enzyme), apply coverslip, |
| | and incubate for [4 minutes] |
| 10 | Pre-primary peroxidase inhibit. [selected] |
| 11 | Inhibitor [selected] |
| 12 | Primary Antibody [selected] |
| 13 | Single reagent application [selected] |
| 14 | Apply one drop of [ANTIBODY 2] (antibody), apply coverslip, |
| | and incubate for [32 minutes] |
| 15 | Silver detection [ selected] |
| 16 | Detection ultraVIEW SISH [selected] |
| 17 | Apply one drop of SIL, ISH, DNP, HRP, apply coverslip, |
| | and incubate for [48 minutes] |
| 18 | Apply one drop of SIL, ISH, DNP. CHRC, and incubate |
| | for [12 minutes] |
| 19 | 2^{nd} detection [selected] |
| 20 | Antibody denaturation [selected] |
| 21 | Warmup slide to [100° C.] from all temperatures (antibody) |
| 22 | Incubate for [16 minutes] (antibody) |
| 23 | Primary antibody (2) [selected] |
| 24 | Single reagent application (2) [selected] |
| 25 | Apply one drop [ANTIBODY 5] (Antibody 11), apply |
| | coverslip, and incubate for [32 minutes] |
| 26 | Secondary Antibody (2) [selected] |
| 27 | Single application (2) [selected] |
| 28 | Blocker (2) [selected] |
| 29 | Apply one drop of [OPTION 1] (Option 2), apply |
| | coverslip, and incubate for 4 minutes |
| 30 | Apply one drop of [ANTIBODY 10], (Antibody 12), |
| | and incubate for [32 minutes] |
| 31 | Chromogen block (2) [selected] |
| 32 | Apply two drops [ANTIBODY 11] (Antibody 15), apply |
| | coverslip, and incubate for 4 minutes |
| 33 | Apply one drop of [ANTIBODY 12] (Antibody 16), and |
| | incubate for [32 minutes] |
| 34 | Counterstain [selected] |
| 35 | Apply one drop of [HEMATOXYLIN II] (Counterstain), |
| | apply coverslip, and incubate for [8 minutes] |
| 36 | Post counterstain [selected] |
| 37 | Apply one drop of [BLUING REAGENT] (Post-counterstain), |
| | apply counterstain, and incubate for [8minutes] |

Automated protease-resistant alpha-synuclein DAB IHC assay (pRED/Prothena Assay Protocol). As aggregated alpha-synuclein is known to be a poor substrate for proteases, histochemical staining of aggregated aSyn following removal of non-aggregated alpha-synuclein by protease can be employed for detection of Lewy bodies. An assay based on this principle had been developed by Roche pRED in collaboration with Prothena Biosciences to detect aggregated alpha-synuclein in skin biopsy (pRED/Prothena Assay Protocol). This assay relies on the 5C12 antibody whose epitope has been mapped to alpha-synuclein residues 109 to 120, a region outside of the Ser129 phosphorylation site. The pRED/Prothena Protocol is shown in Table 4, and the protocol employed in this study is shown as Table 5. For the pRED/Prothena Assay Protocol, protease treatment was performed for 12 minutes at 36° C. using VENTANA® Protease 1 (P/N 760-2018), and 5C12 antibody was incubated for 20 minutes at ambient temperature (instrument slide heater disabled) with approximate on-slide concentration of 0.27 μg/mL after dilution with previously applied 1× VENTANA® Reaction Buffer (P/N 950-300). As shown in Table 4, dispenser formulation of 5C12 antibody is 1 μg/mL diluted in VENTANA® Antibody Diluent (P/N 251-018, Antibody Diluent without Brij-35, internal P/N 95028). Following incubation with primary antibody, aggregated alpha-synuclein was detected using VENTANA® OptiView DAB IHC Detection Kit (P/N 760-700). Counterstaining was performed on instrument using VENTANA® Hematoxylin II (P/N 790-2208, 8 minutes at 36° C.) and Bluing Reagent (P/N 760-2037, 8 minutes at 36° C.). Slides were coverslipped in xylene following alcohol dehydration in a Tissue-Tek automated slide stainer & coverslipper (Sakura).

TABLE 4
Summary of a protocol for protease-resistant alpha-synuclein

| Step # | Description |
|---|---|
| 1 | Paraffin [selected] |
| 2 | Deparaffinization [selected] |
| 3 | Warmup slide to [75° C.] from medium temperatures |
| | (deparaffinization) |
| 4 | Pre cell conditioning enzyme [selected] |
| 5 | Apply one drop of Protease 1 (Catalog No. 760-2018), |
| | apply coverslip, and incubate for 12 minutes |
| 6 | Pre-primary peroxidase inhibitor [selected] |
| 7 | Apply coverslip, one drop of Primary Antibody |
| | (without heating): alpha-Synuclein 5C12 (1 μg/mL) |
| | clone for 20 minutes |
| 8 | Counterstain [selected] |
| 9 | Apply one drop of [HEMATOXYLIN II] (Counterstain), |
| | apply coverslip, and incubate for 8 minutes |
| 10 | Post counterstain [selected] |
| 11 | Apply one drop of Bluing Reagent (Post-counterstain), |
| | apply counterstain, and incubate for 8 minutes |

TABLE 5
Summary of a protocol for protease-resistant alpha-synuclein

| Step # | Description |
|---|---|
| 1 | Paraffin [selected] |
| 2 | Deparaffinization [selected ] |
| 3 | Warmup slide to [75° C.] from medium temperatures |
| | (deparaffinization) |
| 4 | Pre cell conditioning enzyme [selected] |
| 5 | Apply one drop of [PROTEASE 1] (Enzyme), apply |
| | coverslip, and incubate for [12 minutes] |
| 6 | Pre-primary peroxidase inhibitor [selected] |
| 7 | Primary Antibody [selected] |
| 8 | Primary Antibody temperature [selected] |
| 9 | Primary Antibody no heat [selected] |
| 10 | Apply coverslip, one drop of [PREP KIT 9] (Antibody), and |
| | incubate for [12 minutes] |
| 11 | Counterstain [selected] |
| 12 | Apply one drop of [HEMATOXYLIN II] (Counterstain), |
| | apply coverslip, and incubate for [8 minutes] |
| 13 | Post counterstain [selected] |
| 14 | Apply one drop of [BLUING REAGENT] (Post-counterstain), |
| | apply counterstain, and incubate for [8 minutes] |

Slide evaluation and scoring of aSyn signals. Slides were scored by a board-certified pathologist or assay development scientists with extensive experience evaluating aSyn staining patterns in skin. For pS129-aSyn and PGP9.5 silver/yellow dual IHC assay, number of total nerve features with yellow PGP9.5 stain was recorded along with number of nerve features exhibiting discrete and/or diffuse granular silver pS129-aSyn stain. In a typical skin section, yellow PGP9.5 stain was observed in nerve bundles and arrector pili muscle and also surrounding eccrine/sebaceous glands and blood vessels. At least 25 nerve features were counted per slide, but often more were counted to ensure assay sensitivity was not adversely affected by low numbers of nerve features evaluated. In slides with limited skin area and total number of nerve features available for evaluation were less than 25, the entire skin area was scanned and total number of nerve features counted. Results were expressed as percentages of nerve features with a particular type of pS129-aSyn stain. Slides stained using protease-resistant aSyn DAB IHC assay were scored similarly except nerve features were recognized by morphology. In cases where tissue morphology was significantly affected by treatment with Protease 1 the numbers of total nerve features were derived from neighboring slides stained using pS129-aSyn and PGP9.5 silver/yellow dual IHC assay.

Statistical Analyses. Association between presence of phosphorylated alpha-synuclein signal and subjects' clinical statuses was evaluated by chi-square test using Minitab 17 statistical software.

### Example 1. Characterization of 7E2 Rabbit Monoclonal Antibody Against pS129-aSyn

The anti-phosphorylated S129 alpha-synuclein rabbit monoclonal antibody 7E2 was generated by Roche Diagnostics GmbH CPS R&D Early Development & Reagent Design. The immunogen used for the generation of 7E2 is a peptide conjugated to KLH via an N-terminal Cys residue. The sequence of this peptide, NEAYEMPpSEEGYQD (SEQ ID NO:59), corresponds to residue 122-135 of human alpha-synuclein. Surface plasmon resonance (Biacore) experiments showed 7E2 antibody to have high specificity toward the alpha-synuclein (122-135, pS129) immunogen peptide phosphorylated at Ser129 compared with the same peptide not phosphorylated at Ser129. The 7E2 antibody exhibited fast association rate and slow linear dissociation rate toward alpha-synuclein (122-135, pS129) peptide.

Specificity of 7E2 anti-phosphorylated S129 alpha-synuclein antibody in immnohistochemical context was examined using a human tissue microarray containing 30 cores of normal and 29 cores of cancer tissue from various anatomical sites (SuperBioChips Laboratories, Seoul, Korea, P/N BC8). The Tour of Body/Tour of Tumor (ToB/ToT) analysis indicated that the only non-neuronal staining observed with 7E2 antibody was macrophages (expression of alpha-synuclein in macrophages has been reported previously). See Tables 6-9 for summary of ToB/ToT analyses. The ToB/ToT results demonstrate that the protocols are both accurate and specific.

TABLE 6
Summary of Tour of Body analysis for Protocol 1

| | | | | PGP9.5 | | | |
|---|---|---|---|---|---|---|---|
| | | | | Percent | PGP9.5 | aSyn | |
| | | | | Positive | Average | Staining | |
| No. | Age | Sex | Tissue Type | Cells | Intensity | present | Comments |
| 1 | 63 | F | Skin | 2 | 2.5 | No | NA |
| 2 | 44 | F | Breast | 1 | 2 | No | Silver dusting around blood vessel |
| 3 | 53 | F | Spleen | 1 | 2.5 | No | NA |
| 4 | 51 | M | Lymph node | 5 | 2.5 | Yes | PGP9.4: blood vessels 2.5+/lymphocytes 1+; |
| | | | | | | | Asyn: macrophages |
| 5 | 57 | M | Skeletal muscle | 1 | 2.5 | No | Around blood vessels |
| 6 | 39 | M | Lung | <1 | 2.5 | Yes | PGP9.5: rare stromal cell; A Syn: cytoplasm of |
| | | | | | | | macrophages |
| 7 | 69 | M | Salivary gland, | 10 | 2.5 | No | around glands |
| | | | sublingual | | | | |
| 8 | 70 | M | Liver | 3 | 2.75 | No | Air under coverslip? |
| 9 | 65 | M | Gallbladder | 2 | 2.75 | No | Silver dusting in nerve and around blood vessel |
| 10 | 73 | F | Pancreas | 10 | 3 | No | PGP9.5 in islet cells; air under coverslip? |
| 11 | 24 | M | Tonsil | 10 | 1 | No | Silver dusting around blood vessels |
| 12 | 69 | M | Esophagus | 10 | 2.75 | No | PGP9.5: around blood vessels |
| 13 | 31 | M | Stomach, antrum | 5 | 2.75 | No | PGP9.5: around blood vessels and in nerve |
| 14 | 59 | M | Stomach, fundus | 5 | 2.75 | No | PGP9.5: around blood vessels and in nerve |
| 15 | 66 | F | Small bowel | 20 | 2.75 | No | PGP9.5: around blood vessels and in nerve and |
| | | | | | | | ganglion cells |
| 16 | 73 | M | Colon | 15 | 2.75 | No | PGP9.5: around blood vessels and in nerve and |
| | | | | | | | ganglion cells |
| 17 | 67 | M | Rectum | 5 | 2.75 | No | PGP9.5: around blood vessels and in nerve and |
| | | | | | | | ganglion cells |
| 18 | 48 | M | Kidney, cortex | 20 | 3 | No | PGP9.5: in tubules |
| 19 | 48 | M | Kidney, medulla | 25 | 3 | No | PGP9.5: in tubules and in nerve |
| 20 | 32 | M | Urinary bladder | 3 | 2.75 | No | NA |
| 21 | 32 | M | Prostate | 2 | 2.5 | No | Around blood vessels |
| 22 | 70 | M | Testis | 20 | 2.75 | No | PGP9.5: stains germ cells |
| 23 | 42 | F | Uterine cervix | 3 | 2.75 | No | NA |
| 24 | 39 | F | Endometrium | 2 | 1.5 | No | Around blood vessels |
| 25 | 42 | F | Myometrium | <1 | 1.5 | No | NA |
| 26 | 34 | F | Placenta | 20 | 2.75 | No | Stains trophoblast cells |
| 27 | 42 | M | Adrenal gland | 15 | 2.75 | No | Around blood vessels |
| 28 | 39 | M | Thyroid | 2 | 2.5 | No | Around blood vessels |
| 29 | 59 | M | Cerebrum | 95 | 2.75 | No | NA |
| 30 | 1 | M | Cerebellum | 95 | 2.75 | No | NA |

TABLE 7
Summary of Tour of Body Analysis for Protocol 2

| | | | | PGP9.5 | | | |
|---|---|---|---|---|---|---|---|
| | | | | Percent | PGP9.5 | aSyn | |
| | | | | Positive | Average | Staining | |
| No | Age | Sex | Tissue type | Cells | Intensity | present | Comments |
| 1 | 63 | F | Skin | 3 | 2.5 | No | stromal cells and nerve staining |
| 2 | 44 | F | Breast | <1 | 2 | No | NA |
| 3 | 53 | F | Spleen | 2 | 2.5 | No | Around blood vessels |
| 4 | 51 | M | Lymph node | 5 | 2 | Yes | PGP9.5: Blood vessels 2+/lymphocytes 1+; |
| | | | | | | | Asyn: macrophages |
| 5 | 57 | M | Skeletal muscle | 1 | 2.5 | No | Around blood vessels |
| 6 | 39 | M | Lung | 0 | 0 | Yes | Asyn in macrophages |
| 7 | 69 | M | Salivary gland, | 10 | 2.5 | No | Around glands, some tissue tearing/folding |
| | | | sublingual | | | | |
| 8 | 70 | M | Liver | 5 | 2.75 | No | NA |
| 9 | 65 | M | Gallbladder | 3 | 2.75 | No | NA |
| 10 | 73 | F | Pancreas | 10 | 3 | No | Islet cell staining |
| 11 | 24 | M | Tonsil | 10 | 1.5 | No | Germinal center staining |
| 12 | 69 | M | Esophagus | 10 | 2.5 | No | Around blood vessels |
| 13 | 31 | M | Stomach, antrum | 10 | 2.75 | No | Nerve and blood vessels |
| 14 | 59 | M | Stomach, fundus | 15 | 2.75 | No | Nerve and blood vessels |
| 15 | 66 | F | Small bowel | 20 | 2.75 | No | Nerve and blood, vessels |
| 16 | 73 | M | Colon | 15 | 2.75 | No | Nerve and blood vessels |
| 17 | 67 | M | Rectum | 15 | 2.75 | No | Nerve, ganglion cells and blood vessels |
| 18 | 48 | M | Kidney, cortex | 20 | 3 | No | tubules |
| 19 | 48 | M | Kidney, medulla | 25 | 3 | No | tubules |
| 20 | 32 | M | Urinary bladder | 3 | 2.75 | No | Nerve and blood vessels |
| 21 | 32 | M | Prostate | 10 | 2.5 | No | NA |
| 22 | 70 | M | Testis | 20 | 2.75 | No | Germ cells |
| 23 | 42 | F | Uterine cervix | 5 | 2 | No | NA |
| 24 | 39 | F | Endometrium | 0 | 0 | No | NA |
| 25 | 42 | F | Myometrium | 3 | 2 | No | NA |
| 26 | 34 | F | Placenta | 2 | 2.5 | No | trophoblast cells |
| 27 | 42 | M | Adrenal gland | 10 | 2.75 | No | Around blood vessels |
| 28 | 39 | M | Thyroid | 3 | 2.5 | No | NA |
| 29 | 59 | M | Cerebrum | 95 | 2.75 | No | Some staining artifact |
| 30 | 1 | M | Cerebellum | 95 | 2.75 | No | NA |

TABLE 8
Summary of Tour of Tumor analysis for Protocol 1

| | | | | | PGP9.5 | | | |
|---|---|---|---|---|---|---|---|---|
| | | | | | Percent | PGP9.5 | aSyn | |
| | | | | | Positive | Average | Staining | |
| No. | Age | Sex | Organ | Diagnosis | Cells | intensity | present | Comments |
| 31 | 57 | M | Skin | squamous cell | 1 | 2 | No | Around blood vessels |
| | | | | carcinoma | | | | |
| 32 | 64 | F | Skin | malignant melanoma | NA | NA | NA | Abundant endogenous pigment |
| | | | | | | | | precludes evaluation |
| 33 | 36 | M | Subcutis | liposarcoma | <1 | 1 | No | Rare lipocyte with staining |
| 34 | 34 | F | Breast | ductal carcinoma | <1 | 2 | No | Around blood, vessels |
| | | | | in situ | | | | |
| 35 | 35 | F | Breast | infiltrating duct | 1 | 1.5 | No | NA |
| | | | | carcinoma | | | | |
| 36 | 33 | F | Lymph node | Hodgkin lymphoma | 5 | 2 | No | Scattered non tumor |
| | | | | | | | | lymphocytes and around |
| | | | | | | | | blood vessels |
| 37 | 53 | F | Bone | osteosarcoma | <1 | 2 | No | Endogenous pigment present |
| 38 | 60 | M | Lung | adenocarcinoma | 0 | 0 | No | Abundant silver particles |
| 39 | 72 | M | Lung | squamous cell | 65 | 2.5 | No | Tumor cell staining; |
| | | | | carcinoma | | | | abundant silver particles |
| 40 | 61 | F | Liver | cholangiocarcinonia | 5 | 2 | No | stromal background present |
| 41 | 52 | M | Liver | hepatocellular | 0 | 0 | No | Silver particles present |
| | | | | carcinoma | | | | |
| 42 | 52 | M | Liver | metastatic | 5 | 2.5 | No | TC staining |
| | | | | adenocarcinoma | | | | |
| | | | | (from rectum) | | | | |
| 43 | 56 | M | Esophagus | squamous cell | 2 | 2.5 | No | Around blood vessels |
| | | | | carcinoma | | | | |
| 44 | 68 | F | Stomach | adenocarcinoma | <1 | 2 | No | Single cell with staining |
| 45 | 52 | M | Stomach | malignant lymphoma, | 5 | 2.75 | No | NA |
| | | | | diffuse large B cell | | | | |
| 46 | 51 | F | Stomach | signet ring cell | 10 | 2.75 | No | NA |
| | | | | carcinoma | | | | |
| 47 | 61 | M | Duodenum | gastrointestinal | 80 | 1 | No | TC staining and scattered |
| | | | | stromal tumor, | | | | cells (2%) with 2.5+ |
| | | | | malignant | | | | staining |
| 48 | uk | M | Descending colon | adenocarcinoma | 0 | 0 | No | Large amount of necrosis, |
| | | | | | | | | scattered silver particles |
| 49 | 73 | M | Rectum | adenocarcinoma | 5 | 1.5 | No | Scattered silver particles |
| 50 | 56 | M | Kidney | renal cell carcinoma | 1 | 2.5 | No | Around blood vessels and |
| | | | | | | | | scattered silver particles |
| 51 | 53 | M | Urinary bladder | transitional cell | 10 | 2.75 | No | Nerve staining |
| | | | | carcinoma | | | | |
| 52 | 70 | M | Prostate | adenocarcinoma | 5 | 2.75 | No | Around blood vessels and |
| | | | | | | | | nerve |
| 53 | 34 | M | Testis | seminoma | 10 | 1 | No | TC staining |
| 54 | 64 | F | Uterine cervix | squamous cell | 5 | 2.75 | No | Nerve staining and |
| | | | | carcinoma | | | | around blood vessels |
| 55 | 69 | F | Endometrium | adenocarcinoma | 60 | 3 | No | TC staining |
| 56 | 45 | F | Ovary | metastatic | 25 | 1 | No | NA |
| | | | | adenocarcinoma | | | | |
| | | | | (from stomach) | | | | |
| 57 | 15 | F | Ovary | mucinous | 10 | 1.5 | No | NA |
| | | | | cystadenocarcinoma | | | | |
| 58 | 43 | F | Ovary | serous cystadenoma | 3 | 1 | No | Around blood vessels |
| | | | | of low malignant | | | | |
| | | | | potential | | | | |
| 59 | 69 | F | Thyroid | papillary carcinoma | 1 | 1 | No | NA |

TABLE 9
Summary of Tour of Tumor analysis for Protocol 2

| | | | | | PGP9.5 | | | |
|---|---|---|---|---|---|---|---|---|
| | | | | | Percent | PGP9.5 | aSyn | |
| | | | | | Positive | Average | Staining | |
| No. | Age | Sex | Organ | diagnosis | Cells | intensity | present | Comments |
| 31 | 57 | M | Skin | squamous cell | 2 | 2.5 | No | Stromal cell staining |
| | | | | carcinoma | | | | |
| 32 | 64 | F | Skin | malignant melanoma | NA | NA | NA | Abundant endogenous pigment |
| | | | | | | | | precludes evaluation |
| 33 | 36 | M | Subcutis | liposarcoma | 0 | 0 | No | Single lipocyte with 1+ |
| | | | | | | | | membrane staining |
| 34 | 34 | F | Breast | ductal carcinoma | <1 | 2 | No | Around blood vessel |
| | | | | in situ | | | | |
| 35 | 35 | F | Breast | infiltrating duct | 3 | 2 | No | TC staining |
| | | | | carcinoma | | | | |
| 36 | 33 | F | Lymph node | Hodgkin lymphoma | 2 | 2.5 | No | NA |
| 37 | 53 | F | Bone | osteosarcoma | <1 | 2 | No | Endogenous pigment present |
| 38 | 60 | M | Lung | adenocarcinoma | 0 | 0 | No | Silver particles present |
| 39 | 72 | M | Lung | squamous cell | 60 | 2.5 | No | TC staining |
| | | | | carcinoma | | | | |
| 40 | 61 | F | Liver | cholangiocarcinoma | 2 | 2 | No | NA |
| 41 | 52 | M | Liver | hepatocellular | 1 | 2.5 | No | NA |
| | | | | carcinoma | | | | |
| 42 | 52 | M | Liver | metastatic | 10 | 2.5 | No | TC staining |
| | | | | adenocarcinoma | | | | |
| | | | | (from rectum) | | | | |
| 43 | 56 | M | Esophagus | squamous cell | 10 | 2.5 | No | stromal background present |
| | | | | carcinoma | | | | |
| 44 | 68 | F | Stomach | adenocarcinoma | 0 | 0 | No | NA |
| 45 | 52 | M | Stomach | malignant lymphoma, | 3 | 2.5 | No | NA |
| | | | | diffuse large B cell | | | | |
| 46 | 51 | F | Stomach | signet ring cell | 10 | 2.75 | No | NA |
| | | | | carcinoma | | | | |
| 47 | 61 | M | Duodenum | gastrointestinal | 65 | 1.5 | No | TC staining 1.5+ |
| | | | | stromal tumor, | | | | scattered cells 2.5+ |
| | | | | malignant | | | | |
| 48 | uk | M | Descending colon | adenocarcinoma | 0 | 0 | No | Large amount of necrosis, |
| | | | | | | | | silver particles present |
| 49 | 73 | M | Rectum | adenocarcinoma | 10 | 2.5 | No | NA |
| 50 | 56 | M | Kidney | renal cell carcinoma | 2 | 2.5 | No | NA |
| 51 | 53 | M | Urinary bladder | transitional cell | 10 | 2.75 | No | Around blood vessels and |
| | | | | carcinoma | | | | nerve |
| 52 | 70 | M | Prostate | adenocarcinoma | 5 | 2.75 | No | Around blood vessels |
| 53 | 34 | M | Testis | seminoma | 10 | 2 | No | TC staining |
| 54 | 64 | F | Uterine cervix | squamous cell | 3 | 2.75 | No | Nerve staining; tissue |
| | | | | carcinoma | | | | tearing/folding |
| 55 | 69 | F | Endometrium | adenocarcinoma | 60 | 3 | No | TC staining |
| 56 | 45 | F | Ovary | metastatic | 3 | 1 | No | NA |
| | | | | adenocarcinoma | | | | |
| | | | | (from stomach) | | | | |
| 57 | 15 | F | Ovary | mucinous | 20 | 1 | No | NA |
| | | | | cystadenocarcinoma | | | | |
| 58 | 43 | F | Ovary | serous cystadenoma | 5 | 2 | No | Scattered stromal cells |
| | | | | of low malignant | | | | |
| | | | | potential | | | | |
| 59 | 69 | F | Thyroid | papillary carcinoma | 1 | 1 | No | NA |

### Example 2. Performance of 7E2 Compared with Other pS129-aSyn Antibodies for Immunohistochemical Staining of FFPE Tissue

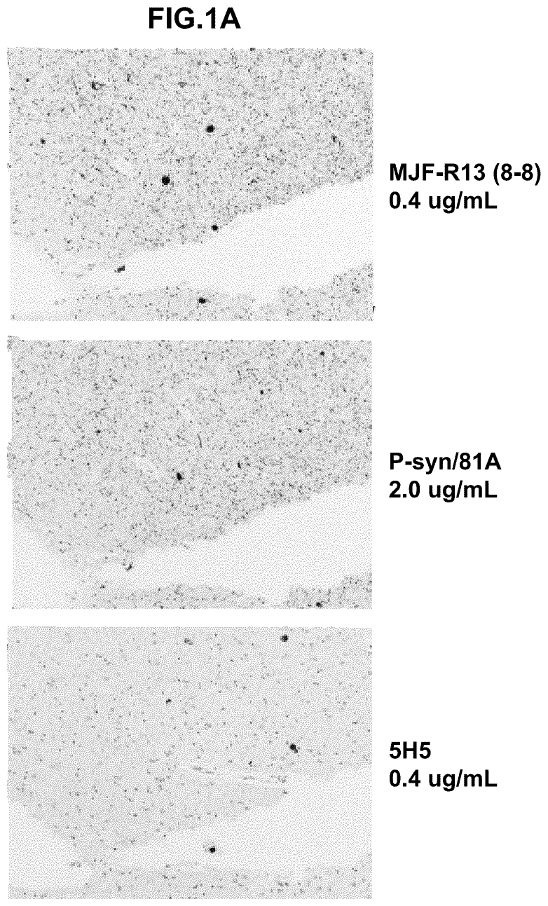

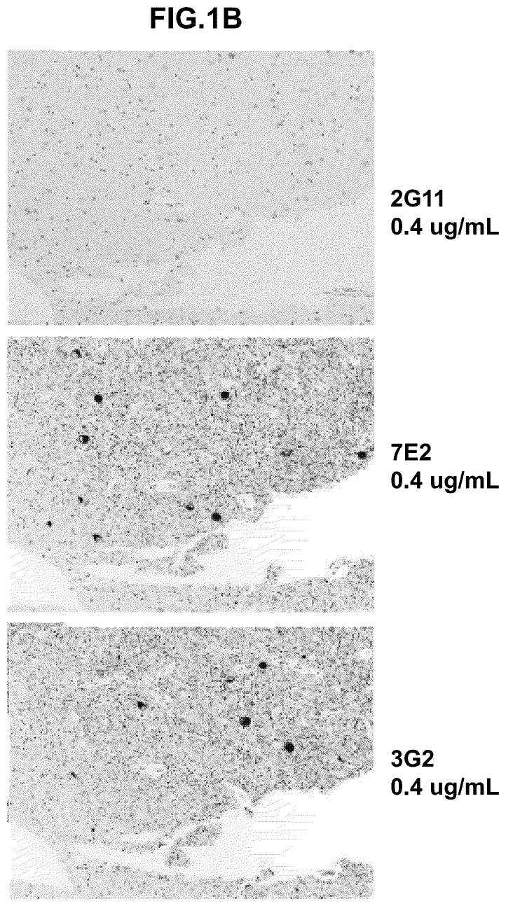

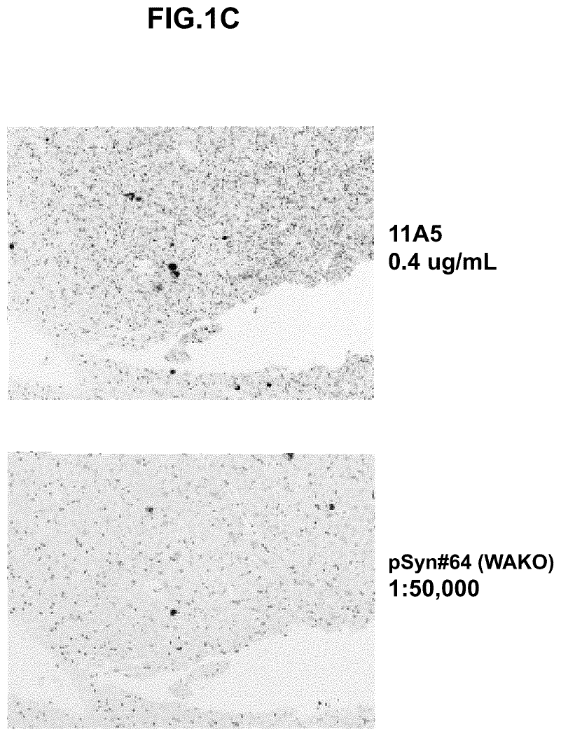

Detection of aggregated alpha-synuclein based on differential sensitivity of aggregated and non-aggregated alpha-synuclein to proteolytic degradation provides the basis for the identification of Lewy bodies in brain and definitive diagnosis of Parkinson's disease. To assess the ability of antibodies against phosphorylated S129 alpha-synuclein including 7E2 to detect Lewy bodies, cortical brain sections from two donors with Parkinson's disease were stained using VENTANA® OptiView DAB IHC Detection Kit. All 8 anti-phosphorylated S129 alpha-synuclein antibodies (81A, #64, MJF-R13(8-8), 5H5, 2G11, 7E2, 3G2, and 11A5) produced DAB stains in PD brain sections that were characteristic of Lewy bodies (see Table 10 for list of non-limiting, exemplary alpha-synuclein antibodies tested). Of the 8 anti-phosphorylated alpha-synuclein antibody clones tested, 7E2, 3G2, 11A5, and MJF-R13(8-8) exhibited higher sensitivity for pS129-aSyn (see FIG. 1A-1C).

TABLE 10
List of non-limiting, exemplary alpha-synuclein antibodies

| | | | | Selected | |
|---|---|---|---|---|---|
| | | | | [conc.] | |
| Antibody | Clone | Species | Source | for FFPE | |
| a-syn | LB 509 | Ms | Abcam | 1.0 | μg/mL |
| a-syn | 5C12 | Ms | Prothena | 0.5 | μg/mL |
| Phospho a-syn | MJF-R13 (8-8) | Rb | Abcam | 0.4 | μg/mL |
| Ab168381 | | | | | |
| Phospho-a-syn | P-syn/81A | Ms | Abcam/Biolegend/Millipore | 2.0 | μg/mL |
| Phospho-a-syn | 5H5 | Rb | Roche Diagnostics GmbH | NA | |
| Phospho-a-syn | 2G11 | Rb | Roche Diagnostics GmbH | NA | |
| Phospho-a-syn | 7E2 | Rb | Roche Diagnostics GmbH | 0.08 | μg/mL |
| Phospho-a-syn | 3G2 | Rb | Roche Diagnostics GmbH | 0.08 | μg/mL |
| Phospho-hu-a-syn | 11A5 | Ms | Roche Diagnostics GmbH | 0.4 | μg/mL |

| | | | | |
|---|---|---|---|---|
| Phospho-a-syn | pSyn#64 | Ms | WAKO | NA |

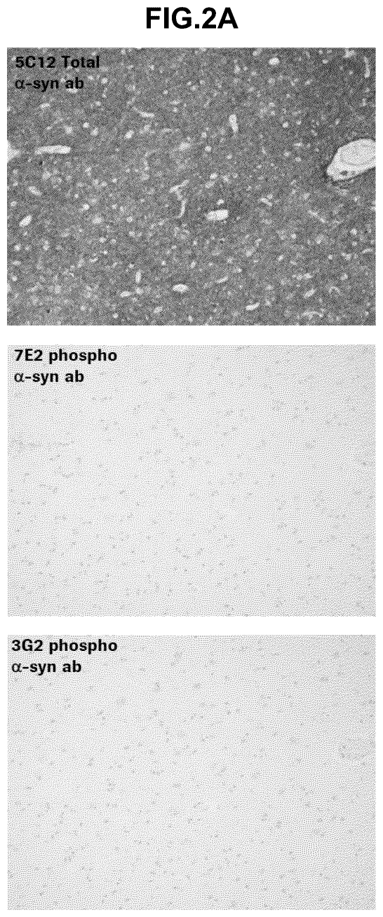

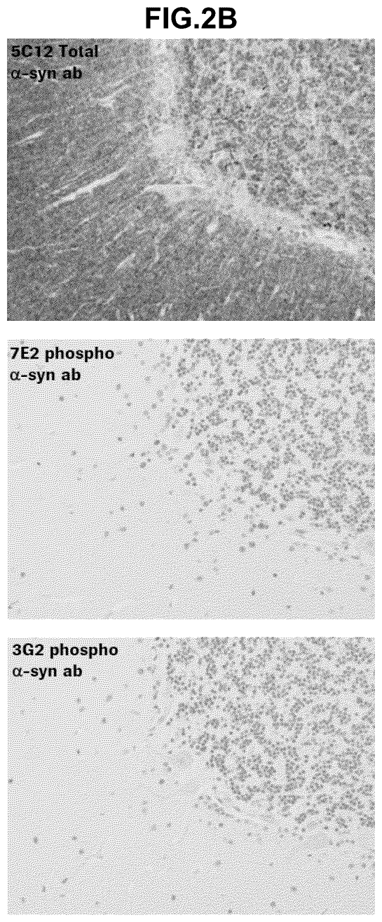

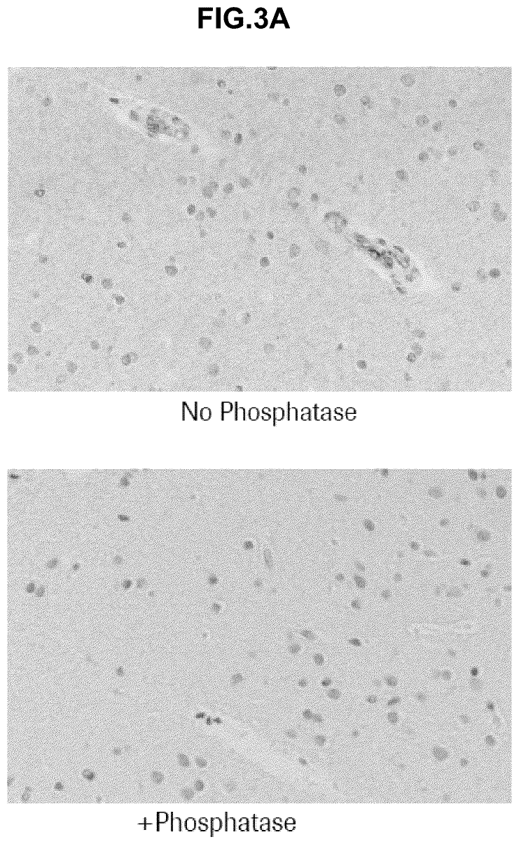

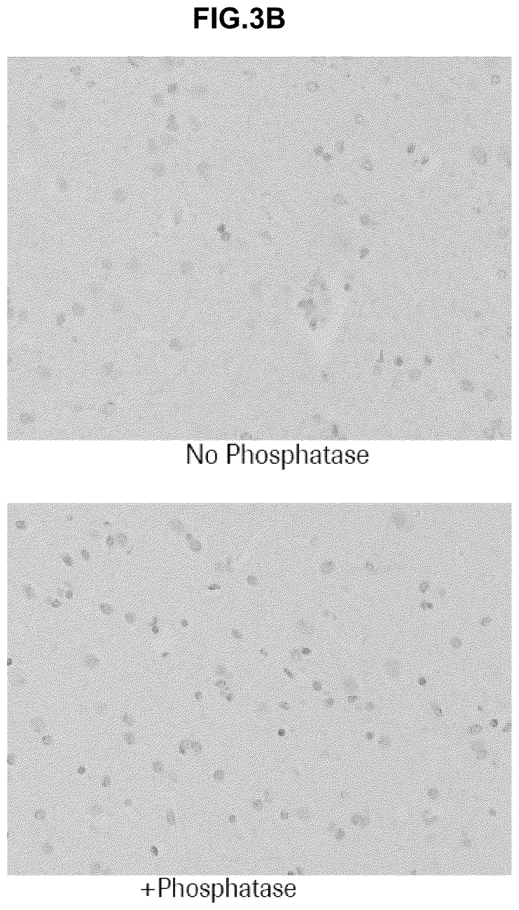

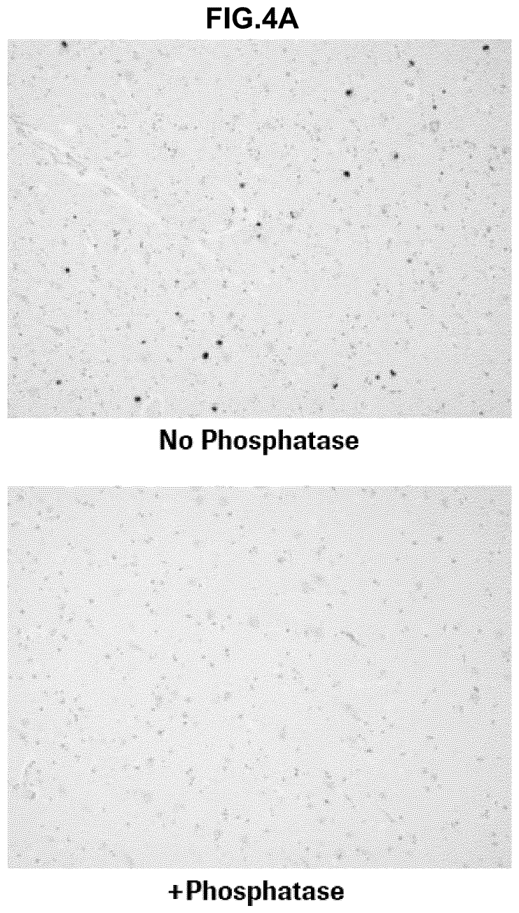

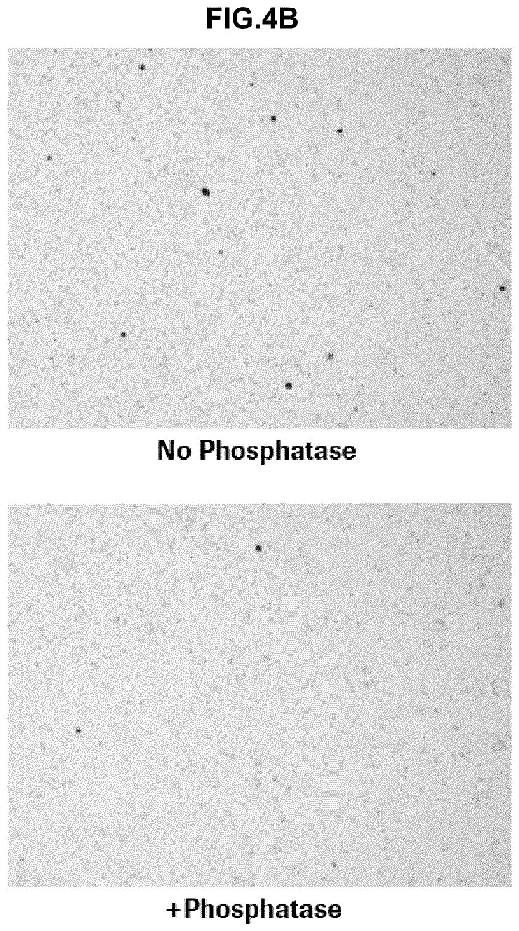

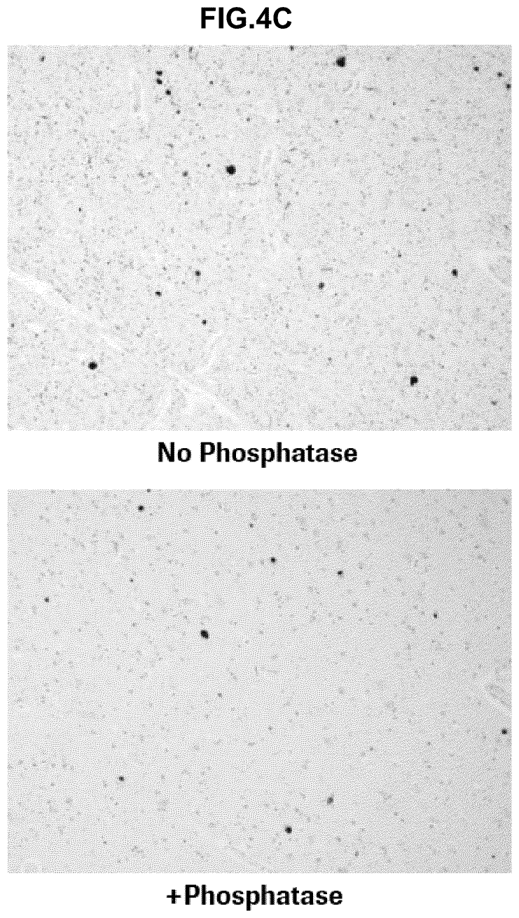

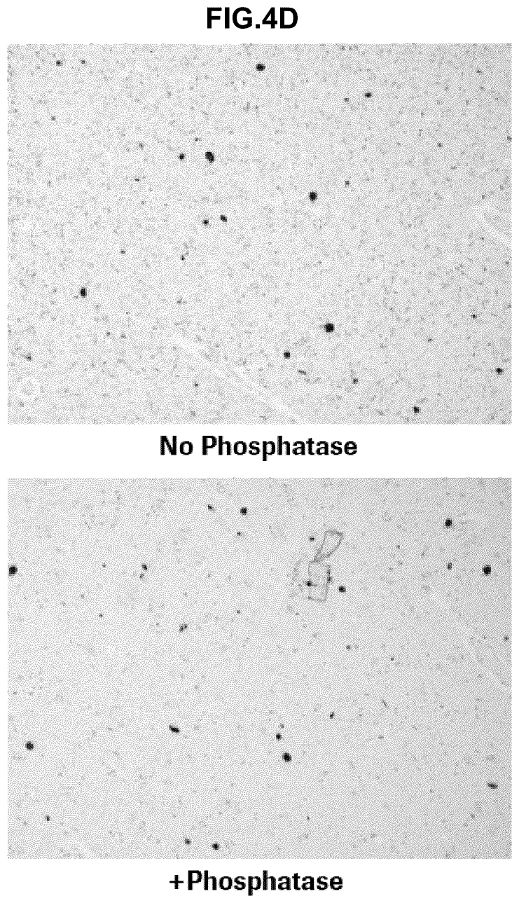

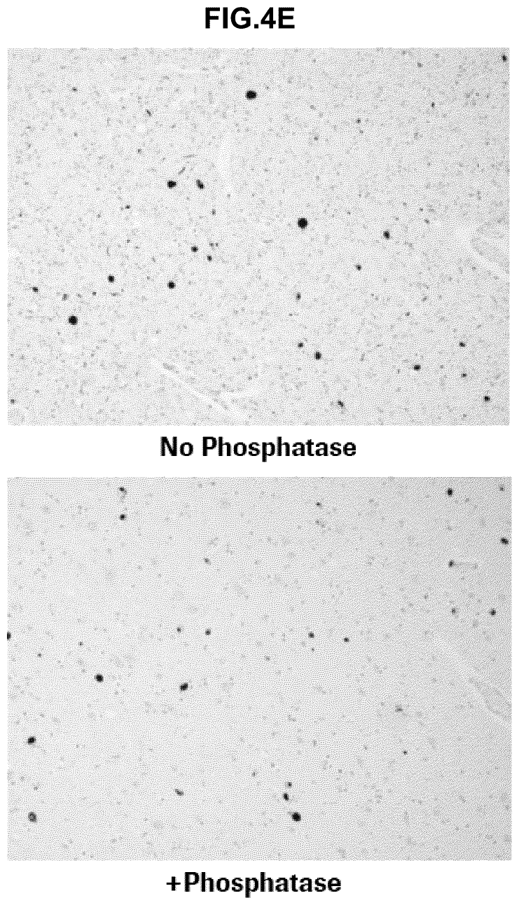

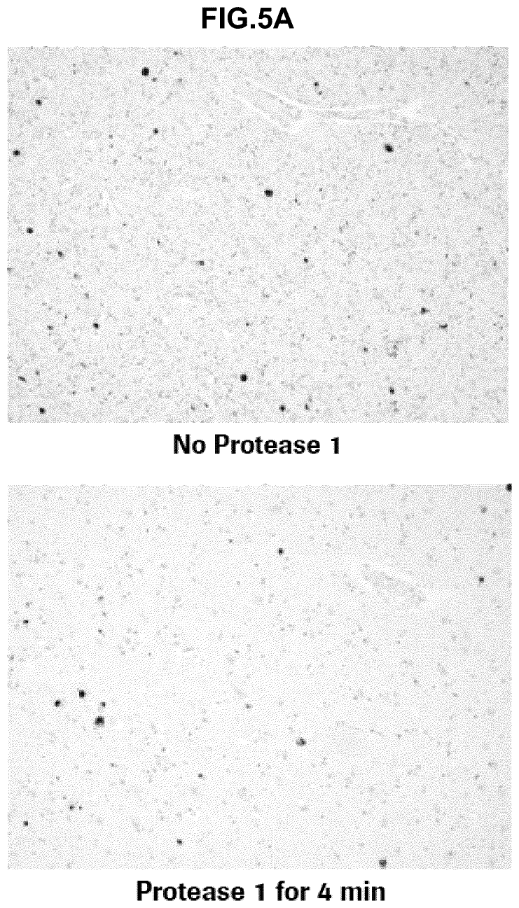

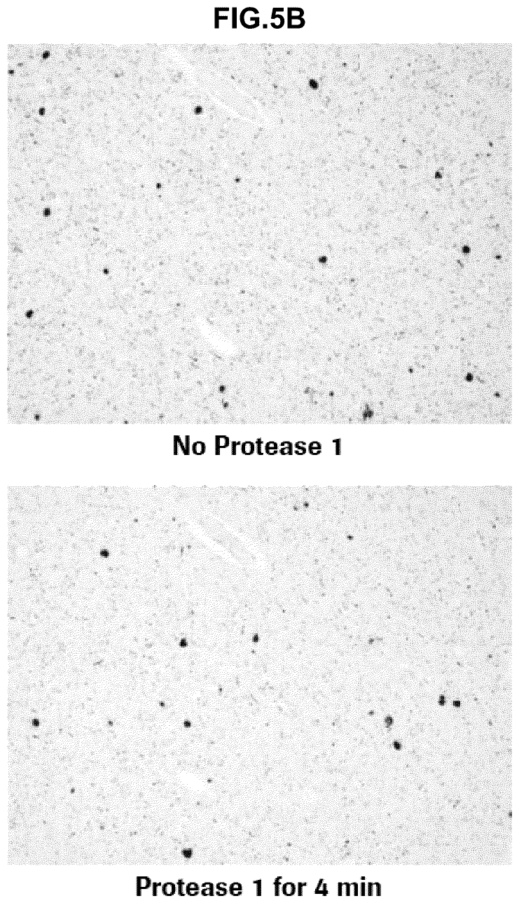

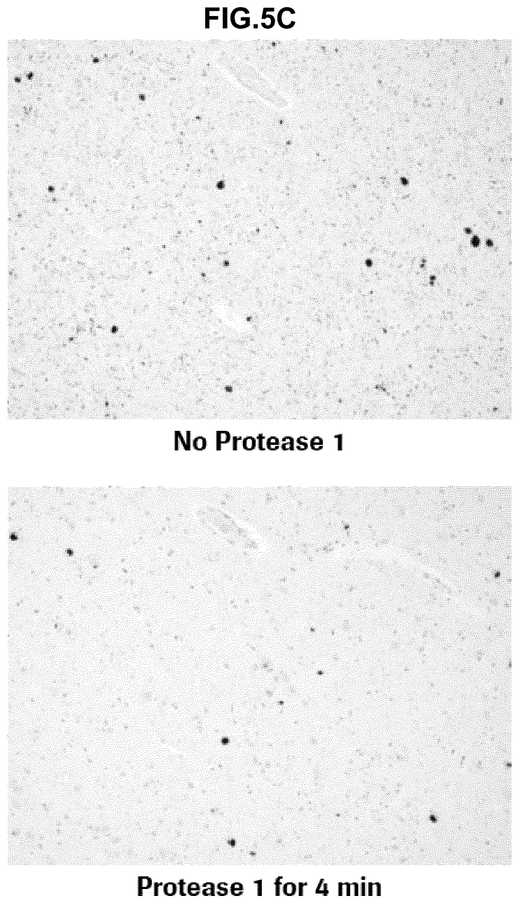

In brain sections from non-PD individuals no staining was observed with 3G2 and 7E2 antibodies without antigen retrieval (see FIGS. 2A and 2B). With ULTRA CC1 antigen retrieval, 3G2 and 7E2 antibodies produced faint cytoplasmic stains that were absent when samples were treated with phosphatase (see FIGS. 3A and 3B). Specificity of 81A, 7E2, 3G2, 11A5, and MJF-R13(8-8) for pS129-aSyn aggregated in Lewy bodies was tested by prolonged phosphatase or moderate protease treatments. Following 2 hours in 8.8 μg/mL highly active recombinant alkaline phosphatase at 36° C., extent of Lewy body staining in cortical brain sections from a PD donor was either severely (with MJF-R13(8-8) and 81A) or moderately (with 11A5, 7E2, and 3G2) decreased (see FIG. 4A-FIG. 4E). Thus phosphatase treatment appears to reduce non-Lewy body staining without significantly affecting Lewy body staining. In contrast, treatment with VENTANA™ Protease 1 at 36° C. for 4 minutes had negligible effect Lewy body staining produced by 11A5, 7E2, or 3G2 antibodies in PD brain sections (see FIG. 5A-FIG. 5C). As control, the same protease treatment removed most alpha-synuclein staining produced by two different antibodies against S129-phosphorylated or non-S129 phosphorylated aSyn (LB509 and 5C12 monoclonal antibodies) in PD brain except for Lewy bodies (data not shown).

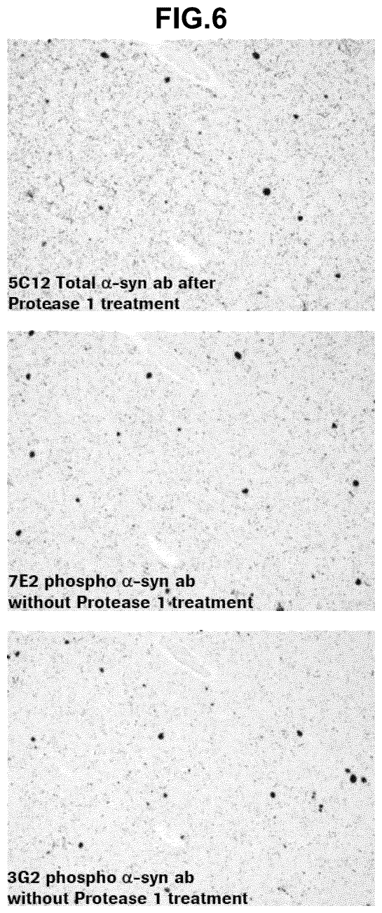

Overall, phosphorylation specific alpha-synuclein antibodies 7E2 and 3G2 in absence of protease treatment produced staining patterns in brain sections from a subject with PD that were identical to total alpha-synuclein antibody 5C12 after protease treatment (see FIG. 6). However, 3G2 generally exhibited higher background than 7E2 (data not shown). In addition to poorer staining performance, Abcam's 81A and WAKO's #64 exhibited lower specificity compared with the other anti-phosphorylated S129 alpha-synuclein antibodies. Unlike 3G2 and 7E2, 81A led to neurite staining in brain section from a non-PD individual (data not shown). This may reflect the reported cross-reactivity of 81A for neurofilament light (NFL) phosphorylated at Ser473. At low titer (1:1000), #64 antibody clone from WAKO led to non-specific nuclear staining in brain sections from both PD and non-PD subjects that persisted after phosphatase treatment and was not observed with the other 7 antibody clones (data not shown).

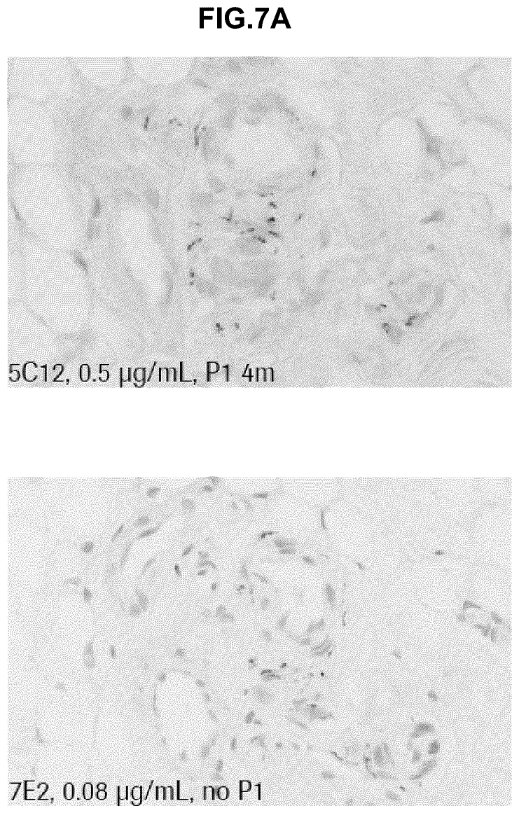

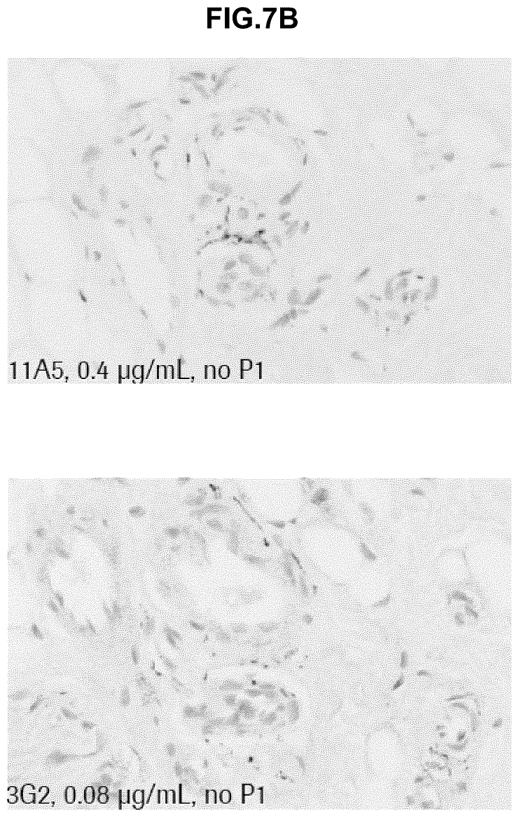

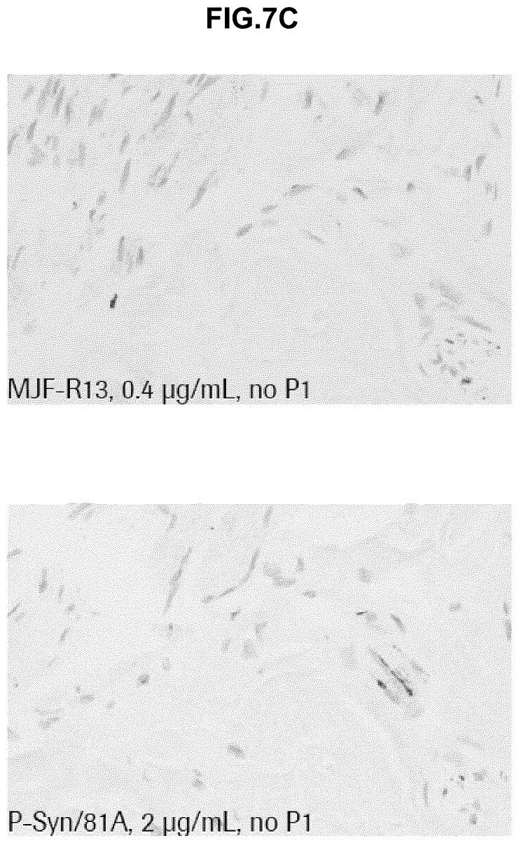

In addition to brain, the ability of anti-pS129-aSyn antibody clones to detect aggregated aSyn in skin samples from subjects with PD was assessed. In absence of protease-based antigen retrieval, all of the anti-pS129-aSyn antibodies were able to detect aggregated aSyn in skin section from a subject with PD similar to that detected using the anti-total aSyn 5C12 antibody coupled to VENTANA™ Protease 1 treatment (see FIG. 7A-FIG. 7C). Antibody clones 81A, 7E2, and MJF-R13(8-8) were further tested on skin sections from subjects with PD and non-PD control subjects. 3G2 was not tested due to its similar staining intensity and pattern and higher background compared with 7E2 antibody on brain samples. Staining with MJF-R13(8-8) was associated with non-specific background in smooth muscle and collagen while 81A led to non-specific nuclear staining (data not shown).

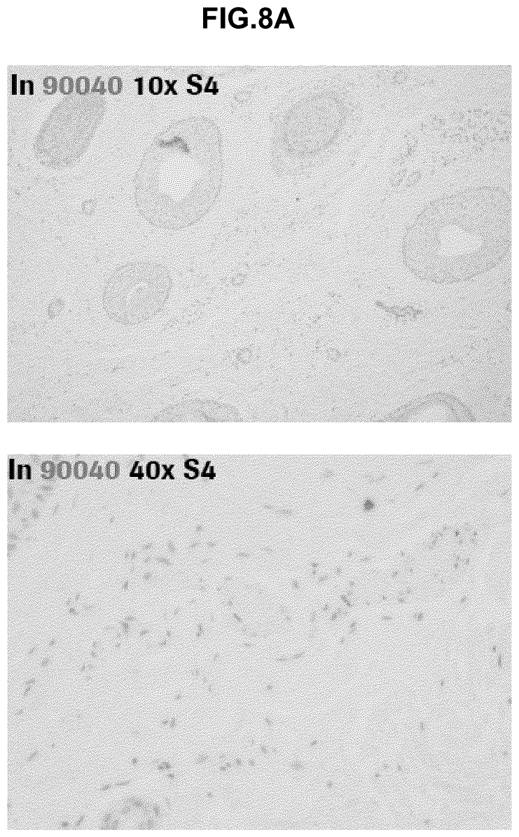

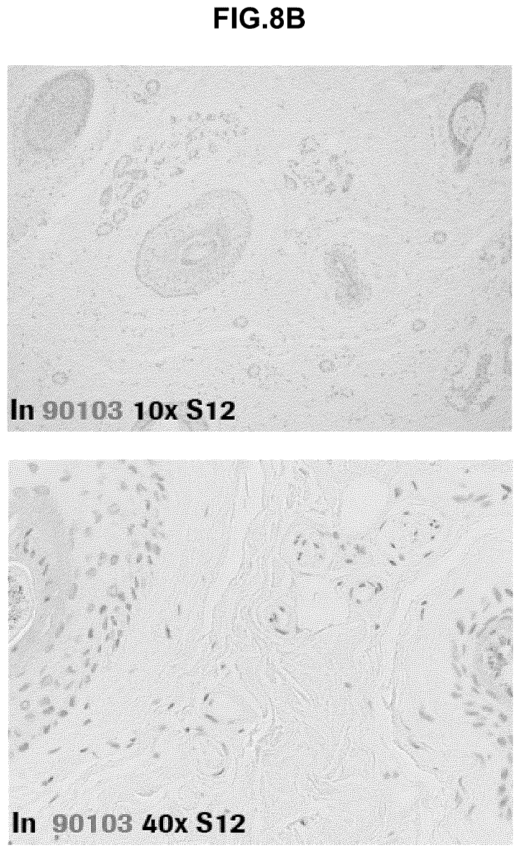

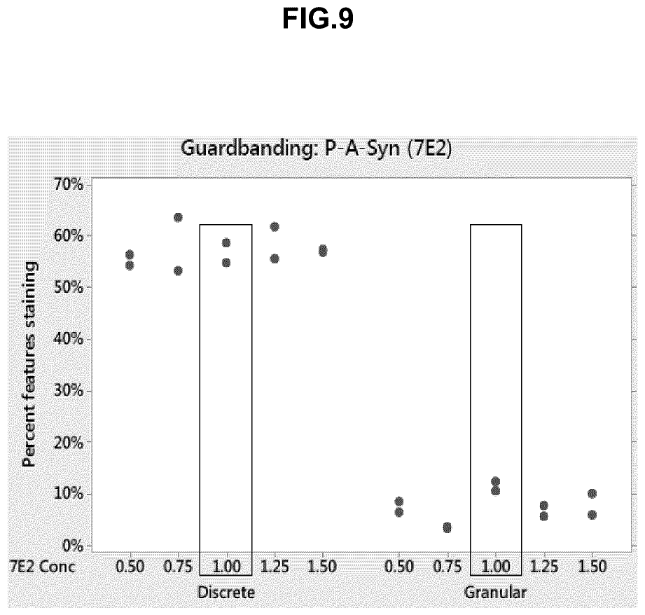

Diluent and titration experiments were performed to optimize phosphorylated S129 alpha-synuclein staining using 7E2 antibody. Results of diluent testing showed diluent 90040 and diluent 90103 to have minimal background while preserving staining intensity (see FIG. 8A and FIG. 8B). Titration experiments showed 7E2 dispenser concentration of 1 μg/mL to have near maximal staining intensity and minimal background (data not shown). The titration experiments results were reinforced by those of guardbanding experiments in which quantitative measurements in the form of percent neuronal features stained per slide were performed (see FIG. 9). Accelerated stability testing performed using both duplex silver/yellow and single-plex DAB detection schemes showed 7E2 to have a predicted shelf-life of 24 months at 4° C. (data not shown).

### Example 3. Selection of PGP9.5 Antibody for Immunohistochemical Staining

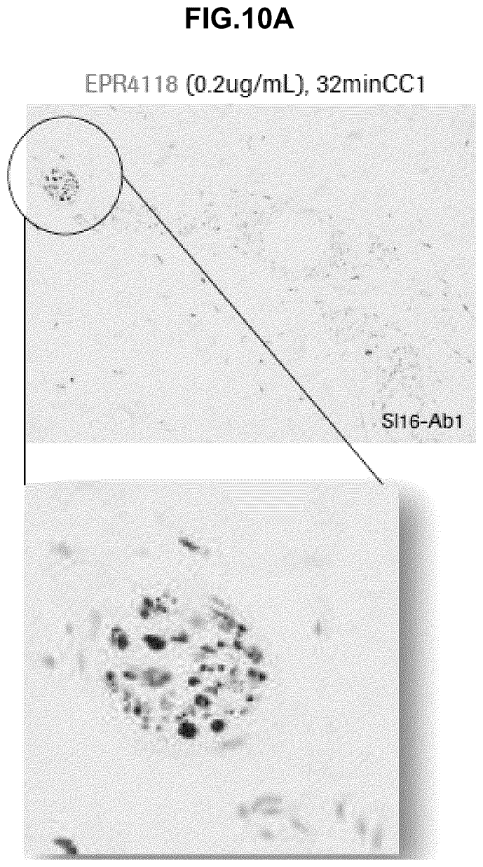

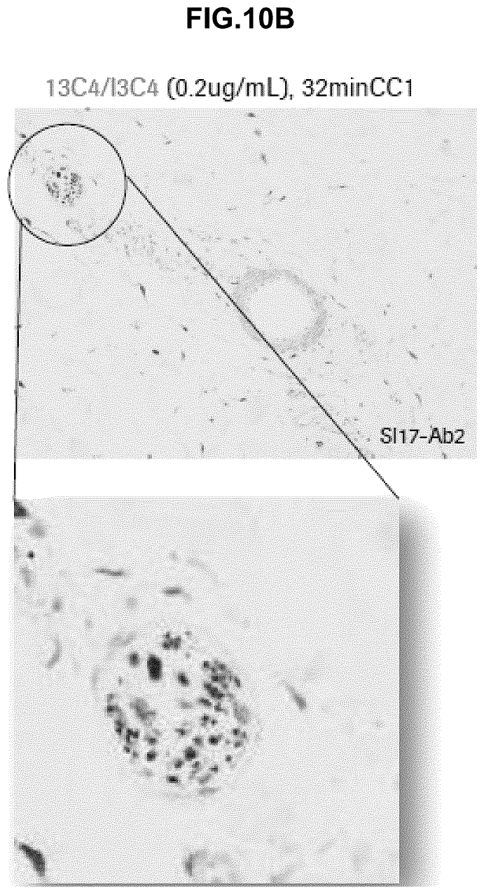

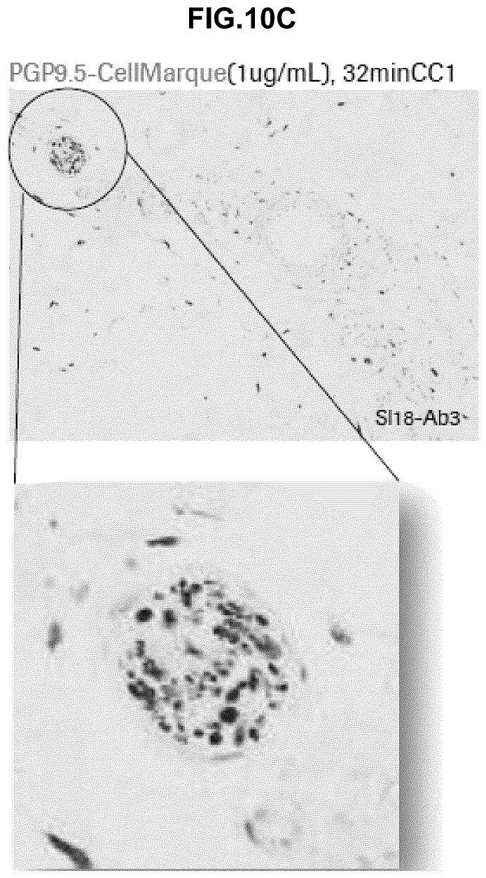

Protein gene product 9.5 (PGP9.5), more descriptively known as ubiquitin carboxyl-terminal hydrolase L1 (UCHL1), is a thioesterase essential for de-ubiquitination of ubiquitin-conjugated proteins and maintenance of free mono-ubiquitin pool 20-24. It is highly expressed in the brain and has been estimated to comprise 1% to 5% of total neuronal protein. PGP9.5 is commonly used as a marker for neurons and neuroendocrine cells, but the expression of PGP9.5 is not exclusively restricted to those cell types. Three different antibodies against PGP9.5 were evaluated for immunohistochemical detection of nerve features, neurons, or neuroendocrine cells in skin, a polyclonal antibody available in RTD catalog (P/N 760-4434) from Cell Marque™ and two monoclonal antibodies from Abcam, a rabbit (EPR4118) and a mouse (13C/I3C4). All three antibodies were able to specifically stain structures that were recognizably neuronal (see FIG. 10A-FIG. 10C). Following a titration series of three antibody concentrations, EPR4118 and 13C/13C4 at 0.2 μg/mL were shown to have comparable staining intensity as polyclonal antibody from Cell Marque at dispenser concentration of 1.13 μg/mL (see FIG. 10A-FIG. 10C). However, at these concentrations the 13C/13C4 antibody exhibited significant amount of non-specific background staining compared with EPR4118 or Cell Marque™ polyclonal antibodies (compare FIG. 10B to FIG. 10A and FIG. 10C).

Specificity of EPR4118 antibody in the immunohistochemical context of phospho-S129-alpha-synuclein and PGP9.5 silver/yellow dual IHC assay was assessed using BC8 human tissue microarray containing 30 normal and 29 cancer tissue cores from various anatomical sites (SuperBioChips Laboratories, Seoul, Korea). The ToB/ToT analysis demonstrates PGP9.5 staining around blood vessels and glands and in nerve and ganglion cells from various tissue sites. Specialized structures with nerve innervation (pancreatic islets) also stained positive for PGP9.5 (see Tables 6-9). PGP9.5 staining was also observed in kidney tubules, rare stromal cells in lung, germ cells in testis, trophoblast cells in placenta, and tumor cells from various types of cancer. These findings are consistent with previously reported expression of PGP9.5 in various types of tumors and non-neuronal cell types.

Results of diluent testing, titrations, guardbanding, and accelerated stability (data not shown) led to the dispenser formulation of EPR4118 at 0.5 μg/mL in DISCOVERY Goat Ig Block (VENTANA P/N 760-6008). In this formulation, EPR4118 is expected to have a shelf-life of 24 months at 4° C. based on accelerated stability testing. Results of the lot-to-lot testing (data not shown) demonstrated the performance of EPR4118 antibody is consistent across four different production lots generated by two different manufacturers (Abcam and Spring).

### Example 4. Selection of aSyn and PGP9.5 Assay and Detection Configuration

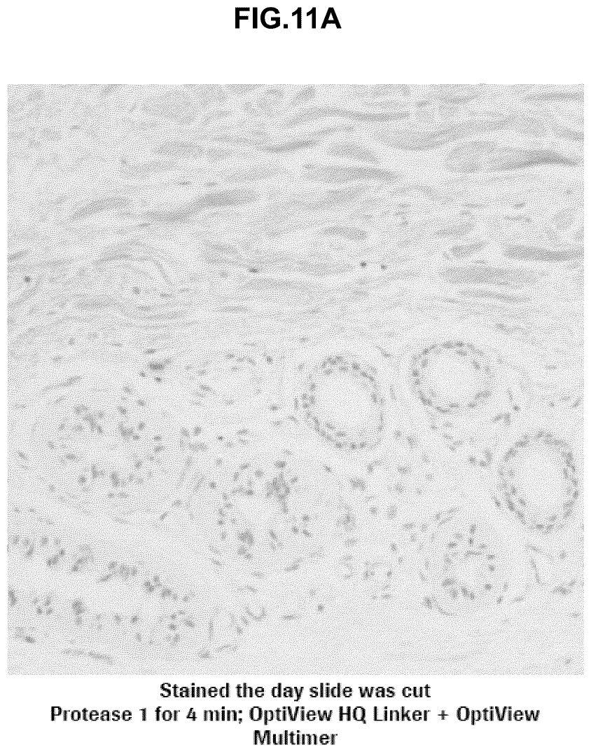

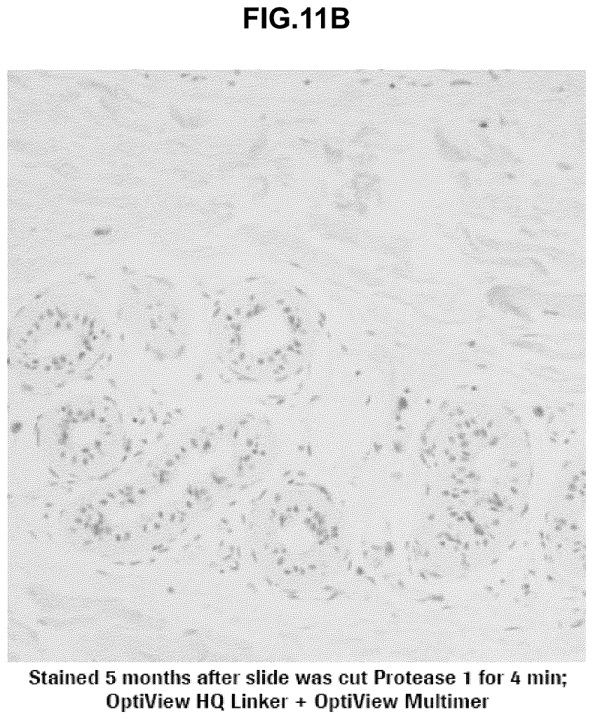

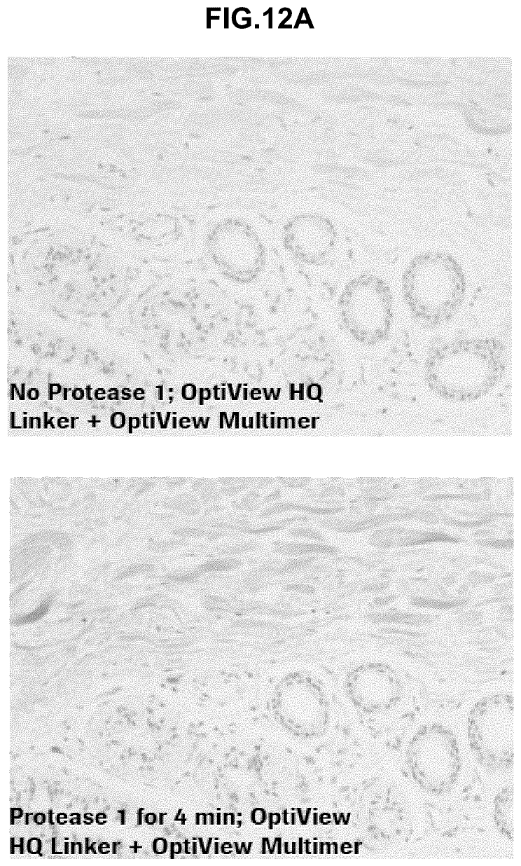

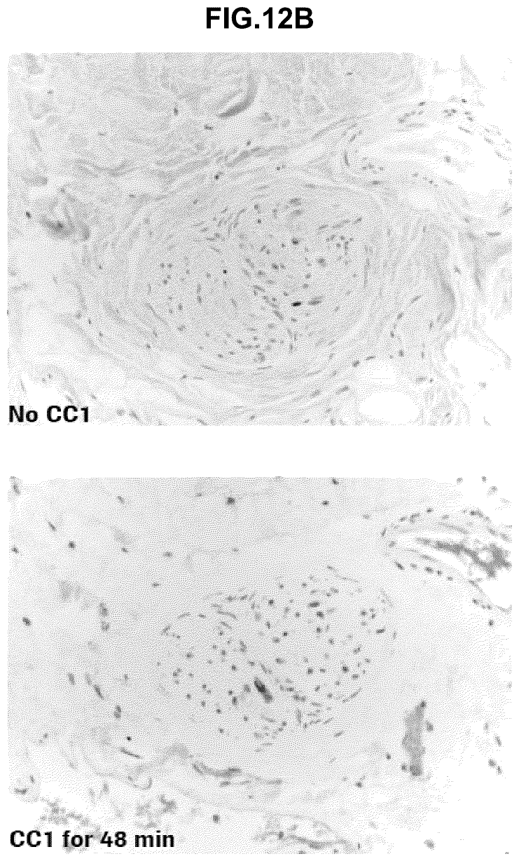

Protease resistance is a hallmark of many aggregated proteins including alpha-synuclein and detection of pathological forms of alpha-synuclein is enhanced with protease treatment. Roche pRED, in collaboration with Prothena, developed an assay (subsequently transferred to Targos) with high specificity toward aggregated alpha-synuclein in a cohort of PD and non-PD samples from BSHRI (see Table 4 and Table 5). The pRED/Prothena aSyn assay is based on the 5C12 antibody that recognizes both phosphorylated and unphosphorylated S129-aSyn, removal of non-aggregated aSyn by VENTANA® Protease 1, and detection using the highly sensitive VENTANA® OptiView DAB IHC Detection kit. However, while minimal background was observed with the pRED/Prothena aSyn assay protocol using skin sections that have been cut for at least several weeks, strong staining of collagen fibers was observed with freshly cut slides (see FIG. 11A and FIG. 11). The collagen background staining was exacerbated with additional antigen retrieval using Protease 1 (see FIG. 12A) but lessened with Ultra CC1 (see FIG. 12B). The cause of the collagen background staining was not 5C12 antibody as 7E2 antibody gave rise to the same collagen staining using the same detection scheme (data not shown). The background was also not due to non-specific adsorption of DAB by collagen fibers as ultraView DAB IHC Detection kit did not generate any collagen staining (data not shown), but deposition of tyramide-TAMRA (VENTANA Purple kit, DISCOVERY, P/N 760-229) using OptiView HQ Universal Linker and HRP Multimer did (data not shown). Eventually OptiView HRP Multimer was determined to be the cause of collagen fiber staining (data not shown). Physiological cross-linking of collagen fibers leads to formation of many types of adducts. It is possible the anti-HQ IgG cross-reacts with one or more of such adducts. This hypothesis is consistent with lessening of collagen background staining following reversal of collagen cross-linking associated with CC1 treatment.

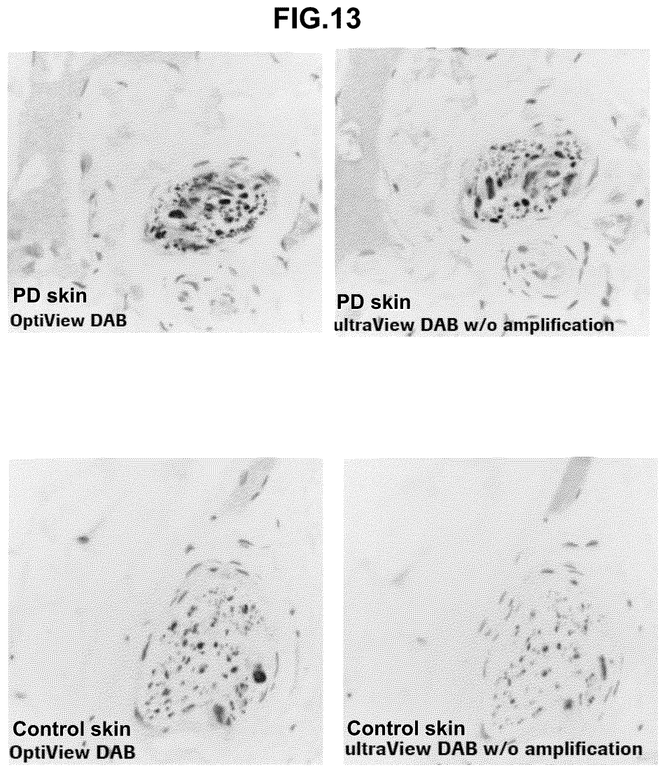

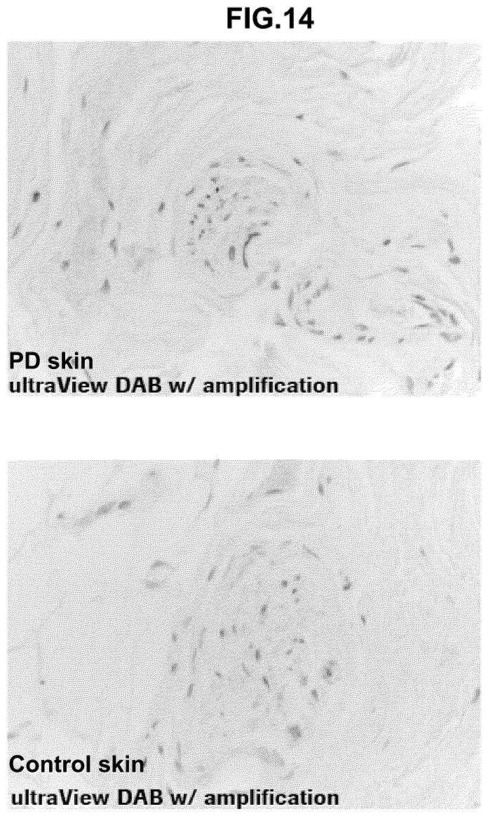

Survey of a collection of scalp samples from the BSHRI cohort showed varying degrees of collagen background DAB staining from light to sufficiently heavy to interfere with interpretation of specific signals from aggregated aSyn (data not shown). This prompted a search for alternative detection methods with comparable sensitivity as OptiView DAB IHC Detection Kit for staining of aggregated aSyn. As shown in FIG. 13, ultraView Universal DAB Detection Kit consistently gave lower staining intensity for PGP9.5 compared with OptiView DAB IHC Detection Kit. As a result, ultraView Universal DAB Detection Kit coupled to amplification using Ms anti-Rb IgG followed by Rb anti-Ms IgG (VENTANA® Amplification Kit, P/N 760-080) was used to stain pS129-aSyn. Even though ultraView Universal DAB Detection Kit with amplification may have similar staining intensity as OptiView DAB IHC Detection Kit, the amplification process gave rise to significant non-specific background (see FIG. 14). High background staining was also observed when the VENTANA iView DAB Detection Kit (P/N 760-091) was used to detect pS129-aSyn or PGP9.5 (data not shown).

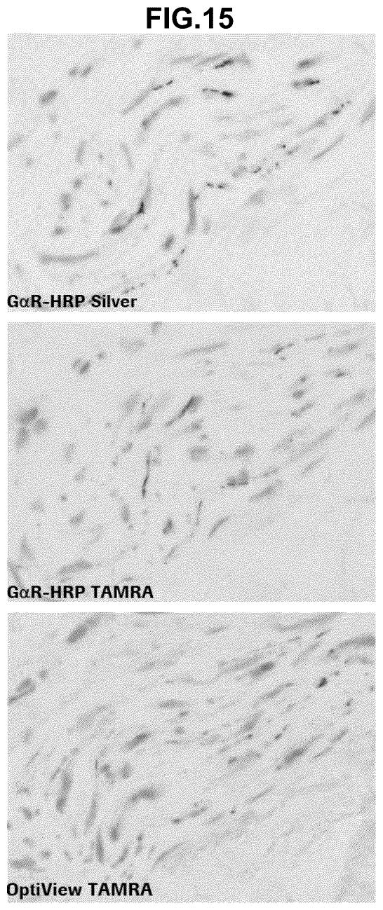

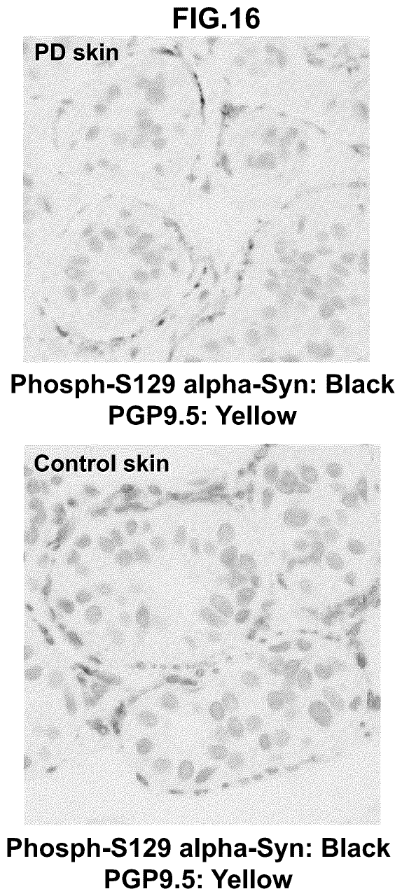

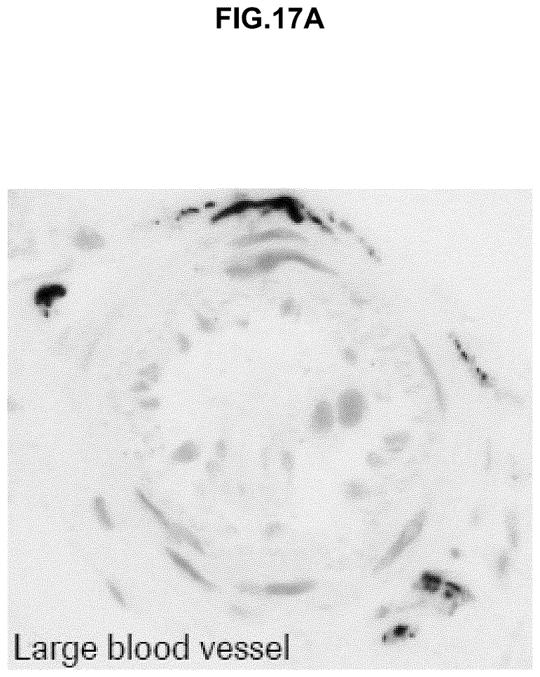

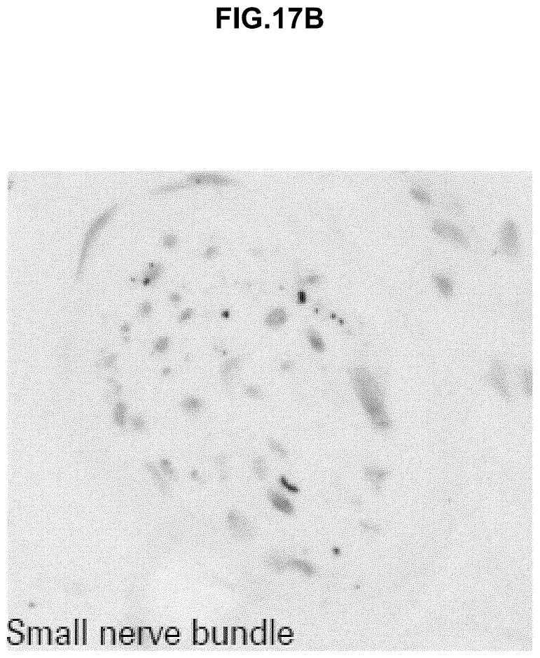

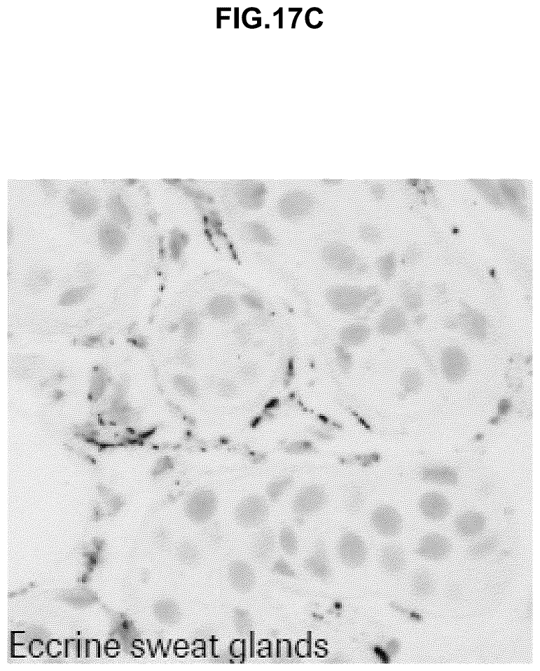

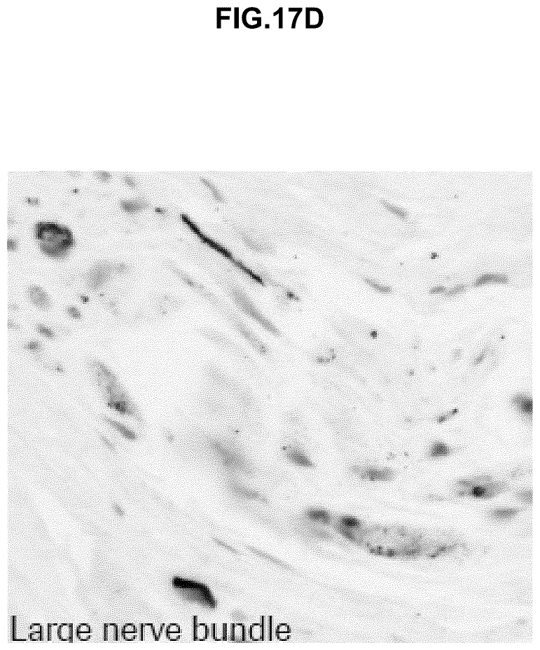

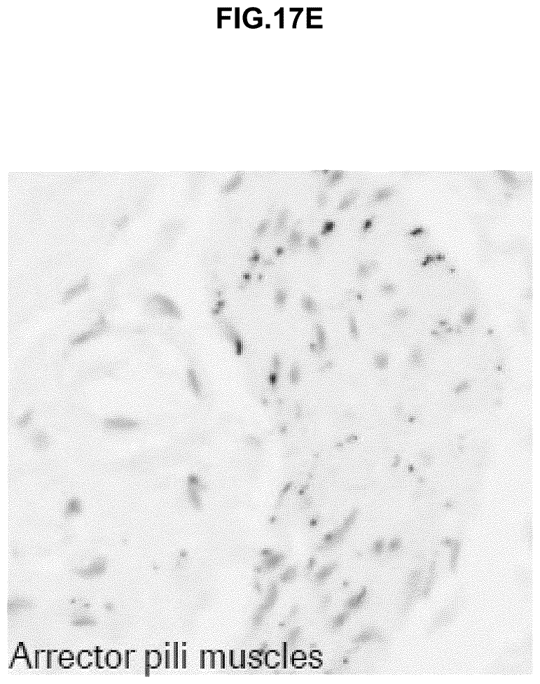

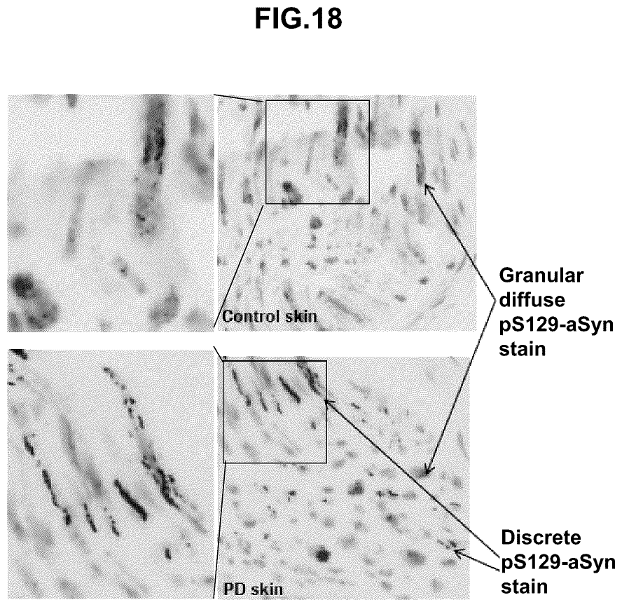

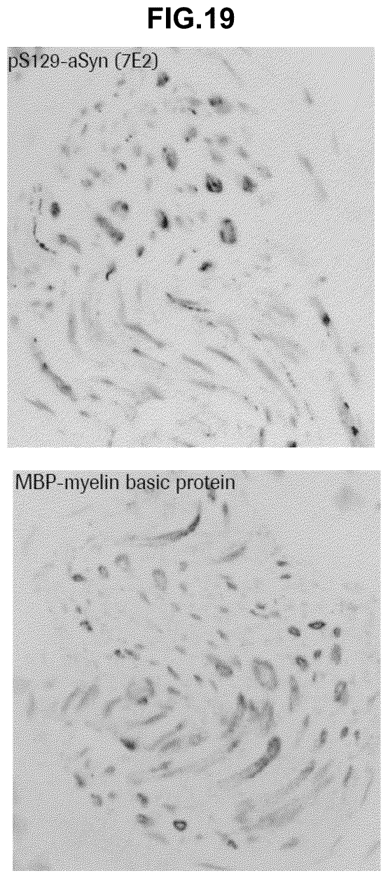

Two additional detection systems were evaluated for staining pS129-aSyn in skin sections using 7E2 antibody, ultraView SISH DNP Detection Kit (P/N 760-098) and DISCOVERY™ Purple kit (P/N 760-229). As shown in FIG. 15, both systems were able to detect aggregated pS129-aSyn with little to no background as long as goat anti-rabbit-HRP conjugate rather than OptiView HQ Universal Linker and HRP Multimer was used for stain deposition. In particular, the black silver stain deposited by the ultraView SISH DNP Detection Kit provided high-contrast visualization of aggregated pS129-aSyn in the background of PGP9.5 stained yellow using VENTANA® DISCOVERY™ UltraMap anti-Rb Alk Phos (P/N 760-4314) and QM-DABSYL as described in Methods section (see FIG. 16). The morphologies of silver/black pS129-aSyn and yellow PGP9.5 stains in different types of nerve features in skin from subjects with PD are shown in FIG. 17A-FIG. 17E. Using Bumblebee detection configuration, two different types of pS129-aSyn stains were observed in large nerve bundles of skin sections from subjects with PD: granular, diffuse-appearing stain and discrete, dense-appearing stain (see FIG. 18). As more fully described in subsequent sections, pS129-aSyn stain of the granular type was observed in FFPE skin sections from both subjects with PD and non-PD subjects in the BSHRI cohort. In contrast, pS129-aSyn stain of the discrete type was almost exclusively restricted to skin from subjects with PD. Subsequent sections also describe the sensitivity of the 7E2 antibody and optimized Bumblebee Assay protocols for aggregated aSyn in comparison with the pRED/Prothena assay based on 5C12 antibody, Protease 1, and OptiView DAB IHC Detection Kit. The granular pS129-aSyn stain often appears as bracketing axons in sagittal nerve sections (see FIG. 18) and as surrounding neurons circumferentially in transverse nerve sections (top panel of FIG. 19). Such staining pattern is similar to that of the Schwann cell marker myelin basic protein (bottom panel of FIG. 19), suggesting the pS129-aSyn protein responsible for the granular stain appearance could be located in Schwann cells. Accumulation of pS129-aSyn within Schwann cells has been previously described in patients with multiple system atrophy.

One assay configuration that was not tested is antigen retrieval with Protease 1 followed by incubation with 5C12 antibody and detection using ultraView SISH DNP Detection Kit. This assay configuration potentially has similar sensitivity for aggregated aSyn as 7E2 antibody detected using ultraView SISH DNP Detection Kit. However, it is not compatible with dual detection incorporating PGP9.5 as neuronal marker because PGP9.5 requires antigen retrieval using Cell Conditioning (CC) 1 or 2 bulk solutions and the combination of Protease 1 with CC1 or CC2 treatments leads to significant loss of tissue morphology (data not shown).

### Example 5. Optimization of Antigen Retrieval Conditions for Phospho-aSyn Staining Using 7E2 Antibody Clone and PGP9.5 Staining Using EPR4118 Antibody Clone

Immunohistochemical detections of many epitopes are enhanced following antigen retrieval procedures. Effects of two different antigen retrieval treatment processes on detection of pS129-aSyn in FFPE skin sections using 7E2 antibody were assessed: VENTANA® ULTRA Cell Conditioning (ULTRA CC1) bulk solution and protease. Effects of treatment with ULTRA CC1 or ULTRA CC2 solutions on PGP9.5 staining using EPR4118 antibody were also evaluated.

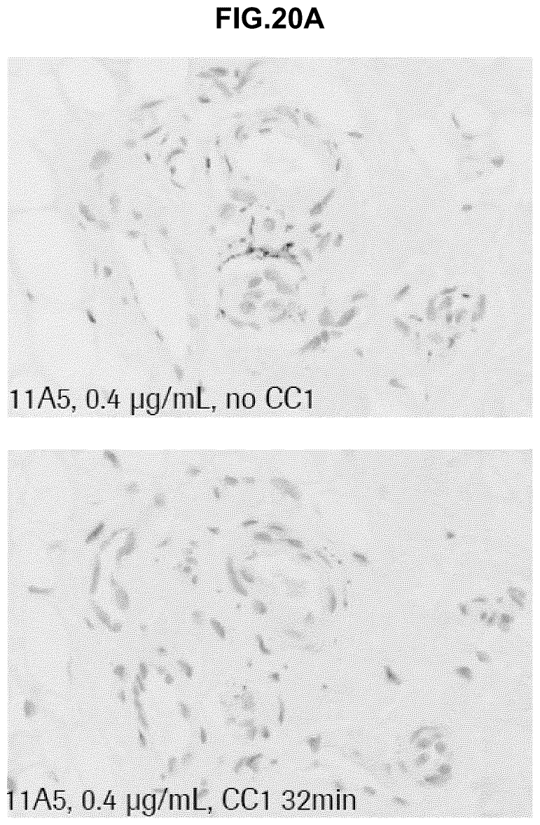

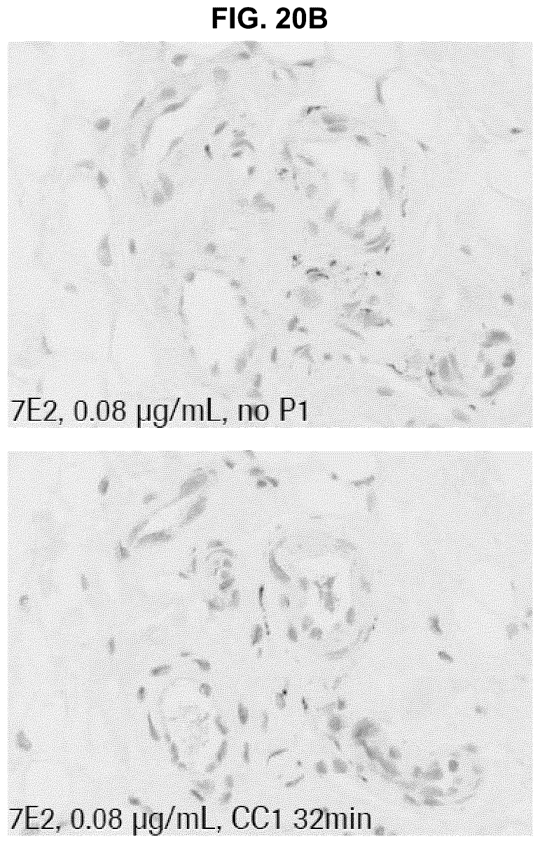

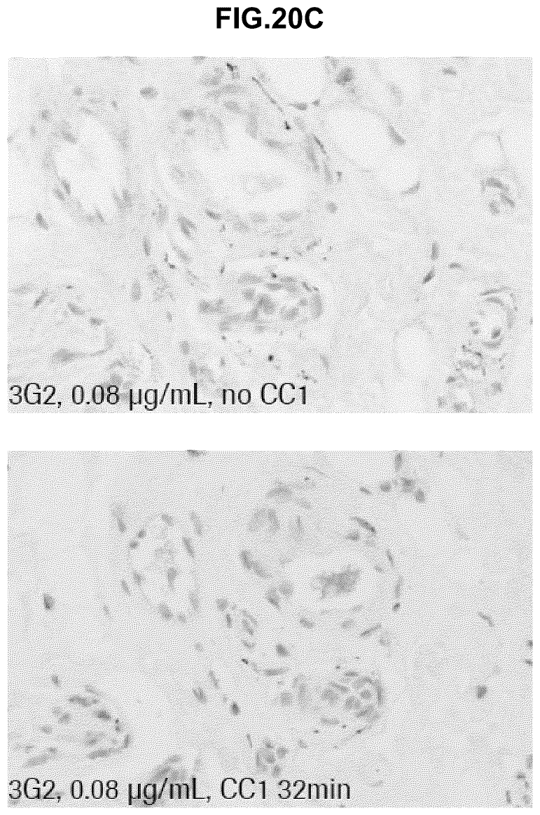

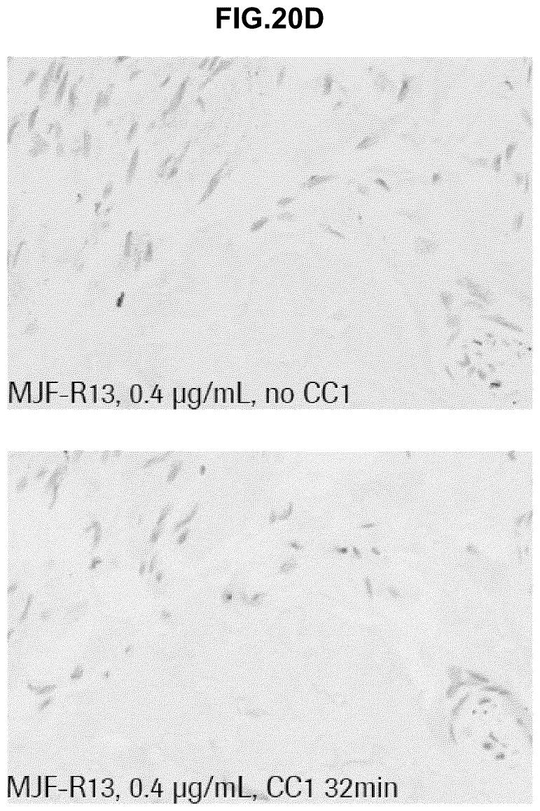

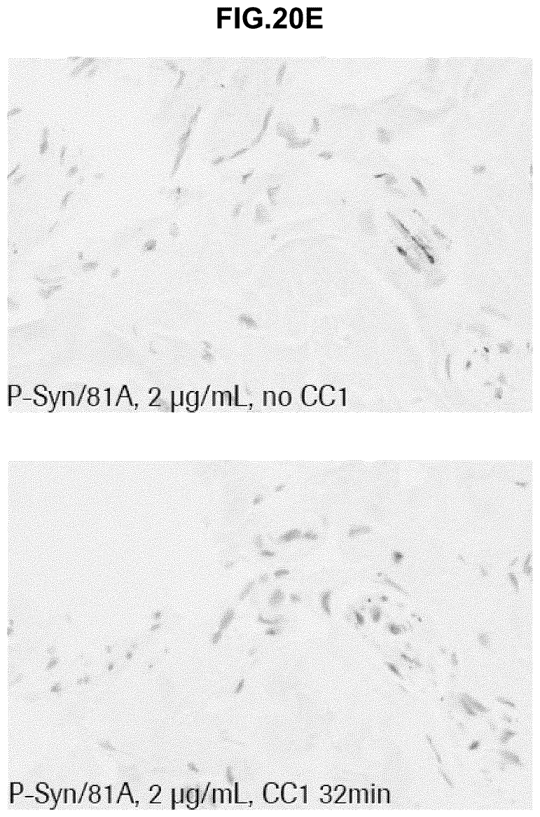

In contrast to most targets, staining of pS129-aSyn was paradoxically decreased after treatment with ULTRA CC1 for only 32 minutes at 100° C. (see FIG. 20A-FIG. 20E). The negative effect of ULTRA CC1 on pS129-aSyn staining was observed with five different antibodies against pS129-aSyn: 11A5, 7E2, 3G2, MJF-R13(8-8), and 81A (see FIG. 20A-FIG. 20E), suggesting the phosphorylation of S129 residue itself could have been affected by the treatment.

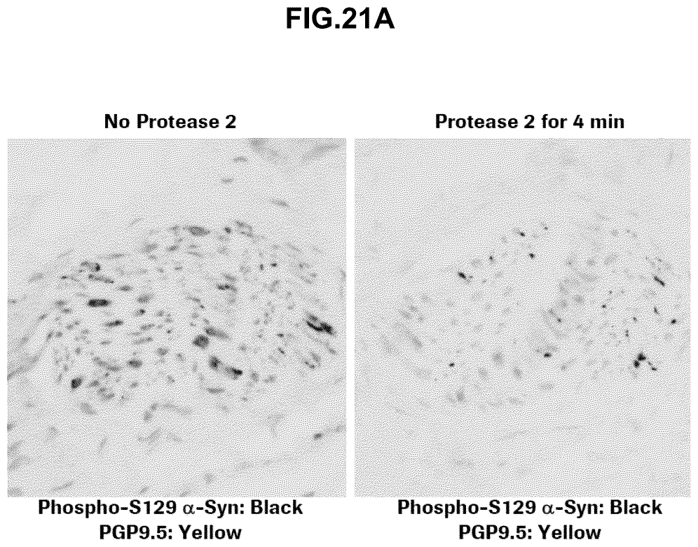

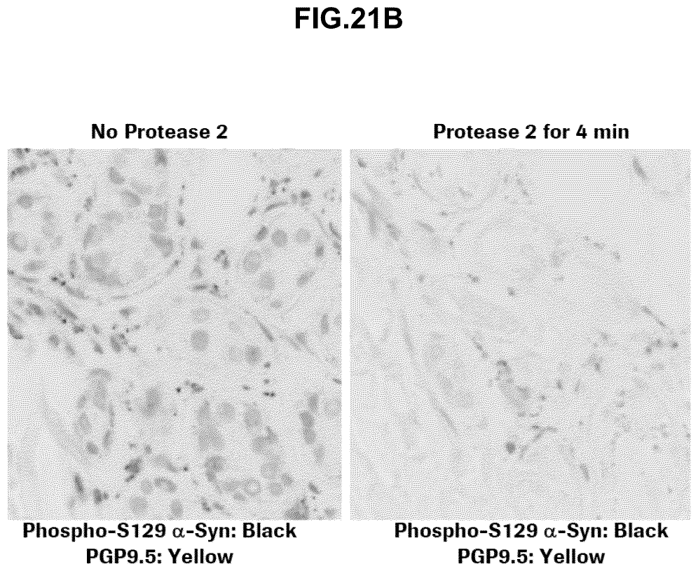

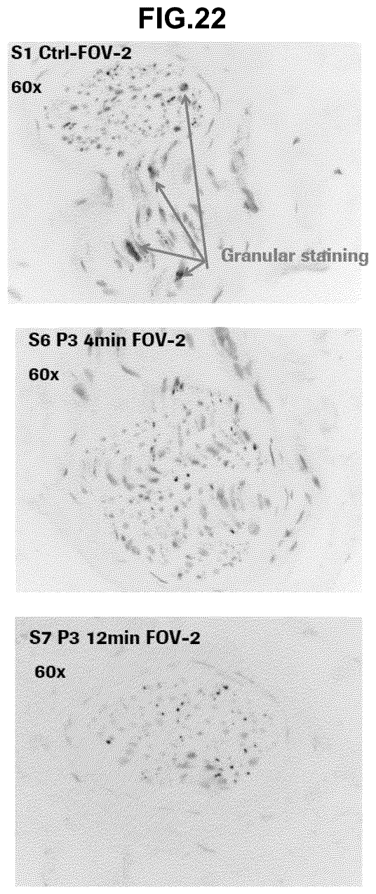

Protease treatment can improve detection of aggregated aSyn, but the underlying cause responsible for this phenomenon is unclear. It could be due to removal of un-aggregated aSyn or improved antibody access to epitopes buried in protein aggregates following protease digestion, or a combination of both. Treatment with VENTANA® Protease 2 for 4 minutes (see FIG. 21A and FIG. 21B) or VENTANA® Protease 3 for 4 minutes or for 12 minutes at 36° C. led to a moderate increase in the discrete type of pS129-aSyn stain in skin sections from subjects with PD (see FIG. 22). A more pronounced effect of protease treatment is the complete (Protease 2 for 4 minutes or Protease 3 for 12 minutes) or partial (Protease 3 for 4 minutes) removal of the granular type of pS129-aSyn stain in skin samples from subjects with PD or non-PD subjects (see FIG. 21 and FIG. 22). This finding presented the opportunity for a pS129-aSyn assay with potentially higher specificity for aggregated aSyn. However, short Protease 3 treatment (4 minutes) did not completely eliminate granular pS129-aSyn stain while Protease 2 or extended Protease 3 treatment (>4 minutes) resulted in poor tissue morphology when tissue was subsequently subjected to CC2 at 100° C. for PGP9.5 detection (data not shown).

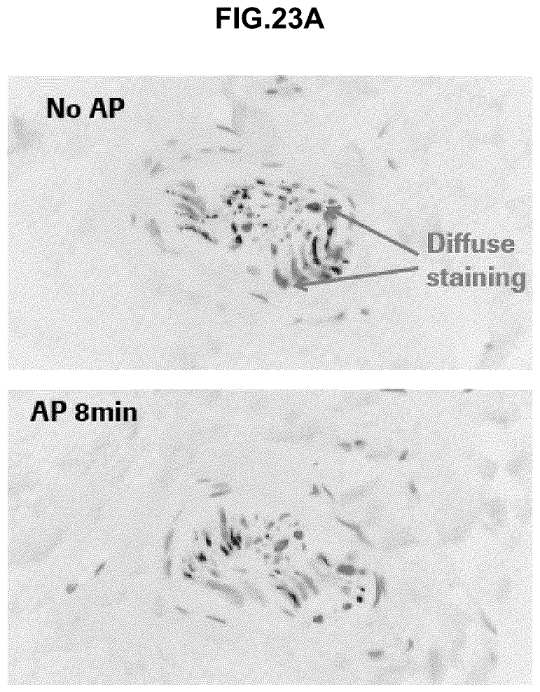

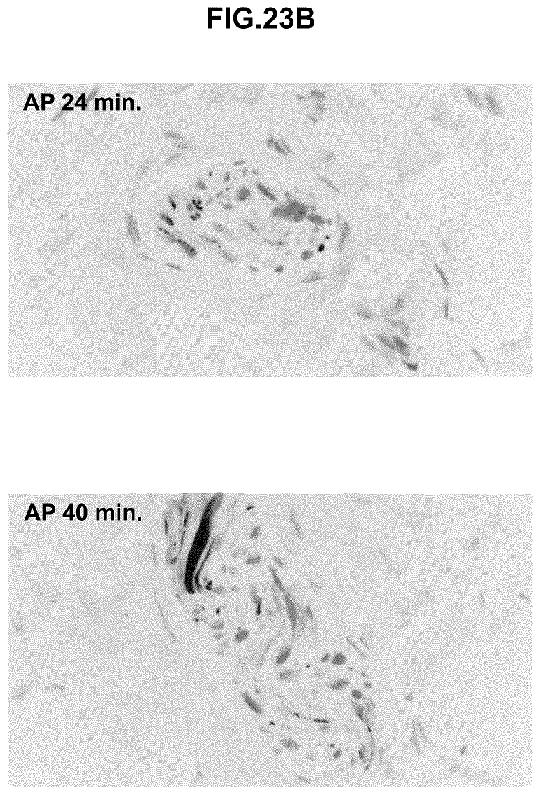

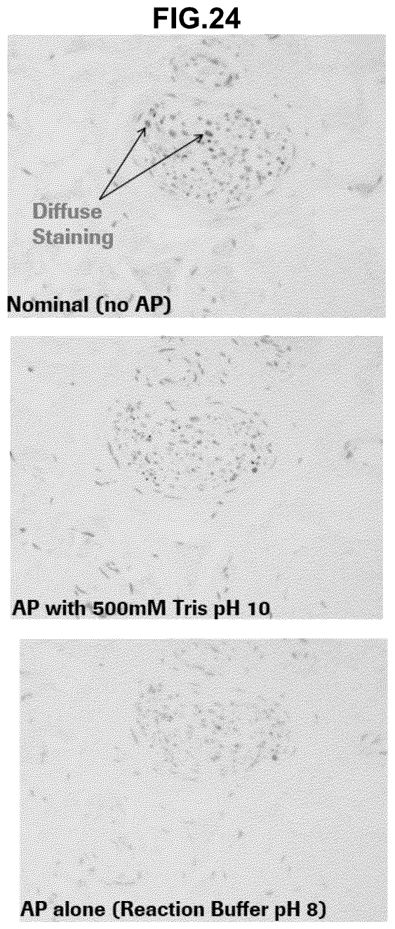

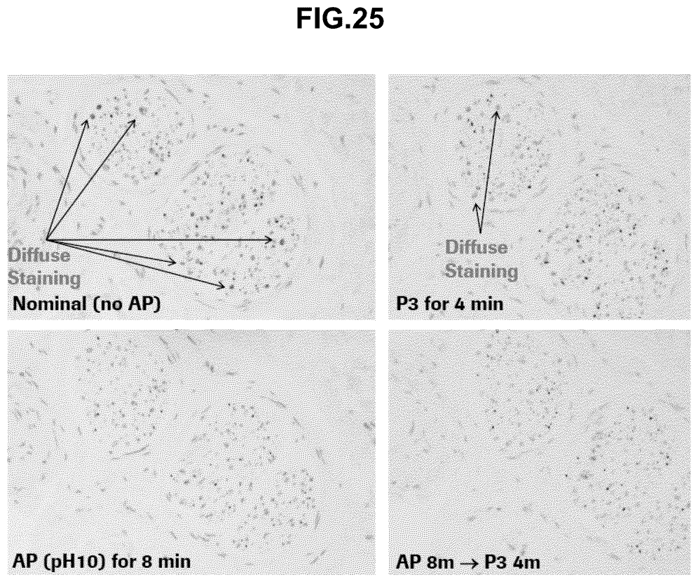

The possibility of a more specific assay protocol that could completely eliminate the granular type of pS129-aSyn stain present in both PD and non-PD samples while preserving the discrete type of pS129-aSyn stain seen only in PD samples led to the search for an alternative method to remove granular pS129-aSyn stain. Once S129 residue becomes phosphorylated in aggregated aSyn, the attached phosphate group is more resistant to removal by protein phosphatases than phospho-S129 in soluble aSyn (Waxman & Giasson, J Neuropathol Exp Neurol 67(5):402-416 (May 2008)). The ability of mammalian alkaline phosphatase, a phosphatase with wide substrate specificity and shown to be active toward serine-phosphorylated protein, was tested to selectively remove granular pS129-aSyn stain in FFPE skin sections. Treatment of deparaffinized skin sections with purified endogenous (New England Biolabs, NEB) or recombinant (Roche) calf intestinal alkaline phosphatase at pH 9 or above prior to 7E2 antibody incubation prevented the appearance of granular pS129-aSyn stain without significantly impacting the level of discrete pS129-aSyn stain (see FIG. 23A and FIG. 23B). Interestingly, alkaline phosphatase treatment at pH 8 did result in decreased staining of discrete pS129-aSyn (see FIG. 24). Sequential treatments first with alkaline phosphatase to remove S129 phosphorylation in soluble aSyn followed by protease-mediated enhancement of antigen retrieval formed the basis of an alternative pS129-aSyn and PGP9.5 silver/yellow dual IHC assay protocol (Bumblebee Assay Protocol 2) with potentially higher specificity toward aggregated aSyn (see FIG. 25).

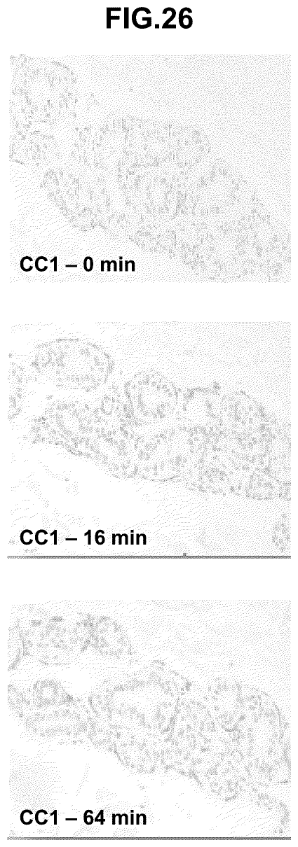

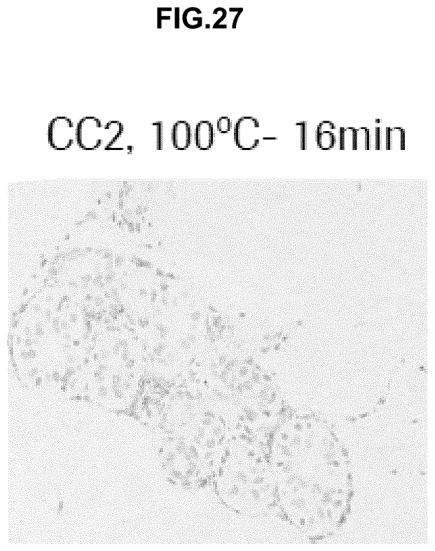

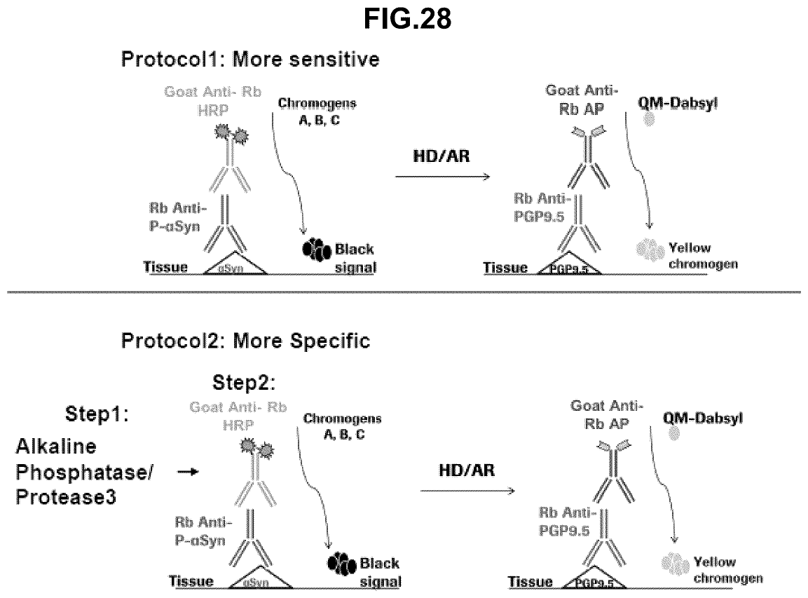

Unlike pS129-aSyn, PGP9.5 detection using EPR4118 antibody was enhanced with increasing durations of antigen retrieval using ULTRA CC1 bulk solution (see FIG. 26). Antigen retrieval with ULTRA CC2 yielded similar enhancement in PGP9.5 staining (see FIG. 27). Automated bright-field duplex IHC assays require sequential antibody incubation and chromogen deposition cycles as shown in FIG. 28. When both primary antibodies of the same species are used in a dual IHC assay protocol, a heat deactivation (HD) step in the form of ULTRA CC2 incubation at 100° C. is needed to remove the primary and secondary antibodies deposited in the first cycle to prevent potential cross-reaction of primary antibody from the first cycle with secondary antibody from the second cycle. Although a heat deactivation step is not necessary when the chromogen deposited in the first cycle is silver/black (yellow stain deposited on top of black stain remains black), treatment with CC2 for 16 minutes at 100° C. serves as antigen retrieval for PGP9.5 staining using EPR4118 antibody.

### Example 6. Assay Reproducibility and Precision

The results of the experiments described in preceding sections led to two optimized protocols for the automated phospho-S129-alpha-synunclein and PGP9.5 silver/yellow dual IHC assay (Bumblebee Assay Protocol 1 and Protocol 2). As detailed in the section above, Protocol 1 contains no antigen retrieval steps for pS129-aSyn detection. Protocol 2 is identical to Protocol 1 in every aspect except the samples were treated with alkaline phosphatase followed by protease after deparaffinization and before 7E2 antibody incubation. The intra- and inter-run reproducibility of Bumblebee Assay Protocols 1 and 2 was assessed in scalp samples from three different subjects with PD. For intra-run reproducibility, 5 consecutively sectioned slides from each PD subject were stained using Protocol 1 or 2 as described in Methods and the numbers of nerve features with and without discrete pS129-aSyn were enumerated by a pathologist. The amount of skin nerve features with discrete pS129-aSyn stain as percentage of total nerve features in each PD subject were averaged from the 5 stained sections and shown in Table 11.

TABLE 11
Reproducibility (intra-run) of Protocol
1 and Protocol 2 in Skin Samples

| | Average % of nerve features | Std. | % |
|---|---|---|---|
| | with discrete p-aSyn staining | Dev. | CV |
| Protocol 1 | | | |
| Sample 1 | 91 | 2 | 2 |
| Sample 2 | 87 | 1 | 2 |
| Sample 3 | 87 | 2 | 3 |
| Protocol 2 | | | |
| Sample 1 | 76 | 4 | 5 |
| Sample 2 | 63 | 11 | 17 |
| Sample 3 | 91 | 2 | 3 |

For inter-run reproducibility, triplicate slides from each of three different subjects with PD were stained using Protocol 1 or 2 on three different instruments over non-consecutive days. Within a run, the three slides from each PD subject were consecutively cut sections; however, sections were not continuous between different runs. The average percentages of nerve features with discrete pS129-aSyn stain from each of the three runs are shown in Table 12.

TABLE 12
Reproducibility (inter-run) of Protocol
1 and Protocol 2 in Skin Samples

| | Average % of nerve features with | | | | | |
|---|---|---|---|---|---|---|
| | Discrete p-aSyn staining | | | Average | Std. | % |
| | Run 1 | Run 2 | Run 3 | of 3 runs | Dev. | CV |
| Protocol 1 | | | | | | |
| Sample 1 | 91 | 87 | 89 | 89 | 3 | 3 |
| Sample 2 | 87 | 88 | 82 | 86 | 3 | 4 |
| Sample 3 | 87 | 75 | 69* | 77 | 9 | 12 |
| Protocol 2 | | | | | | |
| Sample 1 | 76 | 77 | 81 | 78 | 2 | 3 |
| Sample 2 | 63 | 61 | 50 | 58 | 7 | 12 |
| Sample 3 | 91 | 87 | 65* | 81 | 14 | 17 |

*slides used in Run 3 were more than 20 cuts away from those in Run 1 and contained half as many features with nerves.

In cases where CV exceeded 15% the most likely source of variation is the difference in numbers of nerve features between consecutively sectioned slides. A study of the continuity of nerve features across consecutively sectioned slides has concluded that a difference of 10% to 20% in number of nerve features could be attributed to slide-to-slide variation in feature distribution over 5 to 10 4-μm sections (see Table 13). The intensity of PGP9.5 stained with yellow DABSYL chromogen was 2.5 across all slides stained for assay reproducibility studies.

TABLE 13
Continuity of nerve features in skin tissue

| | | | | |
|---|---|---|---|---|
| | | | | % |
| | | | % | Continuous |
| No | Yes | All | Concordant | w/Ref |

Presence/absence concordance of all nerve-containing features in 20 cases

| | | | | | | |
|---|---|---|---|---|---|---|
| Row: Ref | No | 23 | 1 | 24 | | |
| Column: Ref + 1 | Yes | 4 | 130 | 134 | | 97% |
| | All | 27 | 131 | 158 | 97% | |
| Row: Ref | No | 7 | 17 | 24 | | |
| Column: Ref + 5 | Yes | 15 | 119 | 134 | | 89% |
| | All | 22 | 136 | 158 | 79% | |
| Row: Ref | No | 2 | 22 | 24 | | |
| Column: Ref + 10 | Yes | 26 | 108 | 134 | | 81% |
| | All | 28 | 130 | 158 | 70% | |

Presence/absence concordance of transverse nerve bundles in 20 cases

| | | | | | | |
|---|---|---|---|---|---|---|
| Row: Ref | No | 9 | 1 | 10 | | |
| Column: Ref + 1 | Yes | 3 | 68 | 71 | | 96% |
| | All | 12 | 69 | 81 | 95% | |
| Row: Ref | No | 4 | 6 | 10 | | |
| Column : Ref + 5 | Yes | 8 | 63 | 71 | | 89% |
| | All | 12 | 69 | 81 | 83% | |
| Row: Ref | No | 0 | 10 | 10 | | |
| Column: Ref + 10 | Yes | 15 | 56 | 71 | | 79% |
| | All | 15 | 66 | 81 | 69% | |

Presence/absence concordance of sagittal nerve bundles in 20 cases

| | | | | | | |
|---|---|---|---|---|---|---|
| Row: Ref | No | 6 | 0 | 6 | | |
| Column: Ref + 1 | Yes | 0 | 14 | 14 | | 100% |
| | All | 6 | 14 | 20 | 100% | |
| Row: Ref | No | 1 | 5 | 6 | | |
| Column: Ref + 5 | Yes | 3 | 11 | 14 | | 79% |
| | All | 4 | 16 | 20 | 60% | |
| Row: Ref | No | 2 | 4 | 6 | | |
| Column: Ref + 10 | Yes | 4 | 10 | 14 | | 71% |
| | All | 6 | 14 | 20 | 60% | |

Presence/absence concordance of smooth muscle in 20 cases

| | | | | | | |
|---|---|---|---|---|---|---|
| Row: Ref | No | 4 | 0 | 4 | | |
| Column: Ref + 1 | Yes | 1 | 10 | 11 | | 91% |
| | All | 5 | 10 | 15 | 93% | |
| Row: Ref | No | 2 | 2 | 4 | | |
| Column: Ref + 5 | Yes | 0 | 11 | 11 | | 100% |
| | All | 2 | 13 | 15 | 87% | |
| Row: Ref | No | 0 | 4 | 4 | | |
| Column: Ref + 10 | Yes | 1 | 10 | 11 | | 91% |
| | All | 1 | 14 | 15 | 87% | |

Presence/absence concordance of nerves surrounding glands in 20 cases

| | | | | | | |
|---|---|---|---|---|---|---|
| Row: Ref | No | 3 | 0 | 3 | | |
| Column: Ref + 1 | Yes | 0 | 16 | 16 | | 100% |
| | All | 3 | 16 | 19 | 100%% | |
| Row: Ref | No | 0 | 3 | 3 | | |
| Column: Ref + 5 | Yes | 3 | 13 | 16 | | 81% |
| | All | 3 | 16 | 19 | 68% | |
| Row: Ref | No | 0 | 3 | 3 | | |
| Column: Ref + 10 | Yes | 3 | 13 | 16 | | 81% |
| | All | 3 | 16 | 19 | 68% | |

Presence/absence concordance of nerves surrounding vessels in 20 cases

| | | | | | | |
|---|---|---|---|---|---|---|
| Row: Ref | No | 1 | 0 | 1 | | |
| Column: Ref + 1 | Yes | 0 | 16 | 16 | | 100% |
| | All | 1 | 16 | 17 | 100% | |
| Row: Ref | No | 0 | 1 | 1 | | |
| Column: Ref + 5 | Yes | 1 | 15 | 16 | | 94% |
| | All | 1 | 16 | 17 | 88% | |
| Row: Ref | No | 0 | 1 | 1 | | |
| Column: Ref + 10 | Yes | 2 | 14 | 16 | | 88% |
| | All | 2 | 15 | 17 | 82% | |

Experiments were performed to analyze the precision of pS129-aSyn and PGP9.5 silver/yellow dual IHC assay, and scored on percent of features staining in each section (˜25 features). To analyze inter-run precision, three runs of three slides each on the same instrument were tested at least two days apart (Protocol 1 CV: 2.77% discrete, 5.13% diffuse; Protocol 2 CV: 3.9900 discrete, 0.00% diffuse). To analyze inter-instrument precision, two additional runs of three slides each on a new instrument were performed and compared to first run from first instrument (Protocol 1 CV: 3.22% discrete, 11.86% diffuse; Protocol 2 CV: 3.45% discrete, 0.00% diffuse). In summary, the CVs of both inter-run and inter-instrument precision for both Bumblebee Assay Protocol 1 and 2 were below the 15% threshold needed to qualify for transfer.

### Example 7. Assay Validation—Scalp Samples

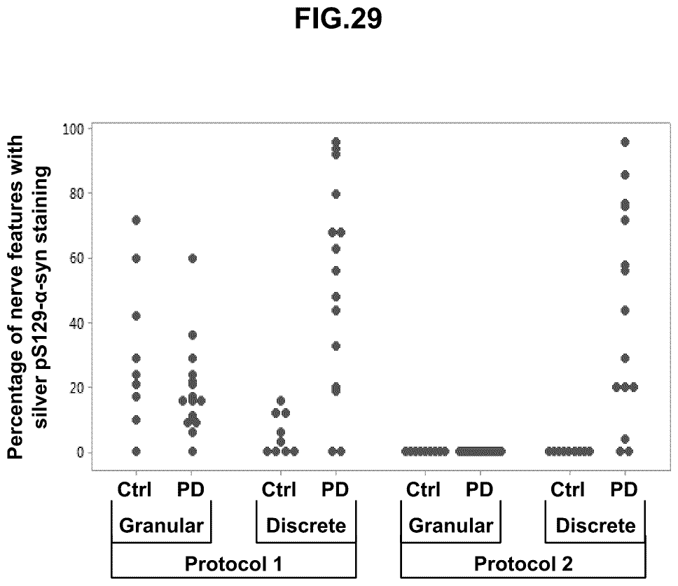

During optimization of phosphorylated-alpha-synuclein and PGP9.5 silver/yellow dual IHC assay (nicknamed Bumblebee Assay) it was observed that smooth discrete phosphorylated-alpha-synuclein staining pattern was exclusively associated with the small cohort of PD scalp samples used for feasibility testing. In order to determine if this trend is extended to a larger cohort of scalp samples from PD and non-PD subjects, scalp sections from 15 PD and 9 non-PD subjects were stained using both Protocol 1 and Protocol 2 of pS129-aSyn and PGP9.5 silver/yellow dual IHC assay (Bumblebee Assay). In addition, sections from a subset of this cohort containing 10 PD and 4 non-PD scalp samples were stained using the automated protease-resistant aSyn DAB IHC assay (pRED/Prothena Assay Protocol). As shown in FIG. 29, 13 of 15 samples (87%) from PD subjects exhibited pS129-aSyn of the discrete type following staining using either Protocol 1 or Protocol 2 of Bumblebee Assay. Of the 9 non-PD scalp samples, 5 (56%) exhibited discrete pS129-aSyn staining using Bumblebee Protocol 1. In contrast, none of the 9 non-PD scalp samples exhibited discrete pS129-aSyn staining using Bumblebee Protocol 2 with mild protease and phosphatase treatments. Diffuse granular type of pS129-aSyn staining was only observed using Protocol 1 but not using Protocol 2.

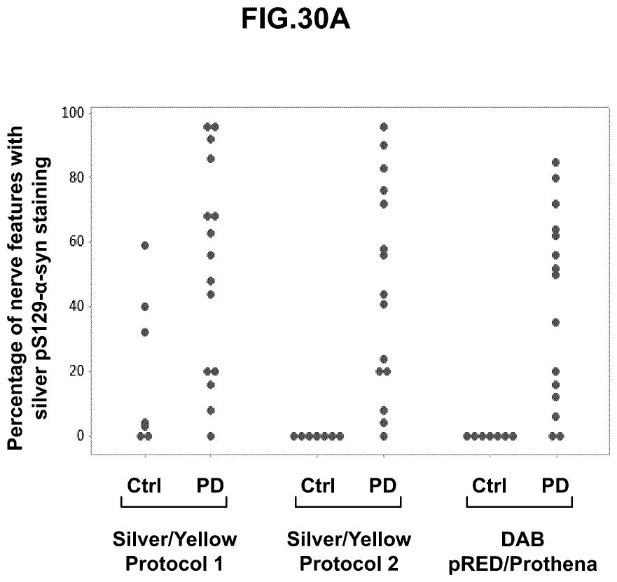

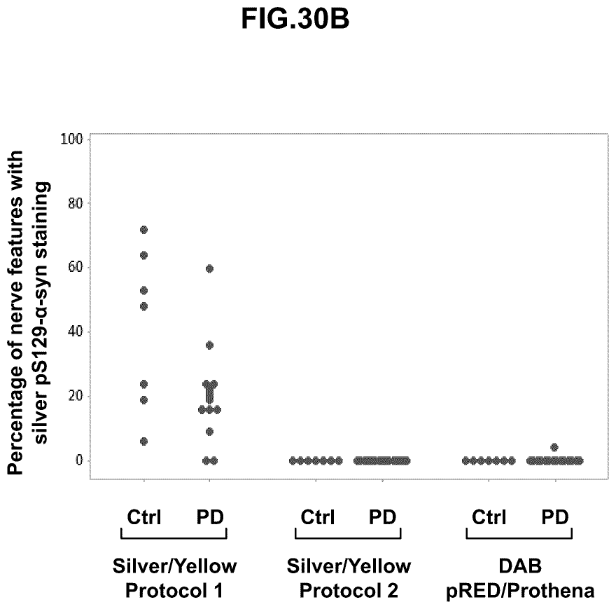

In the subset of subjects in which results from Bumblebee Assay Protocol 1, Bumblebee Assay Protocol 2, and pRED/Prothena Assay Protocol were available, discrete aSyn signals were observed, respectively, in 14, 14, and 13 PD scalp samples out of 15 stained (see FIG. 30A). Of the 4 non-PD scalp samples stained using all three protocols, discrete aSyn stain was not observed with Bumblebee Protocol 2 or pRED/Prothena Protocol (see FIG. 30A). Diffuse granular aSyn stain was observed in both PD and non-PD scalp samples using Bumblebee Protocol 1 (see FIG. 30B). These results suggest that detection of aggregated aSyn using a combination of highly sensitive detection chemistry (metallic silver precipitation) and antibody against pS129-aSyn is likely to have similar sensitivity as an approach based on antibody against modified or un-modified aSyn combined with protease removal of non-aggregated aSyn.

### Example 8. Assay Validation—Abdomen Skin Samples

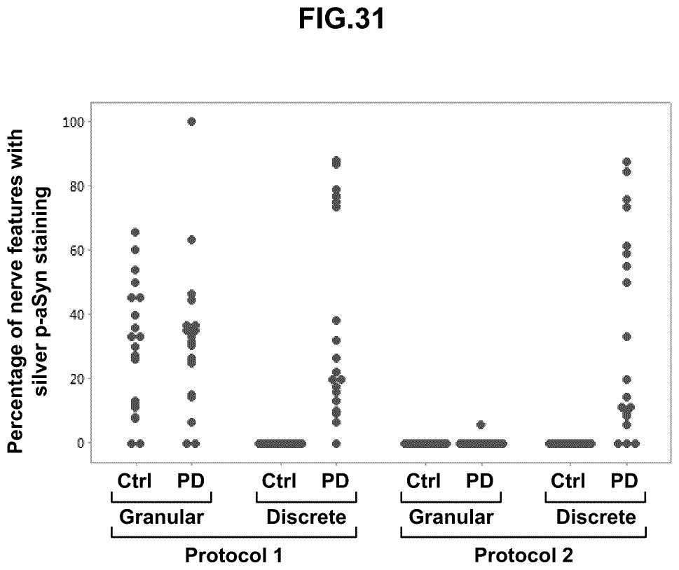

To further assess the sensitivity and specificity of phospho-aSyn and PGP9.5 silver/yellow dual IHC assay, abdomen area skin samples from a cohort of 20 PD and 20 non-PD control subjects were stained using Bumblebee Assay Protocols 1 and 2. The results of the analysis are shown in FIG. 31. Of the 20 PD abdomen skin samples stained using Bumblebee Assay Protocols 1 and 2, discrete pS129-aSyn stain was observed in, respectively, 19 and 17 samples. In contrast, none of the 20 non-PD abdomen skin samples exhibited any discrete pS129-aSyn signal. Also shown in FIG. 31, diffuse granular pS129-aSyn staining was present in most of the PD and non-PD abdomen skin samples stained using Bumblebee Assay Protocol 1 and at a low level in 1 PD sample stained using Bumblebee Assay Protocol 2. These results showed a high degree of association between presence of discrete pS129-aSyn staining and PD clinical status in the expanded cohort of abdomen skin from 20 PD and 20 non-PD subjects (Pearson chi-square=36.2 and 29.6 for Bumblebee Assay Protocols 1 and 2 respectively, P-Values<0.0001 for both protocols).

A cohort of 4 PD and 4 non-PD abdomen skin samples were available in FFPE block format and for testing using both Bumblebee Assay protocols and also the 5C12 antibody- and Protease 1-based pRED/Prothena Assay Protocol. Results of such analysis are shown in the Table 14 below.

TABLE 14
Percentage of nerve features with discrete pS129-aSyn

| | | Percentage of nerve features with discrete pS129-aSyn | | |
|---|---|---|---|---|
| Subject | Subject | Bumblebee Assay | Bumblebee Assay | pRED/Prothena |
| # | Status | Protocol 1 | Protocol 2 | Protocol |
| 1 | Ctrl | 0% | 0% | 0% |
| 2 | Ctrl | 0% | 0% | 3% |
| 3 | Ctrl | 0% | 0% | 0% |
| 4 | Ctrl | 2% | 0% | 0% |
| 5 | PD | 63% | 52% | 22% |
| 6 | PD | 25% | 22% | 24% |
| 7 | PD | 54% | 58% | 33% |
| 8 | PD | 15% | 23% | 3% |

Three of the four PD abdomen skin samples that were tested using the pRED/Prothena Protocol exhibited decreases in percentage of nerve features with discrete pS129-aSyn compared with those tested using either of the Bumblebee Assay protocols. This was not observed in scalp samples stained using all three protocols (FIG. 30). A likely explanation for the discrepancy is that the tissue morphology of the abdomen skin samples was more prone to degradation by Protease 1 treatment than that of the scalp samples. The cause for the apparent differential sensitivity to Protease 1 treatment is unclear at the present. In addition to anatomical site, the abdomen slides had been cut closer to the time they were stained than the scalp slides. In general, it was observed that slides that have been cut and left at room temperature for long period of time (>30 days) before staining are less susceptible to protease-induced morphology degradation (data not shown).

### Example 9. Assay Validation—Pre-S4 Scalp, Colon, and Submandibular Gland Samples

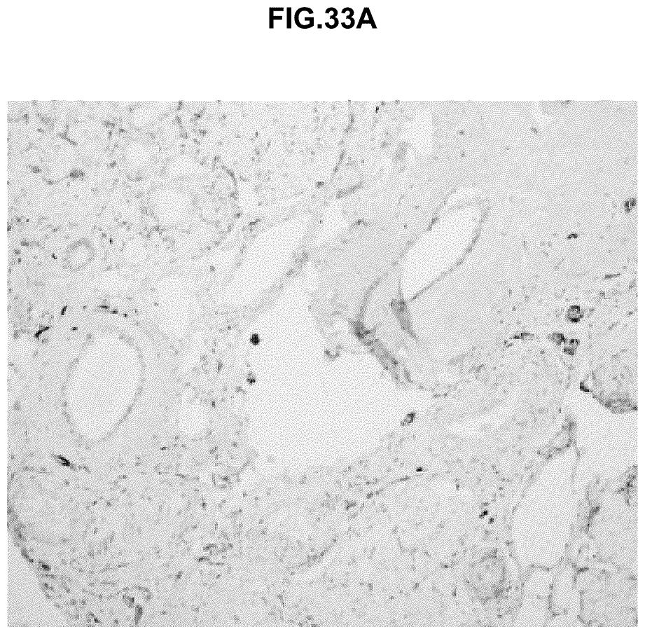

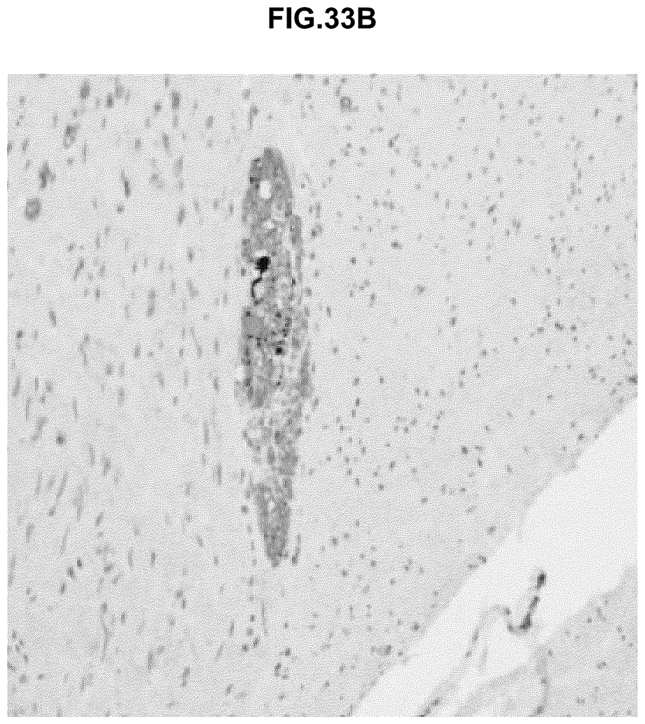

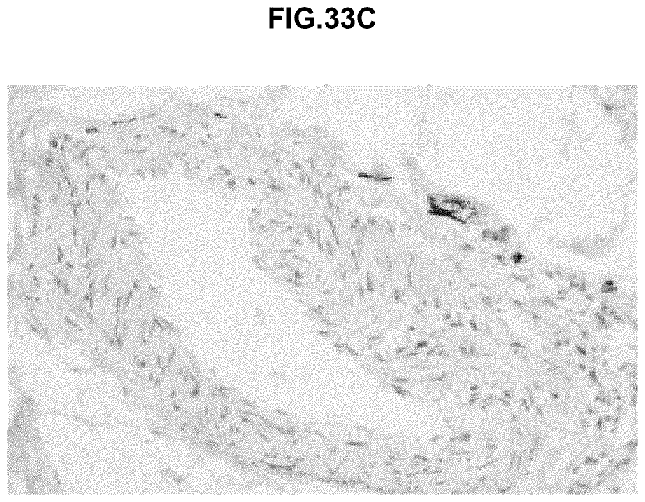

In addition to skin, colon and submandibular gland (SMG) are highly innervated and could be biopsy sites for detection of aggregated alpha-synuclein. Duplicate scalp, colon, and SMG samples from 3 PD and 3 non-PD subjects taken at the time of autopsy were stained using Bumblebee Assay Protocol 2. Protocol 1 was not employed because a previous study of normal colon and SMG samples showed Protocol 1 generated heavy discrete phosphorylated alpha-synuclein stain that could be removed by protease and phosphatase treatment using Protocol 2 (see FIG. 32A and FIG. 32B). Typical images of discrete phosphorylated alpha-synuclein stain in the three different tissue sites are shown in (see FIG. 33A-FIG. 33C). The stained slides were evaluated blindly by three independent readers (data not shown). Even though one of the readers used a different scoring method than the other two, there was total agreement in complete segregation of PD from non-PD samples based on presence of discrete phosphorylated alpha-synuclein stain using Bumblebee Protocol 2. Specificity and sensitivity were both 100%.

### Example 10. Assay Validation—Skin Biopsy Samples

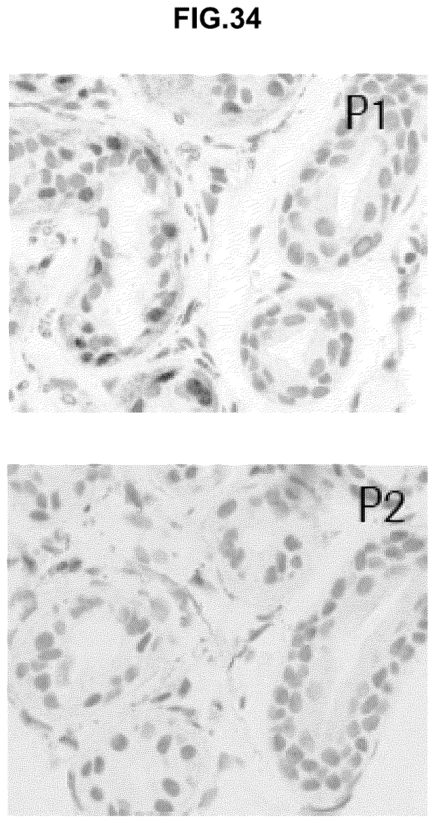

The BSHRI scalp and abdomen skin samples used for assay development and validation were obtained during autopsies. To assess the potential utility of the phosphorylated S129 alpha synuclein and PGP9.5 silver/yellow dual IHC assay for diagnostic applications, FFPE sections of skin punch biopsies obtained during clinical visits from subjects with and without diagnosed PD symptoms were stained using both Bumblebee Assay Protocols 1 and 2. Tissue sections from a total of 72 FFPE blocks from 36 subjects were stained as duplicate blocks from each subject were available. Stained slides were reviewed by pathologists blinded to the clinical status of the subjects. As the samples were small punch biopsies of the skin, numbers of nerve features available per slide were limited (around 5 to 10). As a result, quantitative measurements in the form of percentage of silver-stained nerve features were not made. A slide was considered positive for phosphorylated-aSyn if there were discrete silver stains that were clearly not granular and of sufficient size to distinguish them from background silver dusting. If sections from one of the duplicate blocks were positive for phosphorylated-aSyn stain, the subject was considered positive overall. As in pediatric skin biopsy samples, Bumblebee Protocol 1 generated heavy phosphorylated-aSyn silver stain with discrete morphology that was absent with Protocol 2 (see FIG. 34). This resulted in 93% of the 15 PD and 50% of the 16 control subjects (69% of all 36 subjects including 5 cases of atypical parkinsonism) with positive discrete phosphorylated-aSyn stain using Protocol 1. With Protocol 2, 60% of the 15 PD and 6% of the 16 control subjects (31% of all 36 subjects) exhibited discrete phosphorylated-aSyn signal (see Tables 15 and 16). The unblinded results of each individual tissue block and corresponding PD status for all 36 subjects are shown in Table 16. Chi-square analysis of the results from all 36 subjects demonstrated a highly significant correlation between presence of discrete phosphorylated-aSyn signal and designation of PD status based on clinical symptoms (Pearson Chi-Square=10.506, DF=1, P-Value=0.001). The association remains significant when the 5 subjects with atypical parkinsonism are excluded from analysis (Pearson Chi-Square=10.236, DF=1, P-Value=0.001).

TABLE 15
Phosphu-Ser129 alpha-synuclein (pS129-aSyn) staining in skin
tissue of non-PD subjects using Protocol 1 and Protocol 2

| | Protocol 1 | Protocol 2 |
|---|---|---|
| # (and %) of subjects with either | 25 (69%)* | 11 (31%)* + |
| Block A, or Block B, or both Block | | |
| A and Block B, showing discrete | | |
| pS129-aSyn stain | | |
| # (and %) of subjects with either | 36 (100%)** | 2 (6%)++ |
| Block A, or Block B, or both Block | | |
| A and Block B, showing granular | | |
| pS129-aSyn stain | | |

*Presence of discrete pS129-aSyn using Protocol 1 is defined as having significant amount of discrete stain, particularly around glands, rather than any discrete stain. Definition of “significant” is purely subjective as it is based on qualitative impression of the reader. In contrast, presence of discrete pS129-aSyn using Protocol 2 is defined as having any discrete stain that is specific and not background.
**35 of 36 subjects had granular pS129-aSyn stain in both Block A and Block B using Protocol 1.
+ Of the 11 subjects with discrete pS129-aSyn stain, 5 had discrete stain in both Block A and Block B.
++No subject had granular pS129-aSyn stain in both Block A and Block B using Protocol 2.

TABLE 16
Unblinded phospho-Ser129 alpha-synuclein (pS129-aSyn) staining in
skin tissue of non-PD subjects using Protocol 1 and Protocol 2

| | Presence of Discrete | Presence of Discrete |
|---|---|---|
| | aSyn Staining | aSyn Staining |
| | Protocol 1 | Protocol 2 |
| PD (n = 15) | 14 (93%) | 9 (60%) |
| Control (n = 16) | 8 (50%) | 1 (6%) |

The results demonstrate an automated dual phosphorylated-aSyn and PGP9.5 immunohistochemical assay with high sensitivity and specificity for distinguishing subjects with PD from non-PD control subjects based on presence of the phosphorylated-aSyn stain of the discrete type in skin biopsies. The morphological appearance of discrete phosphorylated-aSyn stain and its resistance to protease and phosphatase treatments are consistent with aggregated aSyn. This assay may have potential value in identifying subjects with PD and/or candidates for therapeutic treatments targeting aggregation of aSyn.

As used herein, the singular forms “a,” “an”, and “the” include plural referents unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

The foregoing is provided for exemplification purposes only and are not intended to limit the scope of the disclosure described in broad terms above. All references cited in this disclosure are incorporated herein by reference.

Although various specific embodiments of the present disclosure have been described herein, it is to be understood that the disclosure is not limited to those precise embodiments and that various changes or modifications can be affected therein by one skilled in the art without departing from the scope and spirit of the disclosure.

The examples given above are merely illustrative and are not meant to be an exhaustive list of all possible embodiments, applications or modifications of the disclosure. Thus, various modifications and variations of the described methods and systems of the disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure which are obvious to those skilled in molecular biology, immunology, chemistry, biochemistry or in the relevant fields are intended to be within the scope of the appended claims.

It is understood that the disclosure is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the disclosure.

The embodiments of the disclosure and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, size, angle size, pressure, time and the like, is, for example, from 1 to 90, specifically from 20 to 80, more specifically from 30 to 70, it is intended that values such as 15 to 85, 22 to 68, 43 to 51, 30 to 32, etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Particular methods, devices, and materials are described, although any methods and materials similar or equivalent to those described herein can be used in the practice or testing of the disclosure. The disclosures of all references and publications cited herein are expressly incorporated by reference in their entireties to the same extent as if each were incorporated by reference individually.

1. A method for determining whether a subject has Parkinson's Disease (PD), the method comprising:
(a) preparing a fixed or frozen section of a biological sample from a subject suspected of having PD;
(b) detecting whether phosphorylated alpha-synuclein localizes within a nerve feature in the section; and
(c) diagnosing the subject with PD when phosphorylated alpha-synuclein localizes within a nerve feature.

2. The method of claim 1, where step (c) further comprises:
contacting the section with a primary antibody capable of binding phosphorylated alpha-synuclein and a primary antibody capable of binding the nerve feature.

3. The method of claim 2, wherein the primary antibody capable of binding phosphorylated alpha-synuclein comprises complementarity determining regions (CDRs) having the following amino acid sequences:
CDR1 VL
(SEQ ID NO: 2)
QSVYNNNN,
CDR2 VL
(SEQ ID NO: 3)
KASKVAS,
CDR3 VL
(SEQ ID NO: 4)
LGGYSGDIYT,
CDR4VH
(SEQ ID NO: 6)
GFTISSYHMS,
CDR5 VH
(SEQ ID NO: 7)
ISTSGNI,
CDR6VH
(SEQ ID NO: 8)
ARLGIATGYSF.

4. The method of claim 2, wherein the primary antibody capable of binding to the nerve feature is selected from an antibody capable of binding a protein selected from the group consisting of: ubiquitin C-terminal hydrolase L1 (PGP9.5, UCHL1, NDGOA; PARK5; PGP95; SPG79; Uch-L1; HEL-117; PGP 9.5; HEL-S-53), RNA binding fox-1 homolog 3 (RBFOX3, FOX3, NEUN, FOX-3, HRNBP3), microtubule associated protein 2 (MAP2, MAP2A, MAP2B, MAP2C), 160 kDa neurofilament medium (NEFM, NFM, NEF3, NF-M), 200 kDa neurofilament heavy (NEFH, NFH, CMT2CC), synaptophysin (SYP, MRX96, MRXSYP), and discs large MAGUK scaffold protein 4 (DLG4, DLGH4, PSD-95, PSD95, SAP-90, SAP90, SAP90A).

5. The method of claim 2, further comprising:
i. contacting the section with a first secondary antibody having a first label conjugated thereto, wherein the first secondary antibody is immunoreactive with the primary antibody capable of binding phosphorylated alpha-synuclein, and
ii. contacting the section with a second secondary antibody having a second label conjugated thereto, wherein the second secondary antibody is immunoreactive with the primary antibody capable of binding the nerve feature.

6. The method of claim 5, further comprising:
i. contacting the section with a set of reagents reactive with the first label of the first secondary antibody to generate a first detectable signal in proximity to phosphorylated alpha-synuclein in the sample;
ii. contacting the section with a set of reagents reactive with the second label of the second secondary antibody to generate a second detectable signal in proximity to the nerve feature in the sample.

7. The method of claim 6, further comprising denaturing immunocomplexes in the sample after contacting the section with the primary antibody capable of binding phosphorylated alpha-synuclein and before contacting the section with the primary antibody capable of binding the nerve feature.

8. The method of claim 1, wherein the section is contacted with at least one protease before being contacted with a primary antibody.

9. The method of claim 8, further comprising contacting the section with at least one phosphatase.

10. The method of claim 2, wherein the primary antibody capable of binding phosphorylated alpha-synuclein and the primary antibody capable of binding the nerve feature are from the same host species.

11. The method of claim 6, wherein the first detectable signal and the second detectable signal are different.

12. The method of claim 11, wherein the first detectable signal is silver stain and the second detectable signal is QM-Dabsyl or Fast-Red.

13. A method of diagnosing PD in a subject, said method comprising:
(a) obtaining a biological sample comprising at least one nerve feature from a subject suspected of having PD;
(b) detecting whether phosphorylated alpha-synuclein localizes with the nerve feature in the sample by contacting the sample with an anti-PGP.5 antibody and determining co-localization between phosphorylated alpha-synuclein and PGP9.5; and
(c) diagnosing the subject with PD when it is determined in the affirmative that there is co-localization between phosphorylated alpha-synuclein and PGP9.5 in the nerve feature.

14. A method of diagnosing and treating PD in a subject, said method comprising:
(a) obtaining a biological sample comprising at least one nerve feature from a subject suspected of having PD;
(b) detecting whether phosphorylated alpha-synuclein localizes with the nerve feature in the sample by contacting the sample with an anti-PGP.5 antibody and determining co-localization between phosphorylated alpha-synuclein and PGP9.5;
(c) diagnosing the subject with PD when it is determined in the affirmative that there is co-localization between phosphorylated alpha-synuclein and PGP9.5 in the nerve feature; and
(d) administering a therapy to treat PD in the subject diagnosed as having PD.

15. A method of treating a subject diagnosed with PD, comprising administering to the subject an effective regime of an alpha-synuclein antibody, wherein an antibody capable of binding phosphorylated alpha-synuclein and an antibody capable of binding a nerve feature have been shown to co-localize in a nerve feature in a skin sample from the subject.

16. The method of claim 15, wherein the effective regime of an alpha-synuclein antibody comprises an alpha-synuclein antibody selected from the group consisting of: a monoclonal antibody binding within residues 1-20 of alpha-synuclein, 1-10 of alpha synuclein, 4-15 of alpha-synuclein, 91-99 of alpha-synuclein, 117-123 of alpha-synuclein, 118-126 of alpha-synuclein, prasinezumab (PRX002), a humanized antibody having the CDR's of antibody clone 1H7 (ATCC Accession No. PTA-8220), a humanized antibody having the CDR's of antibody clone 9E4 (ATCC Accession No. PTA-8221), the CDR's of antibody clone NI-202.21D11, and the CDR's of antibody clone NI-202.12F4, such as, for example, alpha-synuclein antibodies disclosed in U.S. Pat. Nos. 8,092,801, 8,609,820, 8,790,644, 8,940,276, 9,580,493

17. A method of treating a subject determined to have PD, comprising administering to the subject an effective regime of an alpha-synuclein antibody, wherein the subject was determined to have PD by the method of claim 1.

18. The method of claim 17, wherein the effective regime of an alpha-synuclein antibody comprises an alpha-synuclein antibody selected from the group consisting of: a monoclonal antibody binding within residues 1-20 of alpha-synuclein, 1-10 of alpha synuclein, 4-15 of alpha-synuclein, 91-99 of alpha-synuclein, 117-123 of alpha-synuclein, 118-126 of alpha-synuclein, prasinezumab (PRX002), a humanized antibody having the CDR's of antibody clone 1H7 (ATCC Accession No. PTA-8220), a humanized antibody having the CDR's of antibody clone 9E4 (ATCC Accession No. PTA-8221), the CDR's of antibody clone NI-202.21D11, and the CDR's of antibody clone NI-202.12F4, such as, for example, alpha-synuclein antibodies disclosed in U.S. Pat. Nos. 8,092,801, 8,609,820, 8,790,644, 8,940,276, 9,580,493.
